SEQUENCE LISTING (1247 sequences)

SEQ ID NO: 1 - protein, 9 aa, Homo sapiens
ILVSHLSGV

SEQ ID NO: 2 - protein, 10 aa, Homo sapiens
GVDQGTCLHK

SEQ ID NO: 3 - protein, 8 aa, Homo sapiens
NAMQKILV

SEQ ID NO: 4 - protein, 9 aa, Homo sapiens
MQKILVSHL

SEQ ID NO: 5 - protein, 10 aa, Homo sapiens
KILVSHLSGV

SEQ ID NO: 6 - protein, 9 aa, Homo sapiens
ARNLLLWAL

SEQ ID NO: 7 - protein, 9 aa, Homo sapiens
RESPGQKDL

SEQ ID NO: 8 - protein, 10 aa, Homo sapiens
STGMDGTAVW

SEQ ID NO: 9 - protein, 9 aa, Homo sapiens
TGMDGTAVW

SEQ ID NO: 10 - protein, 9 aa, Homo sapiens
AFIIHPWHR

SEQ ID NO: 11 - protein, 10 aa, Homo sapiens
GEGAFIIHPW

SEQ ID NO: 12 - protein, 11 aa, Homo sapiens
RSTGMDGTAVW

SEQ ID NO: 13 - protein, 9 aa, Homo sapiens
QGARNLLLW

SEQ ID NO: 14 - protein, 9 aa, Homo sapiens
QHAHRSTGM

SEQ ID NO: 15 - protein, 10 aa, Homo sapiens
GAFIIHPWHR

SEQ ID NO: 16 - protein, 8 aa, Homo sapiens
FIIHPWHR

SEQ ID NO: 17 - protein, 9 aa, Homo sapiens
WALHQHAHR

SEQ ID NO: 18 - protein, 8 aa, Homo sapiens
HAHRSTGM

SEQ ID NO: 19 - protein, 10 aa, Homo sapiens
KRARESPGQK

SEQ ID NO: 20 - protein, 9 aa, Homo sapiens
RARESPGQK

SEQ ID NO: 21 - protein, 9 aa, Homo sapiens
EGAFIIHPW

SEQ ID NO: 22 - protein, 10 aa, Homo sapiens
AARCLDRGQW

SEQ ID NO: 23 - protein, 8 aa, Homo sapiens
GARNLLLW

SEQ ID NO: 24 - protein, 10 aa, Homo sapiens
ARCLDRGQWL

SEQ ID NO: 25 - protein, 9 aa, Homo sapiens
GEGAFIIHP

SEQ ID NO: 26 - protein, 11 aa, Homo sapiens
EGAFIIHPWHR

SEQ ID NO: 27 - protein, 9 aa, Homo sapiens
KDLQGARNL

SEQ ID NO: 28 - protein, 10 aa, Homo sapiens
GARNLLLWAL

SEQ ID NO: 29 - protein, 9 aa, Homo sapiens
LQGARNLLL

SEQ ID NO: 30 - protein, 9 aa, Homo sapiens
TARENLSRY

SEQ ID NO: 31 - protein, 9 aa, Homo sapiens
RENLSRYQM

SEQ ID NO: 32 - protein, 9 aa, Homo sapiens
KTQDKCFLK

SEQ ID NO: 33 - protein, 9 aa, Homo sapiens
STARENLSR

SEQ ID NO: 34 - protein, 9 aa, Homo sapiens
IFSTARENL

SEQ ID NO: 35 - protein, 10 aa, Homo sapiens
STARENLSRY

SEQ ID NO: 36 - protein, 8 aa, Homo sapiens
SRYQMLHY

SEQ ID NO: 37 - protein, 10 aa, Homo sapiens
NTCGIFSTAR

SEQ ID NO: 38 - protein, 10 aa, Homo sapiens
ARENLSRYQM

SEQ ID NO: 39 - protein, 9 aa, Homo sapiens
LSRYQMLHY

SEQ ID NO: 40 - protein, 9 aa, Homo sapiens
ENLSRYQML

SEQ ID NO: 41 - protein, 8 aa, Homo sapiens
NLSRYQML

SEQ ID NO: 42 - protein, 10 aa, Homo sapiens
FSTARENLSR

SEQ ID NO: 43 - protein, 9 aa, Homo sapiens
SRYQMLHYK

SEQ ID NO: 44 - protein, 10 aa, Homo sapiens
RENLSRYQML

SEQ ID NO: 45 - protein, 10 aa, Homo sapiens
NLSRYQMLHY

SEQ ID NO: 46 - protein, 10 aa, Homo sapiens
GVDQGTCLTK

SEQ ID NO: 47 - protein, 9 aa, Homo sapiens
ILVSHLSEL

SEQ ID NO: 48 - protein, 8 aa, Homo sapiens
NAMQKICL

SEQ ID NO: 49 - protein, 11 aa, Homo sapiens
SELIKEPVSTK

SEQ ID NO: 50 - protein, 10 aa, Homo sapiens
ARESPGQKEI

SEQ ID NO: 51 - protein, 10 aa, Homo sapiens
ARESPGRKIF

SEQ ID NO: 52 - protein, 9 aa, Homo sapiens
ESPGRKIFR

SEQ ID NO: 53 - protein, 9 aa, Homo sapiens
GQKEIFRGL

SEQ ID NO: 54 - protein, 10 aa, Homo sapiens
GRKIFRGLEI

SEQ ID NO: 55 - protein, 9 aa, Homo sapiens
KEIFRGLEI

SEQ ID NO: 56 - protein, 9 aa, Homo sapiens
RARESPGRK

SEQ ID NO: 57 - protein, 9 aa, Homo sapiens
RESPGQKEI

SEQ ID NO: 58 - protein, 10 aa, Homo sapiens
RESPGQKEIF

SEQ ID NO: 59 - protein, 8 aa, Homo sapiens
RESPGRKI

SEQ ID NO: 60 - protein, 9 aa, Homo sapiens
RESPGRKIF

SEQ ID NO: 61 - protein, 10 aa, Homo sapiens
SPGRKIFRGL

SEQ ID NO: 62 - protein, 9 aa, Homo sapiens
GIFSTAREK

SEQ ID NO: 63 - protein, 10 aa, Homo sapiens
AREKVSDASL

SEQ ID NO: 64 - protein, 10 aa, Homo sapiens
ARENLSDASL

SEQ ID NO: 65 - protein, 9 aa, Homo sapiens
ENLSRFSEI

SEQ ID NO: 66 - protein, 10 aa, Homo sapiens
GIFSTAREKK

SEQ ID NO: 67 - protein, 9 aa, Homo sapiens
IFSTARQVF

SEQ ID NO: 68 - protein, 9 aa, Homo sapiens
IFSTASVQV

SEQ ID NO: 69 - protein, 9 aa, Homo sapiens
KEDSTKQVF

SEQ ID NO: 70 - protein, 9 aa, Homo sapiens
KTQDISSEI

SEQ ID NO: 71 - protein, 9 aa, Homo sapiens
MLHYKTQDI

SEQ ID NO: 72 - protein, 9 aa, Homo sapiens
MLHYKTQEI

SEQ ID NO: 73 - protein, 9 aa, Homo sapiens
NTCGIFSTI

SEQ ID NO: 74 - protein, 10 aa, Homo sapiens
QEIEDSTKQV

SEQ ID NO: 75 - protein, 11 aa, Homo sapiens
QEIEDSTKQVF

SEQ ID NO: 76 - protein, 9 aa, Homo sapiens
REKKSVQVS

SEQ ID NO: 77 - protein, 9 aa, Homo sapiens
REKVSDASL

SEQ ID NO: 78 - protein, 9 aa, Homo sapiens
RENLSDASL

SEQ ID NO: 79 - protein, 9 aa, Homo sapiens
RENTAIRTP

SEQ ID NO: 80 - protein, 11 aa, Homo sapiens
SRFSEIEDSTK

SEQ ID NO: 81 - protein, 10 aa, Homo sapiens
STARENLSRF

SEQ ID NO: 82 - protein, 9 aa, Homo sapiens
STARENTAI

SEQ ID NO: 83 - protein, 10 aa, Homo sapiens
STARENTAIR

SEQ ID NO: 84 - protein, 8 aa, Homo sapiens
TAREKKSV

SEQ ID NO: 85 - protein, 11 aa, Homo sapiens
TAREKVSDASL

SEQ ID NO: 86 - protein, 9 aa, Homo sapiens
TARENLSRF

SEQ ID NO: 87 - protein, 8 aa, Homo sapiens
TARENTAI

SEQ ID NO: 88 - protein, 9 aa, Homo sapiens
TARENTAIR

SEQ ID NO: 89 - protein, 9 aa, Homo sapiens
TARQVFSEI

SEQ ID NO: 90 - protein, 9 aa, Homo sapiens
AAENQMTIL

SEQ ID NO: 91 - protein, 10 aa, Homo sapiens
AAENQMTILK

SEQ ID NO: 92 - protein, 9 aa, Homo sapiens
AAVPISCKK

SEQ ID NO: 93 - protein, 9 aa, Homo sapiens
AAVPISCKR

SEQ ID NO: 94 - protein, 11 aa, Homo sapiens
AEESLHKNNNY

SEQ ID NO: 95 - protein, 10 aa, Homo sapiens
AEKHKGEEHW

SEQ ID NO: 96 - protein, 8 aa, Homo sapiens
AENQMTIL

SEQ ID NO: 97 - protein, 9 aa, Homo sapiens
AENQMTILK

SEQ ID NO: 98 - protein, 10 aa, Homo sapiens
AENQMTILKT

SEQ ID NO: 99 - protein, 9 aa, Homo sapiens
AETYNSWKV

SEQ ID NO: 100 - protein, 10 aa, Homo sapiens
AETYNSWKVK

SEQ ID NO: 101 - protein, 8 aa, Homo sapiens
AEWNKQKL

SEQ ID NO: 102 - protein, 9 aa, Homo sapiens
AEWNKQKLP

SEQ ID NO: 103 - protein, 8 aa, Homo sapiens
AEYEPNLF

SEQ ID NO: 104 - protein, 9 aa, Homo sapiens
AEYEPNLFK

SEQ ID NO: 105 - protein, 10 aa, Homo sapiens
AEYEPNLFKT

SEQ ID NO: 106 - protein, 9 aa, Homo sapiens
AFRIASGNK

SEQ ID NO: 107 - protein, 10 aa, Homo sapiens
AFRIASGNKK

SEQ ID NO: 108 - protein, 9 aa, Homo sapiens
AHGTKLNVL

SEQ ID NO: 109 - protein, 10 aa, Homo sapiens
AIKLSISNSY

SEQ ID NO: 110 - protein, 10 aa, Homo sapiens
ALGFSWTPSR

SEQ ID NO: 111 - protein, 9 aa, Homo sapiens
ALLLKKLLK

SEQ ID NO: 112 - protein, 9 aa, Homo sapiens
ALSIHWDMK

SEQ ID NO: 113 - protein, 9 aa, Homo sapiens
APGALCPAL

SEQ ID NO: 114 - protein, 9 aa, Homo sapiens
APKKNRLTT

SEQ ID NO: 115 - protein, 9 aa, Homo sapiens
APLLPSVCL

SEQ ID NO: 116 - protein, 11 aa, Homo sapiens
APPYNSEPAEY

SEQ ID NO: 117 - protein, 8 aa, Homo sapiens
AQSPRKLY

SEQ ID NO: 118 - protein, 9 aa, Homo sapiens
AQSPRKLYR

SEQ ID NO: 119 - protein, 9 aa, Homo sapiens
ASVPVKTQI

SEQ ID NO: 120 - protein, 10 aa, Homo sapiens
ASVPVKTQIK

SEQ ID NO: 121 - protein, 9 aa, Homo sapiens
ASVTDSENK

SEQ ID NO: 122 - protein, 10 aa, Homo sapiens
ASVTVKTNQR

SEQ ID NO: 123 - protein, 9 aa, Homo sapiens
ASVTVKTQI

SEQ ID NO: 124 - protein, 9 aa, Homo sapiens
ASVTVKTQR

SEQ ID NO: 125 - protein, 9 aa, Homo sapiens
ATTVPLQTI

SEQ ID NO: 126 - protein, 9 aa, Homo sapiens
ATYHLQPLH

SEQ ID NO: 127 - protein, 9 aa, Homo sapiens
ATYHLTSAL

SEQ ID NO: 128 - protein, 10 aa, Homo sapiens
ATYHLTSALK

SEQ ID NO: 129 - protein, 9 aa, Homo sapiens
CKKTSGNSF

SEQ ID NO: 130 - protein, 9 aa, Homo sapiens
CKYKHPGSF

SEQ ID NO: 131 - protein, 8 aa, Homo sapiens
CPALGFSW

SEQ ID NO: 132 - protein, 8 aa, Homo sapiens
CPYLIVII

SEQ ID NO: 133 - protein, 9 aa, Homo sapiens
CPYLIVIIL

SEQ ID NO: 134 - protein, 9 aa, Homo sapiens
CQSGTAETY

SEQ ID NO: 135 - protein, 8 aa, Homo sapiens
DALSICRI

SEQ ID NO: 136 - protein, 9 aa, Homo sapiens
DANAYPQTK

SEQ ID NO: 137 - protein, 9 aa, Homo sapiens
DECSTHKVF

SEQ ID NO: 138 - protein, 10 aa, Homo sapiens
DECSTHSQGF

SEQ ID NO: 139 - protein, 8 aa, Homo sapiens
DEKDQRTY

SEQ ID NO: 140 - protein, 11 aa, Homo sapiens
DENEVGFRGSW

SEQ ID NO: 141 - protein, 10 aa, Homo sapiens
DESLKKNEIY

SEQ ID NO: 142 - protein, 9 aa, Homo sapiens
DESLKKNRF

SEQ ID NO: 143 - protein, 9 aa, Homo sapiens
DESLTVKTQ

SEQ ID NO: 144 - protein, 9 aa, Homo sapiens
DFNSNHNIL

SEQ ID NO: 145 - protein, 9 aa, Homo sapiens
DFYKTHIVY

SEQ ID NO: 146 - protein, 9 aa, Homo sapiens
DGENTPIAY

SEQ ID NO: 147 - protein, 10 aa, Homo sapiens
DGENTPIAYF

SEQ ID NO: 148 - protein, 9 aa, Homo sapiens
DGKIPLLHI

SEQ ID NO: 149 - protein, 9 aa, Homo sapiens
DIFTDNENK

SEQ ID NO: 150 - protein, 9 aa, Homo sapiens
DIFTDSFSY

SEQ ID NO: 151 - protein, 10 aa, Homo sapiens
DILHNSLEDY

SEQ ID NO: 152 - protein, 9 aa, Homo sapiens
DIVTESVPV

SEQ ID NO: 153 - protein, 9 aa, Homo sapiens
DKISKEVVL

SEQ ID NO: 154 - protein, 10 aa, Homo sapiens
DLNADPLCKR

SEQ ID NO: 155 - protein, 9 aa, Homo sapiens
DLNADPLCW

SEQ ID NO: 156 - protein, 9 aa, Homo sapiens
DLNADPLCY

SEQ ID NO: 157 - protein, 10 aa, Homo sapiens
DLNADPLCYR

SEQ ID NO: 158 - protein, 9 aa, Homo sapiens
DLNFWKLRK

SEQ ID NO: 159 - protein, 9 aa, Homo sapiens
DLSDLNFWK

SEQ ID NO: 160 - protein, 10 aa, Homo sapiens
DLSDLNFWKY

SEQ ID NO: 161 - protein, 9 aa, Homo sapiens
DMKLTTVGK

SEQ ID NO: 162 - protein, 9 aa, Homo sapiens
DMRFISLTK

SEQ ID NO: 163 - protein, 9 aa, Homo sapiens
DPLCWRKEW

SEQ ID NO: 164 - protein, 9 aa, Homo sapiens
DPLCYRKEW

SEQ ID NO: 165 - protein, 9 aa, Homo sapiens
DPLDSPLRV

SEQ ID NO: 166 - protein, 9 aa, Homo sapiens
DPSLQETSL

SEQ ID NO: 167 - protein, 9 aa, Homo sapiens
DQRTYSIGF

SEQ ID NO: 168 - protein, 11 aa, Homo sapiens
DQRTYSIGFSY

SEQ ID NO: 169 - protein, 9 aa, Homo sapiens
DRDKLKPAL

SEQ ID NO: 170 - protein, 10 aa, Homo sapiens
DRFIAASHGF

SEQ ID NO: 171 - protein, 9 aa, Homo sapiens
DRFIASVKT

SEQ ID NO: 172 - protein, 9 aa, Homo sapiens
DRFIASVPV

SEQ ID NO: 173 - protein, 9 aa, Homo sapiens
DRFIASVTE

SEQ ID NO: 174 - protein, 10 aa, Homo sapiens
DRFIASVTEA

SEQ ID NO: 175 - protein, 11 aa, Homo sapiens
DRFIASVTEAA

SEQ ID NO: 176 - protein, 9 aa, Homo sapiens
DRFIASVTV

SEQ ID NO: 177 - protein, 9 aa, Homo sapiens
DRFTNQREA

SEQ ID NO: 178 - protein, 10 aa, Homo sapiens
DRFTNQREAA

SEQ ID NO: 179 - protein, 9 aa, Homo sapiens
DRLWPVNGL

SEQ ID NO: 180 - protein, 10 aa, Homo sapiens
DRLWPVNGLL

SEQ ID NO: 181 - protein, 9 aa, Homo sapiens
DRQTVKTQI

SEQ ID NO: 182 - protein, 10 aa, Homo sapiens
DSENTASHGF

SEQ ID NO: 183 - protein, 10 aa, Homo sapiens
DSENTNQRGF

SEQ ID NO: 184 - protein, 9 aa, Homo sapiens
DSKNKINDM

SEQ ID NO: 185 - protein, 9 aa, Homo sapiens
DSNVAQSPR

SEQ ID NO: 186 - protein, 9 aa, Homo sapiens
DSNVGSPLR

SEQ ID NO: 187 - protein, 9 aa, Homo sapiens
DSQQVSRRW

SEQ ID NO: 188 - protein, 9 aa, Homo sapiens
DSSALEDLW

SEQ ID NO: 189 - protein, 9 aa, Homo sapiens
DTDPLDSPL

SEQ ID NO: 190 - protein, 9 aa, Homo sapiens
DVARIHKVF

SEQ ID NO: 191 - protein, 8 aa, Homo sapiens
DVLSNLVI

SEQ ID NO: 192 - protein, 9 aa, Homo sapiens
DVLSNLVMK

SEQ ID NO: 193 - protein, 10 aa, Homo sapiens
DVLSNLVMKR

SEQ ID NO: 194 - protein, 8 aa, Homo sapiens
DVPWITLI

SEQ ID NO: 195 - protein, 9 aa, Homo sapiens
EALDDSEDM

SEQ ID NO: 196 - protein, 9 aa, Homo sapiens
EALGFVYHL

SEQ ID NO: 197 - protein, 9 aa, Homo sapiens
EDILHNSVF

SEQ ID NO: 198 - protein, 9 aa, Homo sapiens
EDLSDLNFW

SEQ ID NO: 199 - protein, 11 aa, Homo sapiens
EDLSDLNFWKY

SEQ ID NO: 200 - protein, 9 aa, Homo sapiens
EDVPWITLI

SEQ ID NO: 201 - protein, 10 aa, Homo sapiens
EEGGKALEDF

SEQ ID NO: 202 - protein, 10 aa, Homo sapiens
EEHLKALEDF

SEQ ID NO: 203 - protein, 9 aa, Homo sapiens
EEHSMSPEM

SEQ ID NO: 204 - protein, 10 aa, Homo sapiens
EEHWKALEDF

SEQ ID NO: 205 - protein, 9 aa, Homo sapiens
EELVTLHPA

SEQ ID NO: 206 - protein, 9 aa, Homo sapiens
EEQSTVSTI

SEQ ID NO: 207 - protein, 10 aa, Homo sapiens
EESELDALSI

SEQ ID NO: 208 - protein, 10 aa, Homo sapiens
EESLHKNNNY

SEQ ID NO: 209 - protein, 9 aa, Homo sapiens
EETDITESV

SEQ ID NO: 210 - protein, 9 aa, Homo sapiens
EETDIVTES

SEQ ID NO: 211 - protein, 10 aa, Homo sapiens
EETDIVTESV

SEQ ID NO: 212 - protein, 9 aa, Homo sapiens
EFIISNQVI

SEQ ID NO: 213 - protein, 9 aa, Homo sapiens
EHLKALEDF

SEQ ID NO: 214 - protein, 11 aa, Homo sapiens
EHLKALEDFTL

SEQ ID NO: 215 - protein, 9 aa, Homo sapiens
EHWKALEDF

SEQ ID NO: 216 - protein, 11 aa, Homo sapiens
EHWKALEDFTL

SEQ ID NO: 217 - protein, 9 aa, Homo sapiens
EIKKKSTTK

SEQ ID NO: 218 - protein, 9 aa, Homo sapiens
EKDQRTYSI

SEQ ID NO: 219 - protein, 9 aa, Homo sapiens
ELVTLHPAK

SEQ ID NO: 220 - protein, 10 aa, Homo sapiens
ENEVGFRGSW

SEQ ID NO: 221 - protein, 9 aa, Homo sapiens
ENFEEQSTV

SEQ ID NO: 222 - protein, 9 aa, Homo sapiens
ENNHSISTR

SEQ ID NO: 223 - protein, 10 aa, Homo sapiens
ENSLPRISSR

SEQ ID NO: 224 - protein, 10 aa, Homo sapiens
ENTASHGFGK

SEQ ID NO: 225 - protein, 9 aa, Homo sapiens
ENTPIAYFF

SEQ ID NO: 226 - protein, 9 aa, Homo sapiens
EPAEYEPNL

SEQ ID NO: 227 - protein, 10 aa, Homo sapiens
EPAEYEPNLF

SEQ ID NO: 228 - protein, 9 aa, Homo sapiens
EPNTVNITA

SEQ ID NO: 229 - protein, 11 aa, Homo sapiens
EPNTVNITAGF

SEQ ID NO: 230 - protein, 9 aa, Homo sapiens
EPQSQLVLA

SEQ ID NO: 231 - protein, 11 aa, Homo sapiens
EPQSQLVLATY

SEQ ID NO: 232 - protein, 9 aa, Homo sapiens
EPRRVTSQM

SEQ ID NO: 233 - protein, 10 aa, Homo sapiens
EQEHQAAGAW

SEQ ID NO: 234 - protein, 9 aa, Homo sapiens
EQKAFNHSR

SEQ ID NO: 235 - protein, 9 aa, Homo sapiens
EQKALSIHW

SEQ ID NO: 236 - protein, 10 aa, Homo sapiens
ERDEKDQRTY

SEQ ID NO: 237 - protein, 9 aa, Homo sapiens
ERIKPVQTV

SEQ ID NO: 238 - protein, 9 aa, Homo sapiens
ERMSLISSL

SEQ ID NO: 239 - protein, 10 aa, Homo sapiens
ERMSLISSLY

SEQ ID NO: 240 - protein, 9 aa, Homo sapiens
ERVLQTEDL

SEQ ID NO: 241 - protein, 9 aa, Homo sapiens
ESKSIFLKV

SEQ ID NO: 242 - protein, 9 aa, Homo sapiens
ESLHKNNNY

SEQ ID NO: 243 - protein, 9 aa, Homo sapiens
ESLKKNEIY

SEQ ID NO: 244 - protein, 10 aa, Homo sapiens
ESLKKNEIYR

SEQ ID NO: 245 - protein, 10 aa, Homo sapiens
ESLKTVKTQR

SEQ ID NO: 246 - protein, 10 aa, Homo sapiens
ESYKLKGNNY

SEQ ID NO: 247 - protein, 10 aa, Homo sapiens
ETDITESVPV

SEQ ID NO: 248 - protein, 9 aa, Homo sapiens
ETDIVTESV

SEQ ID NO: 249 - protein, 9 aa, Homo sapiens
ETFNASFQV

SEQ ID NO: 250 - protein, 10 aa, Homo sapiens
ETFNASFQVI

SEQ ID NO: 251 - protein, 9 aa, Homo sapiens
ETNEQTSKR

SEQ ID NO: 252 - protein, 9 aa, Homo sapiens
ETYNSWKVK

SEQ ID NO: 253 - protein, 10 aa, Homo sapiens
EVGFRGSWHK

SEQ ID NO: 254 - protein, 10 aa, Homo sapiens
EVTKSPRKLY

SEQ ID NO: 255 - protein, 9 aa, Homo sapiens
EVVLTLSGL

SEQ ID NO: 256 - protein, 10 aa, Homo sapiens
EVVPSLACEL

SEQ ID NO: 257 - protein, 9 aa, Homo sapiens
EVVPVNGLL

SEQ ID NO: 258 - protein, 10 aa, Homo sapiens
EWSLRTNPFR

SEQ ID NO: 259 - protein, 9 aa, Homo sapiens
FEDILHNSL

SEQ ID NO: 260 - protein, 9 aa, Homo sapiens
FEEQSTVST

SEQ ID NO: 261 - protein, 10 aa, Homo sapiens
FEEQSTVSTI

SEQ ID NO: 262 - protein, 9 aa, Homo sapiens
FEFTQFRKL

SEQ ID NO: 263 - protein, 9 aa, Homo sapiens
FEKKSPRKL

SEQ ID NO: 264 - protein, 10 aa, Homo sapiens
FEKKSPRKLY

SEQ ID NO: 265 - protein, 9 aa, Homo sapiens
FESGSDRLW

SEQ ID NO: 266 - protein, 8 aa, Homo sapiens
FETEAVAF

SEQ ID NO: 267 - protein, 9 aa, Homo sapiens
FETEAVAFM

SEQ ID NO: 268 - protein, 9 aa, Homo sapiens
FEVGPPVQV

SEQ ID NO: 269 - protein, 8 aa, Homo sapiens
FFPSSHLL

SEQ ID NO: 270 - protein, 10 aa, Homo sapiens
FFPSSHLLKL

SEQ ID NO: 271 - protein, 9 aa, Homo sapiens
FFQTNEASV

SEQ ID NO: 272 - protein, 10 aa, Homo sapiens
FIAASHGFGK

SEQ ID NO: 273 - protein, 9 aa, Homo sapiens
FIASVKTQI

SEQ ID NO: 274 - protein, 10 aa, Homo sapiens
FIASVKTQIK

SEQ ID NO: 275 - protein, 9 aa, Homo sapiens
FIASVTEAA

SEQ ID NO: 276 - protein, 9 aa, Homo sapiens
FIASVTVKH

SEQ ID NO: 277 - protein, 9 aa, Homo sapiens
FIASVTVKT

SEQ ID NO: 278 - protein, 9 aa, Homo sapiens
FIISNQVIL

SEQ ID NO: 279 - protein, 9 aa, Homo sapiens
FKNNSNQAV

SEQ ID NO: 280 - protein, 8 aa, Homo sapiens
FLQRRTKI

SEQ ID NO: 281 - protein, 9 aa, Homo sapiens
FLQRRTKIV

SEQ ID NO: 282 - protein, 10 aa, Homo sapiens
FLRRPNFTRR

SEQ ID NO: 283 - protein, 9 aa, Homo sapiens
FNHSRETSI

SEQ ID NO: 284 - protein, 10 aa, Homo sapiens
FPIKSFVKTL

SEQ ID NO: 285 - protein, 9 aa, Homo sapiens
FPSSHLLKL

SEQ ID NO: 286 - protein, 9 aa, Homo sapiens
FQHLLFGIY

SEQ ID NO: 287 - protein, 10 aa, Homo sapiens
FQISAHSGSL

SEQ ID NO: 288 - protein, 9 aa, Homo sapiens
FQSASHLLR

SEQ ID NO: 289 - protein, 8 aa, Homo sapiens
FQVSKSLY

SEQ ID NO: 290 - protein, 8 aa, Homo sapiens
FRGFYSAL

SEQ ID NO: 291 - protein, 9 aa, Homo sapiens
FRIAGCYEA

SEQ ID NO: 292 - protein, 10 aa, Homo sapiens
FRIAGCYEAL

SEQ ID NO: 293 - protein, 9 aa, Homo sapiens
FRIASGNKK

SEQ ID NO: 294 - protein, 11 aa, Homo sapiens
FRKLMSGFPEV

SEQ ID NO: 295 - protein, 8 aa, Homo sapiens
FRLNGAQM

SEQ ID NO: 296 - protein, 10 aa, Homo sapiens
FRLNGAQMEK

SEQ ID NO: 297 - protein, 8 aa, Homo sapiens
FRSKGAQM

SEQ ID NO: 298 - protein, 8 aa, Homo sapiens
FRSKWAQM

SEQ ID NO: 299 - protein, 8 aa, Homo sapiens
FRSKWSQM

SEQ ID NO: 300 - protein, 8 aa, Homo sapiens
FSDDLNLI

SEQ ID NO: 301 - protein, 9 aa, Homo sapiens
FSDDLNLIL

SEQ ID NO: 302 - protein, 10 aa, Homo sapiens
FSDDLNLILL

SEQ ID NO: 303 - protein, 8 aa, Homo sapiens
FSKVRQVF

SEQ ID NO: 304 - protein, 10 aa, Homo sapiens
FTDNENKSKI

SEQ ID NO: 305 - protein, 9 aa, Homo sapiens
FTDSFSYEA

SEQ ID NO: 306 - protein, 10 aa, Homo sapiens
FTDSFSYEAL

SEQ ID NO: 307 - protein, 9 aa, Homo sapiens
FTRRLQFLY

SEQ ID NO: 308 - protein, 9 aa, Homo sapiens
FVYHLSSEA

SEQ ID NO: 309 - protein, 10 aa, Homo sapiens
FYSAHGTEAL

SEQ ID NO: 310 - protein, 11 aa, Homo sapiens
FYSAHGTKQCF

SEQ ID NO: 311 - protein, 9 aa, Homo sapiens
FYSALQKAV

SEQ ID NO: 312 - protein, 9 aa, Homo sapiens
GECSNQVIL

SEQ ID NO: 313 - protein, 9 aa, Homo sapiens
GEFIISNQV

SEQ ID NO: 314 - protein, 10 aa, Homo sapiens
GEFIISNQVI

SEQ ID NO: 315 - protein, 11 aa, Homo sapiens
GEFIISNQVIL

SEQ ID NO: 316 - protein, 9 aa, Homo sapiens
GENISEKDL

SEQ ID NO: 317 - protein, 10 aa, Homo sapiens
GENISEKDLL

SEQ ID NO: 318 - protein, 9 aa, Homo sapiens
GENTPIAEK

SEQ ID NO: 319 - protein, 11 aa, Homo sapiens
GENTPIAEKDL

SEQ ID NO: 320 - protein, 8 aa, Homo sapiens
GENTPIAY

SEQ ID NO: 321 - protein, 9 aa, Homo sapiens
GENTPIAYF

SEQ ID NO: 322 - protein, 10 aa, Homo sapiens
GENTPIAYFF

SEQ ID NO: 323 - protein, 9 aa, Homo sapiens
GENTPIAYS

SEQ ID NO: 324 - protein, 9 aa, Homo sapiens
GFRGFYSAL

SEQ ID NO: 325 - protein, 9 aa, Homo sapiens
GIAPLLPSV

SEQ ID NO: 326 - protein, 11 aa, Homo sapiens
GISLFSDDLNL

SEQ ID NO: 327 - protein, 9 aa, Homo sapiens
GKALEDFTL

SEQ ID NO: 328 - protein, 10 aa, Homo sapiens
GKESYKGNNY

SEQ ID NO: 329 - protein, 10 aa, Homo sapiens
GLLELTLSGL

SEQ ID NO: 330 - protein, 9 aa, Homo sapiens
GNYLYETTI

SEQ ID NO: 331 - protein, 9 aa, Homo sapiens
GSDKISRLW

SEQ ID NO: 332 - protein, 9 aa, Homo sapiens
GSDKSPRKL

SEQ ID NO: 333 - protein, 10 aa, Homo sapiens
GSDKSPRKLY

SEQ ID NO: 334 - protein, 11 aa, Homo sapiens
GSDRLWPVNGL

SEQ ID NO: 335 - protein, 9 aa, Homo sapiens
GSFLIGSSK

SEQ ID NO: 336 - protein, 9 aa, Homo sapiens
GSLKNKVQK

SEQ ID NO: 337 - protein, 9 aa, Homo sapiens
GSQLQKSTF

SEQ ID NO: 338 - protein, 9 aa, Homo sapiens
GSSNTSELK

SEQ ID NO: 339 - protein, 9 aa, Homo sapiens
GTAETYNSW

SEQ ID NO: 340 - protein, 10 aa, Homo sapiens
GTAETYNSWK

SEQ ID NO: 341 - protein, 9 aa, Homo sapiens
GTKLNVKLF

SEQ ID NO: 342 - protein, 9 aa, Homo sapiens
GTKLNVLLK

SEQ ID NO: 343 - protein, 9 aa, Homo sapiens
GYLRGAKKL

SEQ ID NO: 344 - protein, 9 aa, Homo sapiens
GYLSKVFSK

SEQ ID NO: 345 - protein, 10 aa, Homo sapiens
HEVNHSGKQA

SEQ ID NO: 346 - protein, 8 aa, Homo sapiens
HFFPSSHL

SEQ ID NO: 347 - protein, 9 aa, Homo sapiens
HFFPSSHLL

SEQ ID NO: 348 - protein, 10 aa, Homo sapiens
HFFPSSHLLK

SEQ ID NO: 349 - protein, 9 aa, Homo sapiens
HHFFPSSHL

SEQ ID NO: 350 - protein, 10 aa, Homo sapiens
HHFFPSSHLL

SEQ ID NO: 351 - protein, 9 aa, Homo sapiens
HKILKVSQL

SEQ ID NO: 352 - protein, 9 aa, Homo sapiens
HLLFGIYLL

SEQ ID NO: 353 - protein, 9 aa, Homo sapiens
HLLKLNVRK

SEQ ID NO: 354 - protein, 9 aa, Homo sapiens
HLSSEATKL

SEQ ID NO: 355 - protein, 10 aa, Homo sapiens
HPAKIKMQPL

SEQ ID NO: 356 - protein, 8 aa, Homo sapiens
HPGSFLDW

SEQ ID NO: 357 - protein, 9 aa, Homo sapiens
HPGSFLDWF

SEQ ID NO: 358 - protein, 10 aa, Homo sapiens
HPGSFLDWFF

SEQ ID NO: 359 - protein, 9 aa, Homo sapiens
HQAAGAWRV

SEQ ID NO: 360 - protein, 10 aa, Homo sapiens
HQAAGAWRVW

SEQ ID NO: 361 - protein, 9 aa, Homo sapiens
HSISTRQQL

SEQ ID NO: 362 - protein, 10 aa, Homo sapiens
HSMSPERNGK

SEQ ID NO: 363 - protein, 10 aa, Homo sapiens
HSRFQSASHL

SEQ ID NO: 364 - protein, 10 aa, Homo sapiens
HTHLAQGYLR

SEQ ID NO: 365 - protein, 9 aa, Homo sapiens
HVMVILQIK

SEQ ID NO: 366 - protein, 9 aa, Homo sapiens
HWDMKLTTV

SEQ ID NO: 367 - protein, 9 aa, Homo sapiens
IAAENQMTI

SEQ ID NO: 368 - protein, 10 aa, Homo sapiens
IAAENQMTIL

SEQ ID NO: 369 - protein, 9 aa, Homo sapiens
IAASHGFGK

SEQ ID NO: 370 - protein, 8 aa, Homo sapiens
IAPLLPSV

SEQ ID NO: 371 - protein, 9 aa, Homo sapiens
IAYSCDQNI

SEQ ID NO: 372 - protein, 9 aa, Homo sapiens
IENHNKCQL

SEQ ID NO: 373 - protein, 8 aa, Homo sapiens
IFTDSFSY

SEQ ID NO: 374 - protein, 9 aa, Homo sapiens
IHWDMKLTT

SEQ ID NO: 375 - protein, 10 aa, Homo sapiens
IHWDMKLTTV

SEQ ID NO: 376 - protein, 10 aa, Homo sapiens
IISDSSALEK

SEQ ID NO: 377 - protein, 10 aa, Homo sapiens
IISNQVILAK

SEQ ID NO: 378 - protein, 9 aa, Homo sapiens
IKEAASHGF

SEQ ID NO: 379 - protein, 8 aa, Homo sapiens
IKEKLQVM

SEQ ID NO: 380 - protein, 9 aa, Homo sapiens
IKLSISNSY

SEQ ID NO: 381 - protein, 9 aa, Homo sapiens
IKVSPYLSI

SEQ ID NO: 382 - protein, 9 aa, Homo sapiens
ILEESGSQL

SEQ ID NO: 383 - protein, 9 aa, Homo sapiens
ILHNSLEDY

SEQ ID NO: 384 - protein, 9 aa, Homo sapiens
ILKMKITNI

SEQ ID NO: 385 - protein, 10 aa, Homo sapiens
ILKMKITNIL

SEQ ID NO: 386 - protein, 9 aa, Homo sapiens
ILKVSQLVL

SEQ ID NO: 387 - protein, 9 aa, Homo sapiens
ILLALLLKK

SEQ ID NO: 388 - protein, 9 aa, Homo sapiens
ILVLVKVLL

SEQ ID NO: 389 - protein, 8 aa, Homo sapiens
IMNIPMEK

SEQ ID NO: 390 - protein, 9 aa, Homo sapiens
IMNQDVCAL

SEQ ID NO: 391 - protein, 10 aa, Homo sapiens
IMSGLEKVSK

SEQ ID NO: 392 - protein, 9 aa, Homo sapiens
INDMRFISL

SEQ ID NO: 393 - protein, 8 aa, Homo sapiens
IPLSLLGI

SEQ ID NO: 394 - protein, 9 aa, Homo sapiens
IPLSLLGIA

SEQ ID NO: 395 - protein, 8 aa, Homo sapiens
IPVNLKNV

SEQ ID NO: 396 - protein, 9 aa, Homo sapiens
IREAASHGF

SEQ ID NO: 397 - protein, 10 aa, Homo sapiens
IRKDHIGKSM

SEQ ID NO: 398 - protein, 9 aa, Homo sapiens
ISAHSGSLK

SEQ ID NO: 399 - protein, 10 aa, Homo sapiens
ISAHSGSLKK

SEQ ID NO: 400 - protein, 9 aa, Homo sapiens
ISDSSALEK

SEQ ID NO: 401 - protein, 9 aa, Homo sapiens
ISKEVVLTL

SEQ ID NO: 402 - protein, 10 aa, Homo sapiens
ISLFSDDLNL

SEQ ID NO: 403 - protein, 8 aa, Homo sapiens
ISLQAFLW

SEQ ID NO: 404 - protein, 9 aa, Homo sapiens
ISLTKTTAV

SEQ ID NO: 405 - protein, 9 aa, Homo sapiens
ISNQVILAK

SEQ ID NO: 406 - protein, 9 aa, Homo sapiens
ISRGIQNVK

SEQ ID NO: 407 - protein, 9 aa, Homo sapiens
ISRGKDKLK

SEQ ID NO: 408 - protein, 9 aa, Homo sapiens
ISRIIQNVK

SEQ ID NO: 409 - protein, 10 aa, Homo sapiens
ISRLWPVNGL

SEQ ID NO: 410 - protein, 9 aa, Homo sapiens
ISSNQAVAV

SEQ ID NO: 411 - protein, 10 aa, Homo sapiens
ISSRGTVVNK

SEQ ID NO: 412 - protein, 9 aa, Homo sapiens
ITLIAAFRK

SEQ ID NO: 413 - protein, 9 aa, Homo sapiens
ITLNEVDEY

SEQ ID NO: 414 - protein, 9 aa, Homo sapiens
ITRDRDKLK

SEQ ID NO: 415 - protein, 9 aa, Homo sapiens
ITREILKMK

SEQ ID NO: 416 - protein, 9 aa, Homo sapiens
IVKHKILKV

SEQ ID NO: 417 - protein, 9 aa, Homo sapiens
IVKHKSVPV

SEQ ID NO: 418 - protein, 9 aa, Homo sapiens
IVKIAKESL

SEQ ID NO: 419 - protein, 9 aa, Homo sapiens
IWFMFLQRR

SEQ ID NO: 420 - protein, 9 aa, Homo sapiens
IWKKSGNSF

SEQ ID NO: 421 - protein, 9 aa, Homo sapiens
IWKNIREFI

SEQ ID NO: 422 - protein, 9 aa, Homo sapiens
IWKNIRNSF

SEQ ID NO: 423 - protein, 9 aa, Homo sapiens
IWKNSGNSF

SEQ ID NO: 424 - protein, 9 aa, Homo sapiens
IWKTSGNSF

SEQ ID NO: 425 - protein, 9 aa, Homo sapiens
IYLLSLLGI

SEQ ID NO: 426 - protein, 9 aa, Homo sapiens
KAKTEPNTV

SEQ ID NO: 427 - protein, 9 aa, Homo sapiens
KALSIHWDM

SEQ ID NO: 428 - protein, 10 aa, Homo sapiens
KCQSGTAETY

SEQ ID NO: 429 - protein, 10 aa, Homo sapiens
KDIFTDSFSY

SEQ ID NO: 430 - protein, 10 aa, Homo sapiens
KDQRTYSIVK

SEQ ID NO: 431 - protein, 9 aa, Homo sapiens
KDVLSNLVI

SEQ ID NO: 432 - protein, 10 aa, Homo sapiens
KDVLSNLVMK

SEQ ID NO: 433 - protein, 8 aa, Homo sapiens
KEAASHGF

SEQ ID NO: 434 - protein, 10 aa, Homo sapiens
KEAASHGFGK

SEQ ID NO: 435 - protein, 8 aa, Homo sapiens
KEALGFVY

SEQ ID NO: 436 - protein, 9 aa, Homo sapiens
KEALGFVYH

SEQ ID NO: 437 - protein, 10 aa, Homo sapiens
KEALGFVYHL

SEQ ID NO: 438 - protein, 9 aa, Homo sapiens
KEDLSDLNF

SEQ ID NO: 439 - protein, 10 aa, Homo sapiens
KEDLSDLNFW

SEQ ID NO: 440 - protein, 10 aa, Homo sapiens
KEDLSEVAKA

SEQ ID NO: 441 - protein, 9 aa, Homo sapiens
KEEKEAAKY

SEQ ID NO: 442 - protein, 9 aa, Homo sapiens
KEELTLSGL

SEQ ID NO: 443 - protein, 9 aa, Homo sapiens
KEKLQVMDL

SEQ ID NO: 444 - protein, 8 aa, Homo sapiens
KEMGNENI

SEQ ID NO: 445 - protein, 9 aa, Homo sapiens
KENNENCSI

SEQ ID NO: 446 - protein, 8 aa, Homo sapiens
KEQGLTAA

SEQ ID NO: 447 - protein, 9 aa, Homo sapiens
KEQGLTAAV

SEQ ID NO: 448 - protein, 10 aa, Homo sapiens
KERIKPVQTV

SEQ ID NO: 449 - protein, 9 aa, Homo sapiens
KERMSLISS

SEQ ID NO: 450 - protein, 10 aa, Homo sapiens
KERMSLISSL

SEQ ID NO: 451 - protein, 9 aa, Homo sapiens
KESYKGNNY

SEQ ID NO: 452 - protein, 9 aa, Homo sapiens
KETDPNFKK

SEQ ID NO: 453 - protein, 10 aa, Homo sapiens
KETFNASFQV

SEQ ID NO: 454 - protein, 11 aa, Homo sapiens
KEVVPSLACEL

SEQ ID NO: 455 - protein, 9 aa, Homo sapiens
KEVVPVNGL

SEQ ID NO: 456 - protein, 10 aa, Homo sapiens
KEVVPVNGLL

SEQ ID NO: 457 - protein, 9 aa, Homo sapiens
KHVMVILQI

SEQ ID NO: 458 - protein, 9 aa, Homo sapiens
KIKERIKPV

SEQ ID NO: 459 - protein, 8 aa, Homo sapiens
KIKERMSL

SEQ ID NO: 460 - protein, 9 aa, Homo sapiens
KIKERMSLI

SEQ ID NO: 461 - protein, 9 aa, Homo sapiens
KILKVSQLV

SEQ ID NO: 462 - protein, 8 aa, Homo sapiens
KIMSGLEK

SEQ ID NO: 463 - protein, 9 aa, Homo sapiens
KIMSGLEKV

SEQ ID NO: 464 - protein, 11 aa, Homo sapiens
KIMSGLEKVSK

SEQ ID NO: 465 - protein, 9 aa, Homo sapiens
KISKEELTL

SEQ ID NO: 466 - protein, 9 aa, Homo sapiens
KISKEVVPV

SEQ ID NO: 467 - protein, 11 aa, Homo sapiens
KISRLWPVNGL

SEQ ID NO: 468 - protein, 10 aa, Homo sapiens
KITREILKMK

SEQ ID NO: 469 - protein, 9 aa, Homo sapiens
KIWFMFLQR

SEQ ID NO: 470 - protein, 9 aa, Homo sapiens
KKIKERMSL

SEQ ID NO: 471 - protein, 9 aa, Homo sapiens
KKNDSIYRF

SEQ ID NO: 472 - protein, 9 aa, Homo sapiens
KKVDLNAEW

SEQ ID NO: 473 - protein, 9 aa, Homo sapiens
KLDKFKLDL

SEQ ID NO: 474 - protein, 9 aa, Homo sapiens
KLIVVLAVK

SEQ ID NO: 475 - protein, 9 aa, Homo sapiens
KLKKHVMVI

SEQ ID NO: 476 - protein, 8 aa, Homo sapiens
KLKPALGR

SEQ ID NO: 477 - protein, 10 aa, Homo sapiens
KLKPALGRSW

SEQ ID NO: 478 - protein, 9 aa, Homo sapiens
KLLKITREI

SEQ ID NO: 479 - protein, 9 aa, Homo sapiens
KLMSGFPEV

SEQ ID NO: 480 - protein, 10 aa, Homo sapiens
KLMSGFPEVM

SEQ ID NO: 481 - protein, 8 aa, Homo sapiens
KLMSGFSR

SEQ ID NO: 482 - protein, 9 aa, Homo sapiens
KLNVLLKQK

SEQ ID NO: 483 - protein, 9 aa, Homo sapiens
KLQVMDLEK

SEQ ID NO: 484 - protein, 8 aa, Homo sapiens
KLRNTSNK

SEQ ID NO: 485 - protein, 8 aa, Homo sapiens
KLSISNSY

SEQ ID NO: 486 - protein, 10 aa, Homo sapiens
KLTNAPGLLL

SEQ ID NO: 487 - protein, 11 aa, Homo sapiens
KLYRLWPVNGL

SEQ ID NO: 488 - protein, 9 aa, Homo sapiens
KMKITNILL

SEQ ID NO: 489 - protein, 9 aa, Homo sapiens
KMQPLNCPY

SEQ ID NO: 490 - protein, 10 aa, Homo sapiens
KMQPLNCPYL

SEQ ID NO: 491 - protein, 9 aa, Homo sapiens
KMSKTSSKV

SEQ ID NO: 492 - protein, 10 aa, Homo sapiens
KNIRKDHIGK

SEQ ID NO: 493 - protein, 9 aa, Homo sapiens
KNKMDILSL

SEQ ID NO: 494 - protein, 9 aa, Homo sapiens
KNVNPSLPR

SEQ ID NO: 495 - protein, 11 aa, Homo sapiens
KPFEKKSPRKL

SEQ ID NO: 496 - protein, 10 aa, Homo sapiens
KPFESGSDRL

SEQ ID NO: 497 - protein, 11 aa, Homo sapiens
KPFESGSDRLW

SEQ ID NO: 498 - protein, 10 aa, Homo sapiens
KQDFNSNHNI

SEQ ID NO: 499 - protein, 9 aa, Homo sapiens
KQKAVKLFS

SEQ ID NO: 500 - protein, 10 aa, Homo sapiens
KRAASHGFGK

SEQ ID NO: 501 - protein, 9 aa, Homo sapiens
KRKKIKERM

SEQ ID NO: 502 - protein, 10 aa, Homo sapiens
KRLLQSDPSL

SEQ ID NO: 503 - protein, 11 aa, Homo sapiens
KSEKPLNEEQW

SEQ ID NO: 504 - protein, 8 aa, Homo sapiens
KSFVKTLR

SEQ ID NO: 505 - protein, 9 aa, Homo sapiens
KSLLNVNKR

SEQ ID NO: 506 - protein, 10 aa, Homo sapiens
KSLLNVNKRK

SEQ ID NO: 507 - protein, 9 aa, Homo sapiens
KSPRKLYRL

SEQ ID NO: 508 - protein, 10 aa, Homo sapiens
KSPRKLYRLW

SEQ ID NO: 509 - protein, 11 aa, Homo sapiens
KSVPVGTGNQL

SEQ ID NO: 510 - protein, 9 aa, Homo sapiens
KSWIDHIGK

SEQ ID NO: 511 - protein, 9 aa, Homo sapiens
KSWIRNSCK

SEQ ID NO: 512 - protein, 9 aa, Homo sapiens
KSWIWKNIK

SEQ ID NO: 513 - protein, 9 aa, Homo sapiens
KSWIWKNIR

SEQ ID NO: 514 - protein, 10 aa, Homo sapiens
KSWIWKNIRK

SEQ ID NO: 515 - protein, 10 aa, Homo sapiens
KSWIWKNISK

SEQ ID NO: 516 - protein, 9 aa, Homo sapiens
KSWIWNSFK

SEQ ID NO: 517 - protein, 11 aa, Homo sapiens
KTECFTEALQK

SEQ ID NO: 518 - protein, 9 aa, Homo sapiens
KTEPNTVNI

SEQ ID NO: 519 - protein, 9 aa, Homo sapiens
KTEPQSQLV

SEQ ID NO: 520 - protein, 9 aa, Homo sapiens
KTHIVYHHF

SEQ ID NO: 521 - protein, 9 aa, Homo sapiens
KTQNQREAA

SEQ ID NO: 522 - protein, 9 aa, Homo sapiens
KTSKSGKRR

SEQ ID NO: 523 - protein, 9 aa, Homo sapiens
KTSLKGNNY

SEQ ID NO: 524 - protein, 10 aa, Homo sapiens
KTSSDVELTK

SEQ ID NO: 525 - protein, 9 aa, Homo sapiens
KTSSKGNNY

SEQ ID NO: 526 - protein, 10 aa, Homo sapiens
KTSSKVELTK

SEQ ID NO: 527 - protein, 9 aa, Homo sapiens
KTSSKVTIM

SEQ ID NO: 528 - protein, 9 aa, Homo sapiens
KTSSKVTNI

SEQ ID NO: 529 - protein, 11 aa, Homo sapiens
KTSTLPFISLK

SEQ ID NO: 530 - protein, 9 aa, Homo sapiens
KTTAVAVTF

SEQ ID NO: 531 - protein, 9 aa, Homo sapiens
KTTPIKQQL

SEQ ID NO: 532 - protein, 9 aa, Homo sapiens
KTVKTQREA

SEQ ID NO: 533 - protein, 11 aa, Homo sapiens
KVDLNADPLCY

SEQ ID NO: 534 - protein, 8 aa, Homo sapiens
KVDLNAEW

SEQ ID NO: 535 - protein, 10 aa, Homo sapiens
KVDLNAEWNK

SEQ ID NO: 536 - protein, 8 aa, Homo sapiens
KVFSKVLF

SEQ ID NO: 537 - protein, 9 aa, Homo sapiens
KVFSKVLFK

SEQ ID NO: 538 - protein, 10 aa, Homo sapiens
KVKNLQLEPR

SEQ ID NO: 539 - protein, 9 aa, Homo sapiens
KVNNIPLSL

SEQ ID NO: 540 - protein, 10 aa, Homo sapiens
KVNNIPLSLL

SEQ ID NO: 541 - protein, 9 aa, Homo sapiens
KVQKSLLNV

SEQ ID NO: 542 - protein, 9 aa, Homo sapiens
KVRQVFSEI

SEQ ID NO: 543 - protein, 9 aa, Homo sapiens
KVSKRQSQK

SEQ ID NO: 544 - protein, 11 aa, Homo sapiens
KVSPYLSISTR

SEQ ID NO: 545 - protein, 9 aa, Homo sapiens
KVSQLVLEI

SEQ ID NO: 546 - protein, 11 aa, Homo sapiens
KVTIMNIPMEK

SEQ ID NO: 547 - protein, 9 aa, Homo sapiens
KVTIMNLML

SEQ ID NO: 548 - protein, 9 aa, Homo sapiens
KVTNIPMEK

SEQ ID NO: 549 - protein, 9 aa, Homo sapiens
KWSPDGENI

SEQ ID NO: 550 - protein, 9 aa, Homo sapiens
KYKHPGSFL

SEQ ID NO: 551 - protein, 10 aa, Homo sapiens
KYKHPGSFLI

SEQ ID NO: 552 - protein, 9 aa, Homo sapiens
LAKTSTLPF

SEQ ID NO: 553 - protein, 9 aa, Homo sapiens
LDNDDVARI

SEQ ID NO: 554 - protein, 9 aa, Homo sapiens
LDSPLRVEV

SEQ ID NO: 555 - protein, 9 aa, Homo sapiens
LEKHQGIHL

SEQ ID NO: 556 - protein, 10 aa, Homo sapiens
LEPRRVTSQM

SEQ ID NO: 557 - protein, 9 aa, Homo sapiens
LFSDDLNLI

SEQ ID NO: 558 - protein, 10 aa, Homo sapiens
LFSDDLNLIL

SEQ ID NO: 559 - protein, 9 aa, Homo sapiens
LGFSWTPSR

SEQ ID NO: 560 - protein, 9 aa, Homo sapiens
LHIAAENQM

SEQ ID NO: 561 - protein, 9 aa, Homo sapiens
LHNSVFADI

SEQ ID NO: 562 - protein, 9 aa, Homo sapiens
LIMPNVVSK

SEQ ID NO: 563 - protein, 9 aa, Homo sapiens
LISLQAFLW

SEQ ID NO: 564 - protein, 9 aa, Homo sapiens
LKALEDFTL

SEQ ID NO: 565 - protein, 9 aa, Homo sapiens
LKKNASHGF

SEQ ID NO: 566 - protein, 9 aa, Homo sapiens
LKKNEIYRF

SEQ ID NO: 567 - protein, 9 aa, Homo sapiens
LKMKITNIL

SEQ ID NO: 568 - protein, 8 aa, Homo sapiens
LKQKAVKL

SEQ ID NO: 569 - protein, 9 aa, Homo sapiens
LKQKAVKLF

SEQ ID NO: 570 - protein, 9 aa, Homo sapiens
LKTEPQSQL

SEQ ID NO: 571 - protein, 8 aa, Homo sapiens
LLFGIYLL

SEQ ID NO: 572 - protein, 9 aa, Homo sapiens
LLKITREIL

SEQ ID NO: 573 - protein, 9 aa, Homo sapiens
LLKLNVRKI

SEQ ID NO: 574 - protein, 9 aa, Homo sapiens
LLKQKAVKL

SEQ ID NO: 575 - protein, 9 aa, Homo sapiens
LLLKKLLKI

SEQ ID NO: 576 - protein, 9 aa, Homo sapiens
LLLSVQIPV

SEQ ID NO: 577 - protein, 9 aa, Homo sapiens
LLQINMDFY

SEQ ID NO: 578 - protein, 9 aa, Homo sapiens
LLRFQIQEM

SEQ ID NO: 579 - protein, 9 aa, Homo sapiens
LLSESPVVL

SEQ ID NO: 580 - protein, 9 aa, Homo sapiens
LMSGFPEVM

SEQ ID NO: 581 - protein, 9 aa, Homo sapiens
LNADPLCYR

SEQ ID NO: 582 - protein, 9 aa, Homo sapiens
LPCISLKAA

SEQ ID NO: 583 - protein, 10 aa, Homo sapiens
LPCISLKAAV

SEQ ID NO: 584 - protein, 11 aa, Homo sapiens
LPDSQQVSRRW

SEQ ID NO: 585 - protein, 8 aa, Homo sapiens
LPFISLKA

SEQ ID NO: 586 - protein, 9 aa, Homo sapiens
LPFISLKAA

SEQ ID NO: 587 - protein, 10 aa, Homo sapiens
LPFISLKAAV

SEQ ID NO: 588 - protein, 10 aa, Homo sapiens
LPRISSRGTV

SEQ ID NO: 589 - protein, 9 aa, Homo sapiens
LPSDAPGAL

SEQ ID NO: 590 - protein, 8 aa, Homo sapiens
LRFQIQEM

SEQ ID NO: 591 - protein, 10 aa, Homo sapiens
LRFQIQEMQK

SEQ ID NO: 592 - protein, 9 aa, Homo sapiens
LRKLKKHVM

SEQ ID NO: 593 - protein, 10 aa, Homo sapiens
LRRPNFTRRL

SEQ ID NO: 594 - protein, 10 aa, Homo sapiens
LRTNPFRSKW

SEQ ID NO: 595 - protein, 9 aa, Homo sapiens
LSDLNFWKL

SEQ ID NO: 596 - protein, 9 aa, Homo sapiens
LSDLNFWKY

SEQ ID NO: 597 - protein, 9 aa, Homo sapiens
LSICRIHSR

SEQ ID NO: 598 - protein, 9 aa, Homo sapiens
LSIHWDMKL

SEQ ID NO: 599 - protein, 9 aa, Homo sapiens
LSLLGIAPL

SEQ ID NO: 600 - protein, 10 aa, Homo sapiens
LSQFQQDNSW

SEQ ID NO: 601 - protein, 9 aa, Homo sapiens
LSVQIPVNL

SEQ ID NO: 602 - protein, 10 aa, Homo sapiens
LTAAVPISCK

SEQ ID NO: 603 - protein, 9 aa, Homo sapiens
LTKTTAVAV

SEQ ID NO: 604 - protein, 9 aa, Homo sapiens
LTKTTPITK

SEQ ID NO: 605 - protein, 9 aa, Homo sapiens
LTNAPGLLL

SEQ ID NO: 606 - protein, 9 aa, Homo sapiens
LTNQSPYSV

SEQ ID NO: 607 - protein, 8 aa, Homo sapiens
LVRKISQL

SEQ ID NO: 608 - protein, 9 aa, Homo sapiens
LVRKISQLI

SEQ ID NO: 609 - protein, 10 aa, Homo sapiens
LWPVNGLLEL

SEQ ID NO: 610 - protein, 9 aa, Homo sapiens
LYENNSNYY

SEQ ID NO: 611 - protein, 10 aa, Homo sapiens
LYRLWPVNGL

SEQ ID NO: 612 - protein, 8 aa, Homo sapiens
MDFYKTHI

SEQ ID NO: 613 - protein, 9 aa, Homo sapiens
MDFYKTHIV

SEQ ID NO: 614 - protein, 10 aa, Homo sapiens
MDFYKTHIVY

SEQ ID NO: 615 - protein, 9 aa, Homo sapiens
MDLEKHQGI

SEQ ID NO: 616 - protein, 9 aa, Homo sapiens
MEESELDAL

SEQ ID NO: 617 - protein, 9 aa, Homo sapiens
MFKIENHNK

SEQ ID NO: 618 - protein, 9 aa, Homo sapiens
MFLQRRTKI

SEQ ID NO: 619 - protein, 9 aa, Homo sapiens
MISRGIQNV

SEQ ID NO: 620 - protein, 10 aa, Homo sapiens
MISRGIQNVK

SEQ ID NO: 621 - protein, 9 aa, Homo sapiens
MISRIIQNV

SEQ ID NO: 622 - protein, 9 aa, Homo sapiens
MNRQAPGSF

SEQ ID NO: 623 - protein, 9 aa, Homo sapiens
MPNVVSKEA

SEQ ID NO: 624 - protein, 10 aa, Homo sapiens
MPNVVSKEAL

SEQ ID NO: 625 - protein, 9 aa, Homo sapiens
MRFINSNQA

SEQ ID NO: 626 - protein, 10 aa, Homo sapiens
MRFINSNQAV

SEQ ID NO: 627 - protein, 9 aa, Homo sapiens
MRFISLTKT

SEQ ID NO: 628 - protein, 9 aa, Homo sapiens
MRFISSNQA

SEQ ID NO: 629 - protein, 10 aa, Homo sapiens
MRFISSNQAV

SEQ ID NO: 630 - protein, 11 aa, Homo sapiens
MSKTSSKGNNY

SEQ ID NO: 631 - protein, 9 aa, Homo sapiens
MSLISSLYR

SEQ ID NO: 632 - protein, 9 aa, Homo sapiens
NAPGLLLSV

SEQ ID NO: 633 - protein, 8 aa, Homo sapiens
NDMRFISL

SEQ ID NO: 634 - protein, 10 aa, Homo sapiens
NDRFIASVPV

SEQ ID NO: 635 - protein, 10 aa, Homo sapiens
NDRFIASVTV

SEQ ID NO: 636 - protein, 10 aa, Homo sapiens
NEQKALSIHW

SEQ ID NO: 637 - protein, 9 aa, Homo sapiens
NEVGFRGSW

SEQ ID NO: 638 - protein, 10 aa, Homo sapiens
NFEDILHNSL

SEQ ID NO: 639 - protein, 9 aa, Homo sapiens
NFWKLRKLK

SEQ ID NO: 640 - protein, 10 aa, Homo sapiens
NHDESLKTQI

SEQ ID NO: 641 - protein, 9 aa, Homo sapiens
NHDESLKTV

SEQ ID NO: 642 - protein, 8 aa, Homo sapiens
NHDESLTV

SEQ ID NO: 643 - protein, 10 aa, Homo sapiens
NHITRDRDKL

SEQ ID NO: 644 - protein, 10 aa, Homo sapiens
NHNILQKSTF

SEQ ID NO: 645 - protein, 9 aa, Homo sapiens
NHNKCQLIL

SEQ ID NO: 646 - protein, 10 aa, Homo sapiens
NHSISTRQQL

SEQ ID NO: 647 - protein, 8 aa, Homo sapiens
NILQKSTF

SEQ ID NO: 648 - protein, 9 aa, Homo sapiens
NIREVNSCK

SEQ ID NO: 649 - protein, 9 aa, Homo sapiens
NIRKDHIGK

SEQ ID NO: 650 - protein, 9 aa, Homo sapiens
NISKVNSCK

SEQ ID NO: 651 - protein, 9 aa, Homo sapiens
NKDHIGKSM

SEQ ID NO: 652 - protein, 9 aa, Homo sapiens
NKEAASHGF

SEQ ID NO: 653 - protein, 8 aa, Homo sapiens
NKMDILSL

SEQ ID NO: 654 - protein, 9 aa, Homo sapiens
NKQKLPCSL

SEQ ID NO: 655 - protein, 9 aa, Homo sapiens
NLKNVNPSL

SEQ ID NO: 656 - protein, 9 aa, Homo sapiens
NLLTNQSPY

SEQ ID NO: 657 - protein, 10 aa, Homo sapiens
NLPSDAPGAL

SEQ ID NO: 658 - protein, 9 aa, Homo sapiens
NMDFYKTHI

SEQ ID NO: 659 - protein, 11 aa, Homo sapiens
NMDFYKTHIVY

SEQ ID NO: 660 - protein, 9 aa, Homo sapiens
NNFEVGPPV

SEQ ID NO: 661 - protein, 10 aa, Homo sapiens
NPFRLNGAQM

SEQ ID NO: 662 - protein, 10 aa, Homo sapiens
NPFRSKGAQM

SEQ ID NO: 663 - protein, 10 aa, Homo sapiens
NPFRSKWAQM

SEQ ID NO: 664 - protein, 8 aa, Homo sapiens
NQAHLIVL

SEQ ID NO: 665 - protein, 9 aa, Homo sapiens
NQRSHGFGK

SEQ ID NO: 666 - protein, 9 aa, Homo sapiens
NQRTASHGF

SEQ ID NO: 667 - protein, 9 aa, Homo sapiens
NREAASHGF

SEQ ID NO: 668 - protein, 8 aa, Homo sapiens
NRNEQKAL

SEQ ID NO: 669 - protein, 9 aa, Homo sapiens
NRQVKDMTA

SEQ ID NO: 670 - protein, 9 aa, Homo sapiens
NSLPRISSR

SEQ ID NO: 671 - protein, 9 aa, Homo sapiens
NSNHNILQK

SEQ ID NO: 672 - protein, 9 aa, Homo sapiens
NTASHGFGK

SEQ ID NO: 673 - protein, 9 aa, Homo sapiens
NTIKEAASH

SEQ ID NO: 674 - protein, 9 aa, Homo sapiens
NTKRSCKSW

SEQ ID NO: 675 - protein, 9 aa, Homo sapiens
NTNQRGFGK

SEQ ID NO: 676 - protein, 9 aa, Homo sapiens
NTNQRSCKY

SEQ ID NO: 677 - protein, 9 aa, Homo sapiens
NTNQRSHGF

SEQ ID NO: 678 - protein, 9 aa, Homo sapiens
NTNSSIQKV

SEQ ID NO: 679 - protein, 9 aa, Homo sapiens
NTVNITAGF

SEQ ID NO: 680 - protein, 10 aa, Homo sapiens
NVANQKPFEK

SEQ ID NO: 681 - protein, 9 aa, Homo sapiens
NVANQKPLR

SEQ ID NO: 682 - protein, 10 aa, Homo sapiens
NVAQSPRKLY

SEQ ID NO: 683 - protein, 11 aa, Homo sapiens
NVAQSPRKLYR

SEQ ID NO: 684 - protein, 9 aa, Homo sapiens
NVEKETAKR

SEQ ID NO: 685 - protein, 10 aa, Homo sapiens
NVGSPLRVEV

SEQ ID NO: 686 - protein, 9 aa, Homo sapiens
NVLLKQKAV

SEQ ID NO: 687 - protein, 8 aa, Homo sapiens
NVNKRKKI

SEQ ID NO: 688 - protein, 9 aa, Homo sapiens
NVQHSLPTL

SEQ ID NO: 689 - protein, 8 aa, Homo sapiens
NVVSKEAL

SEQ ID NO: 690 - protein, 10 aa, Homo sapiens
NVVSKEALGF

SEQ ID NO: 691 - protein, 10 aa, Homo sapiens
NYFETEAVAF

SEQ ID NO: 692 - protein, 10 aa, Homo sapiens
NYLYENNSNY

SEQ ID NO: 693 - protein, 9 aa, Homo sapiens
PAKIKMQPL

SEQ ID NO: 694 - protein, 8 aa, Homo sapiens
PMEKIPLL

SEQ ID NO: 695 - protein, 9 aa, Homo sapiens
PWITLIAAF

SEQ ID NO: 696 - protein, 10 aa, Homo sapiens
PYSVVNSSAF

SEQ ID NO: 697 - protein, 9 aa, Homo sapiens
QAAGAWRVW

SEQ ID NO: 698 - protein, 9 aa, Homo sapiens
QAFLWLVRK

SEQ ID NO: 699 - protein, 9 aa, Homo sapiens
QDFNSNHNI

SEQ ID NO: 700 - protein, 9 aa, Homo sapiens
QEHQAAGAW

SEQ ID NO: 701 - protein, 9 aa, Homo sapiens
QFQQDNSWY

SEQ ID NO: 702 - protein, 9 aa, Homo sapiens
QGYLRGAKK

SEQ ID NO: 703 - protein, 9 aa, Homo sapiens
QHLLFGIYL

SEQ ID NO: 704 - protein, 10 aa, Homo sapiens
QHLLFGIYLL

SEQ ID NO: 705 - protein, 9 aa, Homo sapiens
QIKEDLSAF

SEQ ID NO: 706 - protein, 9 aa, Homo sapiens
QIKEDLSEV

SEQ ID NO: 707 - protein, 8 aa, Homo sapiens
QIKEKLQV

SEQ ID NO: 708 - protein, 9 aa, Homo sapiens
QIKEKLQVM

SEQ ID NO: 709 - protein, 9 aa, Homo sapiens
QLEPRRVNK

SEQ ID NO: 710 - protein, 10 aa, Homo sapiens
QLIMPNVVSK

SEQ ID NO: 711 - protein, 9 aa, Homo sapiens
QLISLQAFL

SEQ ID NO: 712 - protein, 9 aa, Homo sapiens
QLKKIWFMF

SEQ ID NO: 713 - protein, 9 aa, Homo sapiens
QLQKSTFEV

SEQ ID NO: 714 - protein, 9 aa, Homo sapiens
QLVLATYHL

SEQ ID NO: 715 - protein, 10 aa, Homo sapiens
QMNRQAPGSF

SEQ ID NO: 716 - protein, 9 aa, Homo sapiens
QNMWRPNKR

SEQ ID NO: 717 - protein, 9 aa, Homo sapiens
QPLNCPPAF

SEQ ID NO: 718 - protein, 9 aa, Homo sapiens
QPLNCPYLI

SEQ ID NO: 719 - protein, 10 aa, Homo sapiens
QRTASHGFGK

SEQ ID NO: 720 - protein, 10 aa, Homo sapiens
QRTYSIGFSY

SEQ ID NO: 721 - protein, 9 aa, Homo sapiens
QRTYSIVKI

SEQ ID NO: 722 - protein, 9 aa, Homo sapiens
QSASHLLRF

SEQ ID NO: 723 - protein, 11 aa, Homo sapiens
QSGTAETYNSW

SEQ ID NO: 724 - protein, 10 aa, Homo sapiens
QSPRKLYRLW

SEQ ID NO: 725 - protein, 9 aa, Homo sapiens
QSQLVLATY

SEQ ID NO: 726 - protein, 9 aa, Homo sapiens
QSTVSTISR

SEQ ID NO: 727 - protein, 10 aa, Homo sapiens
QTEQASVNTV

SEQ ID NO: 728 - protein, 10 aa, Homo sapiens
QTESKSIFLK

SEQ ID NO: 729 - protein, 10 aa, Homo sapiens
QTESQNIKKY

SEQ ID NO: 730 - protein, 9 aa, Homo sapiens
QVKDMTAIL

SEQ ID NO: 731 - protein, 9 aa, Homo sapiens
RAASHGFGK

SEQ ID NO: 732 - protein, 9 aa, Homo sapiens
RAQMEKIPL

SEQ ID NO: 733 - protein, 10 aa, Homo sapiens
RAQMEKIPLL

SEQ ID NO: 734 - protein, 8 aa, Homo sapiens
REILKMKI

SEQ ID NO: 735 - protein, 9 aa, Homo sapiens
REILKMKIT

SEQ ID NO: 736 - protein, 8 aa, Homo sapiens
REQEHQAA

SEQ ID NO: 737 - protein, 9 aa, Homo sapiens
REQEHQAAG

SEQ ID NO: 738 - protein, 10 aa, Homo sapiens
REQEHQAAGA

SEQ ID NO: 739 - protein, 11 aa, Homo sapiens
REQEHQAAGAW

SEQ ID NO: 740 - protein, 9 aa, Homo sapiens
REYSCKDHI

SEQ ID NO: 741 - protein, 9 aa, Homo sapiens
RFIAASHGF

SEQ ID NO: 742 - protein, 9 aa, Homo sapiens
RFIASVPVK

SEQ ID NO: 743 - protein, 9 aa, Homo sapiens
RFIASVTEA

SEQ ID NO: 744 - protein, 9 aa, Homo sapiens
RFIASVTVK

SEQ ID NO: 745 - protein, 9 aa, Homo sapiens
RFINSNQAV

SEQ ID NO: 746 - protein, 9 aa, Homo sapiens
RFISSNQAV

SEQ ID NO: 747 - protein, 9 aa, Homo sapiens
RFKNNSNQA

SEQ ID NO: 748 - protein, 9 aa, Homo sapiens
RFQIQEMQK

SEQ ID NO: 749 - protein, 10 aa, Homo sapiens
RFQIQEMQKR

SEQ ID NO: 750 - protein, 9 aa, Homo sapiens
RFQSASHLL

SEQ ID NO: 751 - protein, 10 aa, Homo sapiens
RFQSASHLLR

SEQ ID NO: 752 - protein, 11 aa, Homo sapiens
RFQSASHLLRF

SEQ ID NO: 753 - protein, 9 aa, Homo sapiens
RGFYSALQK

SEQ ID NO: 754 - protein, 9 aa, Homo sapiens
RGKESYKLK

SEQ ID NO: 755 - protein, 10 aa, Homo sapiens
RGKESYKMSK

SEQ ID NO: 756 - protein, 10 aa, Homo sapiens
RIASGNKKVK

SEQ ID NO: 757 - protein, 9 aa, Homo sapiens
RIHSRFQSA

SEQ ID NO: 758 - protein, 9 aa, Homo sapiens
RIIQNVKDK

SEQ ID NO: 759 - protein, 9 aa, Homo sapiens
RIKPVQTVN

SEQ ID NO: 760 - protein, 10 aa, Homo sapiens
RIKPVQTVNI

SEQ ID NO: 761 - protein, 9 aa, Homo sapiens
RISSRGTVV

SEQ ID NO: 762 - protein, 11 aa, Homo sapiens
RISSRGTVVNK

SEQ ID NO: 763 - protein, 9 aa, Homo sapiens
RKDHIGKSM

SEQ ID NO: 764 - protein, 10 aa, Homo sapiens
RKEEKEAAKY

SEQ ID NO: 765 - protein, 10 aa, Homo sapiens
RKLMSGFPEV

SEQ ID NO: 766 - protein, 9 aa, Homo sapiens
RKNQEEQSM

SEQ ID NO: 767 - protein, 9 aa, Homo sapiens
RLLQSDPSL

SEQ ID NO: 768 - protein, 9 aa, Homo sapiens
RLNGAQMEK

SEQ ID NO: 769 - protein, 8 aa, Homo sapiens
RLWPVNGL

SEQ ID NO: 770 - protein, 9 aa, Homo sapiens
RLWPVNGLL

SEQ ID NO: 771 - protein, 11 aa, Homo sapiens
RLWPVNGLLEL

SEQ ID NO: 772 - protein, 9 aa, Homo sapiens
RMSLISSLY

SEQ ID NO: 773 - protein, 10 aa, Homo sapiens
RMSLISSLYR

SEQ ID NO: 774 - protein, 9 aa, Homo sapiens
RNQAHLIVL

SEQ ID NO: 775 - protein, 8 aa, Homo sapiens
RPNFTRRL

SEQ ID NO: 776 - protein, 10 aa, Homo sapiens
RPNFTRRLQF

SEQ ID NO: 777 - protein, 9 aa, Homo sapiens
RQAPGSFTK

SEQ ID NO: 778 - protein, 9 aa, Homo sapiens
RQLISLQAF

SEQ ID NO: 779 - protein, 10 aa, Homo sapiens
RQLISLQAFL

SEQ ID NO: 780 - protein, 9 aa, Homo sapiens
RQQLVLGTK

SEQ ID NO: 781 - protein, 9 aa, Homo sapiens
RQTESKSIF

SEQ ID NO: 782 - protein, 10 aa, Homo sapiens
RQTESKSIFL

SEQ ID NO: 783 - protein, 11 aa, Homo sapiens
RQTESQNIKKY

SEQ ID NO: 784 - protein, 9 aa, Homo sapiens
RQVKDMTAI

SEQ ID NO: 785 - protein, 10 aa, Homo sapiens
RQVKDMTAIL

SEQ ID NO: 786 - protein, 9 aa, Homo sapiens
RRLQFLYRL

SEQ ID NO: 787 - protein, 9 aa, Homo sapiens
RRPNFTRRL

SEQ ID NO: 788 - protein, 11 aa, Homo sapiens
RRPNFTRRLQF

SEQ ID NO: 789 - protein, 9 aa, Homo sapiens
RRVTSQMNR

SEQ ID NO: 790 - protein, 9 aa, Homo sapiens
RSCKSWNSF

SEQ ID NO: 791 - protein, 10 aa, Homo sapiens
RSCKSWNSFK

SEQ ID NO: 792 - protein, 9 aa, Homo sapiens
RSCKYGFGK

SEQ ID NO: 793 - protein, 9 aa, Homo sapiens
RSHGFGKTS

SEQ ID NO: 794 - protein, 9 aa, Homo sapiens
RSKGAQMEK

SEQ ID NO: 795 - protein, 9 aa, Homo sapiens
RSKWAQMEK

SEQ ID NO: 796 - protein, 9 aa, Homo sapiens
RSKWSPDGK

SEQ ID NO: 797 - protein, 9 aa, Homo sapiens
RSKWSPMEK

SEQ ID NO: 798 - protein, 9 aa, Homo sapiens
RSKWSQMEK

SEQ ID NO: 799 - protein, 9 aa, Homo sapiens
RTASHGFGK

SEQ ID NO: 800 - protein, 9 aa, Homo sapiens
RTNPFRLNG

SEQ ID NO: 801 - protein, 10 aa, Homo sapiens
RTNPFRLNGA

SEQ ID NO: 802 - protein, 8 aa, Homo sapiens
RTNPFRSK

SEQ ID NO: 803 - protein, 9 aa, Homo sapiens
RTNPFRSKG

SEQ ID NO: 804 - protein, 10 aa, Homo sapiens
RTNPFRSKGA

SEQ ID NO: 805 - protein, 9 aa, Homo sapiens
RTNPFRSKW

SEQ ID NO: 806 - protein, 10 aa, Homo sapiens
RTNPFRSKWA

SEQ ID NO: 807 - protein, 9 aa, Homo sapiens
RTQENLLSL

SEQ ID NO: 808 - protein, 10 aa, Homo sapiens
RTQENLLSLK

SEQ ID NO: 809 - protein, 9 aa, Homo sapiens
RTYSIGFSY

SEQ ID NO: 810 - protein, 10 aa, Homo sapiens
RTYSIGFSYS

SEQ ID NO: 811 - protein, 8 aa, Homo sapiens
RTYSIVKI

SEQ ID NO: 812 - protein, 9 aa, Homo sapiens
RTYSIVKIA

SEQ ID NO: 813 - protein, 10 aa, Homo sapiens
RTYSIVKIAK

SEQ ID NO: 814 - protein, 9 aa, Homo sapiens
RVEVTKSPR

SEQ ID NO: 815 - protein, 10 aa, Homo sapiens
RVEVTKSPRK

SEQ ID NO: 816 - protein, 9 aa, Homo sapiens
RVHSKSVEK

SEQ ID NO: 817 - protein, 11 aa, Homo sapiens
RVNKPNEQTSK

SEQ ID NO: 818 - protein, 9 aa, Homo sapiens
SAFMEDDEL

SEQ ID NO: 819 - protein, 9 aa, Homo sapiens
SAHGTKQCF

SEQ ID NO: 820 - protein, 10 aa, Homo sapiens
SAHSGSLKNK

SEQ ID NO: 821 - protein, 8 aa, Homo sapiens
SALEKAVL

SEQ ID NO: 822 - protein, 9 aa, Homo sapiens
SALQKAVKL

SEQ ID NO: 823 - protein, 9 aa, Homo sapiens
SDDEERNGL

SEQ ID NO: 824 - protein, 9 aa, Homo sapiens
SDDLNLILL

SEQ ID NO: 825 - protein, 8 aa, Homo sapiens
SDLNFWKL

SEQ ID NO: 826 - protein, 8 aa, Homo sapiens
SDLNFWKY

SEQ ID NO: 827 - protein, 10 aa, Homo sapiens
SDPSLQETSL

SEQ ID NO: 828 - protein, 10 aa, Homo sapiens
SDRLWPVNGL

SEQ ID NO: 829 - protein, 10 aa, Homo sapiens
SDSSALEDLW

SEQ ID NO: 830 - protein, 9 aa, Homo sapiens
SEATKLDSL

SEQ ID NO: 831 - protein, 10 aa, Homo sapiens
SEATKLDSLL

SEQ ID NO: 832 - protein, 10 aa, Homo sapiens
SEDILHNSLV

SEQ ID NO: 833 - protein, 9 aa, Homo sapiens
SEDILHNSV

SEQ ID NO: 834 - protein, 10 aa, Homo sapiens
SEDILHNSVF

SEQ ID NO: 835 - protein, 10 aa, Homo sapiens
SEFGRLDCKY

SEQ ID NO: 836 - protein, 10 aa, Homo sapiens
SEKPLNEEQW

SEQ ID NO: 837 - protein, 8 aa, Homo sapiens
SELDALSI

SEQ ID NO: 838 - protein, 9 aa, Homo sapiens
SELDALSIC

SEQ ID NO: 839 - protein, 11 aa, Homo sapiens
SENKEAASHGF

SEQ ID NO: 840 - protein, 9 aa, Homo sapiens
SENNHSIST

SEQ ID NO: 841 - protein, 11 aa, Homo sapiens
SENREAASHGF

SEQ ID NO: 842 - protein, 9 aa, Homo sapiens
SENTASHGF

SEQ ID NO: 843 - protein, 11 aa, Homo sapiens
SENTASHGFGK

SEQ ID NO: 844 - protein, 8 aa, Homo sapiens
SENTIKEA

SEQ ID NO: 845 - protein, 9 aa, Homo sapiens
SENTIKEAA

SEQ ID NO: 846 - protein, 11 aa, Homo sapiens
SENTKRSCKSW

SEQ ID NO: 847 - protein, 10 aa, Homo sapiens
SENTKRSHGF

SEQ ID NO: 848 - protein, 8 aa, Homo sapiens
SENTNHGF

SEQ ID NO: 849 - protein, 9 aa, Homo sapiens
SENTNKEAA

SEQ ID NO: 850 - protein, 9 aa, Homo sapiens
SENTNQRGF

SEQ ID NO: 851 - protein, 11 aa, Homo sapiens
SENTNQRSCKY

SEQ ID NO: 852 - protein, 11 aa, Homo sapiens
SENTNQRSHGF

SEQ ID NO: 853 - protein, 9 aa, Homo sapiens
SENTNQRTA

SEQ ID NO: 854 - protein, 9 aa, Homo sapiens
SETQIKEAA

SEQ ID NO: 855 - protein, 9 aa, Homo sapiens
SEVAKAQEA

SEQ ID NO: 856 - protein, 9 aa, Homo sapiens
SFDTTEHCV

SEQ ID NO: 857 - protein, 9 aa, Homo sapiens
SFQVSKSLY

SEQ ID NO: 858 - protein, 9 aa, Homo sapiens
SFSKVRQVF

SEQ ID NO: 859 - protein, 9 aa, Homo sapiens
SGFSRSDEL

SEQ ID NO: 860 - protein, 9 aa, Homo sapiens
SGKRRGEPL

SEQ ID NO: 861 - protein, 9 aa, Homo sapiens
SGSDKISRL

SEQ ID NO: 862 - protein, 10 aa, Homo sapiens
SGSDKISRLW

SEQ ID NO: 863 - protein, 10 aa, Homo sapiens
SGTAETYNSW

SEQ ID NO: 864 - protein, 10 aa, Homo sapiens
SHLLKLNVRK

SEQ ID NO: 865 - protein, 8 aa, Homo sapiens
SISTRQQL

SEQ ID NO: 866 - protein, 9 aa, Homo sapiens
SISTRQTTV

SEQ ID NO: 867 - protein, 9 aa, Homo sapiens
SKEALGFVY

SEQ ID NO: 868 - protein, 10 aa, Homo sapiens
SKEVVPVNGL

SEQ ID NO: 869 - protein, 10 aa, Homo sapiens
SKTSLKGNNY

SEQ ID NO: 870 - protein, 9 aa, Homo sapiens
SKTSSDVEL

SEQ ID NO: 871 - protein, 10 aa, Homo sapiens
SKTSSKGNNY

SEQ ID NO: 872 - protein, 9 aa, Homo sapiens
SKTSSKVEL

SEQ ID NO: 873 - protein, 9 aa, Homo sapiens
SKVFSKVLF

SEQ ID NO: 874 - protein, 10 aa, Homo sapiens
SKVFSKVLFK

SEQ ID NO: 875 - protein, 10 aa, Homo sapiens
SKVTNIPMEK

SEQ ID NO: 876 - protein, 9 aa, Homo sapiens
SKWSPMEKI

SEQ ID NO: 877 - protein, 9 aa, Homo sapiens
SLDDSEDIL

SEQ ID NO: 878 - protein, 10 aa, Homo sapiens
SLDNDDVARI

SEQ ID NO: 879 - protein, 9 aa, Homo sapiens
SLDNTNSSI

SEQ ID NO: 880 - protein, 9 aa, Homo sapiens
SLFSDDLNL

SEQ ID NO: 881 - protein, 10 aa, Homo sapiens
SLFSDDLNLI

SEQ ID NO: 882 - protein, 11 aa, Homo sapiens
SLFSDDLNLIL

SEQ ID NO: 883 - protein, 10 aa, Homo sapiens
SLKKNASHGF

SEQ ID NO: 884 - protein, 9 aa, Homo sapiens
SLKKNDSIY

SEQ ID NO: 885 - protein, 10 aa, Homo sapiens
SLKKNDSIYR

SEQ ID NO: 886 - protein, 9 aa, Homo sapiens
SLKKNEIYR

SEQ ID NO: 887 - protein, 10 aa, Homo sapiens
SLKNKVQKSL

SEQ ID NO: 888 - protein, 9 aa, Homo sapiens
SLKTVKTQR

SEQ ID NO: 889 - protein, 9 aa, Homo sapiens
SLLGIAPLL

SEQ ID NO: 890 - protein, 9 aa, Homo sapiens
SLLNVNKRK

SEQ ID NO: 891 - protein, 10 aa, Homo sapiens
SLLNVNKRKK

SEQ ID NO: 892 - protein, 9 aa, Homo sapiens
SLLQINMDF

SEQ ID NO: 893 - protein, 10 aa, Homo sapiens
SLLQINMDFY

SEQ ID NO: 894 - protein, 9 aa, Homo sapiens
SLQAFLWLV

SEQ ID NO: 895 - protein, 9 aa, Homo sapiens
SLRTNPFRL

SEQ ID NO: 896 - protein, 10 aa, Homo sapiens
SLRTNPFRSK

SEQ ID NO: 897 - protein, 11 aa, Homo sapiens
SLRTNPFRSKW

SEQ ID NO: 898 - protein, 9 aa, Homo sapiens
SLTKTTPIK

SEQ ID NO: 899 - protein, 10 aa, Homo sapiens
SLTKTTPITK

SEQ ID NO: 900 - protein, 9 aa, Homo sapiens
SLTVKTQIK

SEQ ID NO: 901 - protein, 10 aa, Homo sapiens
SLVCSTHSHK

SEQ ID NO: 902 - protein, 9 aa, Homo sapiens
SLYRQLISL

SEQ ID NO: 903 - protein, 10 aa, Homo sapiens
SMFKIENHNK

SEQ ID NO: 904 - protein, 9 aa, Homo sapiens
SMSPERNGK

SEQ ID NO: 905 - protein, 9 aa, Homo sapiens
SNHDESLTV

SEQ ID NO: 906 - protein, 11 aa, Homo sapiens
SNLPSDAPGAL

SEQ ID NO: 907 - protein, 9 aa, Homo sapiens
SNLVMISRI

SEQ ID NO: 908 - protein, 9 aa, Homo sapiens
SPDGEKDLL

SEQ ID NO: 909 - protein, 9 aa, Homo sapiens
SPDGENTPI

SEQ ID NO: 910 - protein, 11 aa, Homo sapiens
SPDGENTPIAY

SEQ ID NO: 911 - protein, 8 aa, Homo sapiens
SPDGKIPL

SEQ ID NO: 912 - protein, 9 aa, Homo sapiens
SPDGKIPLL

SEQ ID NO: 913 - protein, 9 aa, Homo sapiens
SPEMGNENI

SEQ ID NO: 914 - protein, 9 aa, Homo sapiens
SPKETDPNF

SEQ ID NO: 915 - protein, 8 aa, Homo sapiens
SPMEKIPL

SEQ ID NO: 916 - protein, 9 aa, Homo sapiens
SPMEKIPLL

SEQ ID NO: 917 - protein, 11 aa, Homo sapiens
SPMEKIPLLHI

SEQ ID NO: 918 - protein, 8 aa, Homo sapiens
SPRKLYRL

SEQ ID NO: 919 - protein, 9 aa, Homo sapiens
SPRKLYRLW

SEQ ID NO: 920 - protein, 9 aa, Homo sapiens
SPVASSFQV

SEQ ID NO: 921 - protein, 8 aa, Homo sapiens
SPYLSIST

SEQ ID NO: 922 - protein, 9 aa, Homo sapiens
SPYLSISTR

SEQ ID NO: 923 - protein, 9 aa, Homo sapiens
SPYSVVNSS

SEQ ID NO: 924 - protein, 10 aa, Homo sapiens
SPYSVVNSSA

SEQ ID NO: 925 - protein, 11 aa, Homo sapiens
SPYSVVNSSAF

SEQ ID NO: 926 - protein, 9 aa, Homo sapiens
SQFQQDNSW

SEQ ID NO: 927 - protein, 10 aa, Homo sapiens
SQFQQDNSWY

SEQ ID NO: 928 - protein, 8 aa, Homo sapiens
SQLIMPNV

SEQ ID NO: 929 - protein, 9 aa, Homo sapiens
SQLIMPNVV

SEQ ID NO: 930 - protein, 11 aa, Homo sapiens
SQLIMPNVVSK

SEQ ID NO: 931 - protein, 8 aa, Homo sapiens
SQLQKSTF

SEQ ID NO: 932 - protein, 8 aa, Homo sapiens
SQLVLATY

SEQ ID NO: 933 - protein, 8 aa, Homo sapiens
SQMEKIPL

SEQ ID NO: 934 - protein, 9 aa, Homo sapiens
SQMEKIPLL

SEQ ID NO: 935 - protein, 11 aa, Homo sapiens
SQMNRQAPGSF

SEQ ID NO: 936 - protein, 8 aa, Homo sapiens
SQNIKKYL

SEQ ID NO: 937 - protein, 9 aa, Homo sapiens
SQNIKKYLF

SEQ ID NO: 938 - protein, 11 aa, Homo sapiens
SRAQMEKIPLL

SEQ ID NO: 939 - protein, 9 aa, Homo sapiens
SRFQSASHL

SEQ ID NO: 940 - protein, 10 aa, Homo sapiens
SRFQSASHLL

SEQ ID NO: 941 - protein, 11 aa, Homo sapiens
SRFQSASHLLR

SEQ ID NO: 942 - protein, 9 aa, Homo sapiens
SRGKESYKL

SEQ ID NO: 943 - protein, 9 aa, Homo sapiens
SRLWPVNGL

SEQ ID NO: 944 - protein, 10 aa, Homo sapiens
SRLWPVNGLL

SEQ ID NO: 945 - protein, 9 aa, Homo sapiens
SRNQAHLIV

SEQ ID NO: 946 - protein, 10 aa, Homo sapiens
SRNQAHLIVL

SEQ ID NO: 947 - protein, 8 aa, Homo sapiens
SSALEDLW

SEQ ID NO: 948 - protein, 9 aa, Homo sapiens
SSFQVSKSL

SEQ ID NO: 949 - protein, 10 aa, Homo sapiens
SSFQVSKSLY

SEQ ID NO: 950 - protein, 9 aa, Homo sapiens
SSGLYIFRK

SEQ ID NO: 951 - protein, 9 aa, Homo sapiens
SSKVTIMNI

SEQ ID NO: 952 - protein, 9 aa, Homo sapiens
SSKVTIMNL

SEQ ID NO: 953 - protein, 9 aa, Homo sapiens
SSKVTNIPM

SEQ ID NO: 954 - protein, 11 aa, Homo sapiens
SSKVTNIPMEK

SEQ ID NO: 955 - protein, 10 aa, Homo sapiens
SSNQAVAVTF

SEQ ID NO: 956 - protein, 9 aa, Homo sapiens
SSRGTVVNK

SEQ ID NO: 957 - protein, 10 aa, Homo sapiens
SSRGTVVNKR

SEQ ID NO: 958 - protein, 9 aa, Homo sapiens
STLPCISLK

SEQ ID NO: 959 - protein, 9 aa, Homo sapiens
STLPFISLK

SEQ ID NO: 960 - protein, 8 aa, Homo sapiens
STRQQLVL

SEQ ID NO: 961 - protein, 9 aa, Homo sapiens
STRQTTVGI

SEQ ID NO: 962 - protein, 10 aa, Homo sapiens
STRQTTVGIR

SEQ ID NO: 963 - protein, 9 aa, Homo sapiens
STVATECLR

SEQ ID NO: 964 - protein, 11 aa, Homo sapiens
STYSKDSENAK

SEQ ID NO: 965 - protein, 9 aa, Homo sapiens
SVNTVSAHL

SEQ ID NO: 966 - protein, 9 aa, Homo sapiens
SVQIPVNLK

SEQ ID NO: 967 - protein, 10 aa, Homo sapiens
SVTDSENTIK

SEQ ID NO: 968 - protein, 9 aa, Homo sapiens
SVTDSENTK

SEQ ID NO: 969 - protein, 10 aa, Homo sapiens
SVTDSENTKR

SEQ ID NO: 970 - protein, 10 aa, Homo sapiens
SVTDSENTNK

SEQ ID NO: 971 - protein, 10 aa, Homo sapiens
SVTDSETQIK

SEQ ID NO: 972 - protein, 10 aa, Homo sapiens
SVTEAASHGF

SEQ ID NO: 973 - protein, 10 aa, Homo sapiens
SVTVKHTNQR

SEQ ID NO: 974 - protein, 9 aa, Homo sapiens
SVTVKTNQR

SEQ ID NO: 975 - protein, 9 aa, Homo sapiens
SVTVKTQIK

SEQ ID NO: 976 - protein, 9 aa, Homo sapiens
SVTVKTQIR

SEQ ID NO: 977 - protein, 10 aa, Homo sapiens
SVTVKTQNQR

SEQ ID NO: 978 - protein, 10 aa, Homo sapiens
SWIDHIGKSM

SEQ ID NO: 979 - protein, 11 aa, Homo sapiens
SWIWKKSGNSF

SEQ ID NO: 980 - protein, 9 aa, Homo sapiens
SWIWKNIKV

SEQ ID NO: 981 - protein, 10 aa, Homo sapiens
SWIWKNIREF

SEQ ID NO: 982 - protein, 9 aa, Homo sapiens
SWIWKNIRK

SEQ ID NO: 983 - protein, 11 aa, Homo sapiens
SWIWKNIRNSF

SEQ ID NO: 984 - protein, 9 aa, Homo sapiens
SWIWKNISK

SEQ ID NO: 985 - protein, 11 aa, Homo sapiens
SWIWKNSGNSF

SEQ ID NO: 986 - protein, 11 aa, Homo sapiens
SWIWKTSGNSF

SEQ ID NO: 987 - protein, 9 aa, Homo sapiens
SWIWNSFKV

SEQ ID NO: 988 - protein, 9 aa, Homo sapiens
SWKVKNLQL

SEQ ID NO: 989 - protein, 9 aa, Homo sapiens
SYKLKGNNY

SEQ ID NO: 990 - protein, 9 aa, Homo sapiens
SYKMSKDKL

SEQ ID NO: 991 - protein, 9 aa, Homo sapiens
SYKMSKTSL

SEQ ID NO: 992 - protein, 9 aa, Homo sapiens
TAAVPISCK

SEQ ID NO: 993 - protein, 10 aa, Homo sapiens
TAAVPISCKK

SEQ ID NO: 994 - protein, 10 aa, Homo sapiens
TAAVPISCKR

SEQ ID NO: 995 - protein, 8 aa, Homo sapiens
TAKRSCNL

SEQ ID NO: 996 - protein, 9 aa, Homo sapiens
TAVAVTFTK

SEQ ID NO: 997 - protein, 9 aa, Homo sapiens
TDIVKHKSV

SEQ ID NO: 998 - protein, 10 aa, Homo sapiens
TDPLDSPLRV

SEQ ID NO: 999 - protein, 11 aa, Homo sapiens
TDSENTASHGF

SEQ ID NO: 1000 - protein, 11 aa, Homo sapiens
TDSENTNQRGF

SEQ ID NO: 1001 - protein, 9 aa, Homo sapiens
TDSFSYEAL

SEQ ID NO: 1002 - protein, 8 aa, Homo sapiens
TEAASHGF

SEQ ID NO: 1003 - protein, 10 aa, Homo sapiens
TEDVPWITLI

SEQ ID NO: 1004 - protein, 9 aa, Homo sapiens
TEHCVNSEM

SEQ ID NO: 1005 - protein, 11 aa, Homo sapiens
TEKKVDLNAEW

SEQ ID NO: 1006 - protein, 10 aa, Homo sapiens
TEPNTVNITA

SEQ ID NO: 1007 - protein, 9 aa, Homo sapiens
TEPQSQLVL

SEQ ID NO: 1008 - protein, 10 aa, Homo sapiens
TEPQSQLVLA

SEQ ID NO: 1009 - protein, 9 aa, Homo sapiens
TEQASVNTV

SEQ ID NO: 1010 - protein, 11 aa, Homo sapiens
TEQASVNTVSA

SEQ ID NO: 1011 - protein, 8 aa, Homo sapiens
TESKSIFL

SEQ ID NO: 1012 - protein, 9 aa, Homo sapiens
TESQNIKKY

SEQ ID NO: 1013 - protein, 10 aa, Homo sapiens
TESQNIKKYL

SEQ ID NO: 1014 - protein, 11 aa, Homo sapiens
TESQNIKKYLF

SEQ ID NO: 1015 - protein, 11 aa, Homo sapiens
TFLRRPNFTRR

SEQ ID NO: 1016 - protein, 9 aa, Homo sapiens
TFNASFQVI

SEQ ID NO: 1017 - protein, 10 aa, Homo sapiens
TGEFIISNQV

SEQ ID NO: 1018 - protein, 9 aa, Homo sapiens
THIVYHHFF

SEQ ID NO: 1019 - protein, 8 aa, Homo sapiens
THLAQGYL

SEQ ID NO: 1020 - protein, 9 aa, Homo sapiens
THLAQGYLR

SEQ ID NO: 1021 - protein, 10 aa, Homo sapiens
TIKEAASHGF

SEQ ID NO: 1022 - protein, 9 aa, Homo sapiens
TIMNIPMEK

SEQ ID NO: 1023 - protein, 9 aa, Homo sapiens
TIMNLMNNY

SEQ ID NO: 1024 - protein, 9 aa, Homo sapiens
TKLDSLLQI

SEQ ID NO: 1025 - protein, 9 aa, Homo sapiens
TKLNVLLKL

SEQ ID NO: 1026 - protein, 9 aa, Homo sapiens
TKRAASHGF

SEQ ID NO: 1027 - protein, 9 aa, Homo sapiens
TLHPAKIKM

SEQ ID NO: 1028 - protein, 9 aa, Homo sapiens
TLIAAFRKL

SEQ ID NO: 1029 - protein, 10 aa, Homo sapiens
TLPFISLKAA

SEQ ID NO: 1030 - protein, 10 aa, Homo sapiens
TLRNIQCHLK

SEQ ID NO: 1031 - protein, 9 aa, Homo sapiens
TNILLAASR

SEQ ID NO: 1032 - protein, 10 aa, Homo sapiens
TNQRTASHGF

SEQ ID NO: 1033 - protein, 9 aa, Homo sapiens
TPIACDQNI

SEQ ID NO: 1034 - protein, 9 aa, Homo sapiens
TPIAEKDLL

SEQ ID NO: 1035 - protein, 8 aa, Homo sapiens
TPIAYFFM

SEQ ID NO: 1036 - protein, 8 aa, Homo sapiens
TQFRKLHI

SEQ ID NO: 1037 - protein, 10 aa, Homo sapiens
TQIKEDLSAF

SEQ ID NO: 1038 - protein, 10 aa, Homo sapiens
TQIKEDLSEV

SEQ ID NO: 1039 - protein, 9 aa, Homo sapiens
TQIKEKLQV

SEQ ID NO: 1040 - protein, 10 aa, Homo sapiens
TQREAASHGF

SEQ ID NO: 1041 - protein, 9 aa, Homo sapiens
TRDRDSSNL

SEQ ID NO: 1042 - protein, 9 aa, Homo sapiens
TREILKMKI

SEQ ID NO: 1043 - protein, 10 aa, Homo sapiens
TRRLQFLYRL

SEQ ID NO: 1044 - protein, 9 aa, Homo sapiens
TSFSKVRQV

SEQ ID NO: 1045 - protein, 10 aa, Homo sapiens
TSFSKVRQVF

SEQ ID NO: 1046 - protein, 9 aa, Homo sapiens
TSSDVELTK

SEQ ID NO: 1047 - protein, 10 aa, Homo sapiens
TSSGLYIFRK

SEQ ID NO: 1048 - protein, 9 aa, Homo sapiens
TSSKVELTK

SEQ ID NO: 1049 - protein, 10 aa, Homo sapiens
TSTLPCISLK

SEQ ID NO: 1050 - protein, 9 aa, Homo sapiens
TSTLPFISL

SEQ ID NO: 1051 - protein, 10 aa, Homo sapiens
TSTLPFISLK

SEQ ID NO: 1052 - protein, 10 aa, Homo sapiens
TTAVAVTFTK

SEQ ID NO: 1053 - protein, 9 aa, Homo sapiens
TTDSGSSQH

SEQ ID NO: 1054 - protein, 10 aa, Homo sapiens
TTVGIRNQSV

SEQ ID NO: 1055 - protein, 9 aa, Homo sapiens
TTVPLQTIR

SEQ ID NO: 1056 - protein, 9 aa, Homo sapiens
TVGIRNQSV

SEQ ID NO: 1057 - protein, 9 aa, Homo sapiens
TVKTQIKEK

SEQ ID NO: 1058 - protein, 8 aa, Homo sapiens
TYHLTSAL

SEQ ID NO: 1059 - protein, 8 aa, Homo sapiens
TYSIGFSY

SEQ ID NO: 1060 - protein, 9 aa, Homo sapiens
TYSIVKIAK

SEQ ID NO: 1061 - protein, 9 aa, Homo sapiens
VAFMEDDEL

SEQ ID NO: 1062 - protein, 9 aa, Homo sapiens
VANQKPFEK

SEQ ID NO: 1063 - protein, 9 aa, Homo sapiens
VANQKPLRV

SEQ ID NO: 1064 - protein, 9 aa, Homo sapiens
VAQSPRKLY

SEQ ID NO: 1065 - protein, 10 aa, Homo sapiens
VAQSPRKLYR

SEQ ID NO: 1066 - protein, 8 aa, Homo sapiens
VARIHKVF

SEQ ID NO: 1067 - protein, 9 aa, Homo sapiens
VARIHKVFA

SEQ ID NO: 1068 - protein, 9 aa, Homo sapiens
VASSFQVSK

SEQ ID NO: 1069 - protein, 10 aa, Homo sapiens
VDLNADPLCY

SEQ ID NO: 1070 - protein, 8 aa, Homo sapiens
VEELVEAL

SEQ ID NO: 1071 - protein, 9 aa, Homo sapiens
VEKASLPNL

SEQ ID NO: 1072 - protein, 10 aa, Homo sapiens
VEVTKSPRKL

SEQ ID NO: 1073 - protein, 11 aa, Homo sapiens
VEVTKSPRKLY

SEQ ID NO: 1074 - protein, 9 aa, Homo sapiens
VGFRGSWHK

SEQ ID NO: 1075 - protein, 9 aa, Homo sapiens
VLLFYNVHM

SEQ ID NO: 1076 - protein, 9 aa, Homo sapiens
VLLKQKAVK

SEQ ID NO: 1077 - protein, 9 aa, Homo sapiens
VLNCKLIVV

SEQ ID NO: 1078 - protein, 10 aa, Homo sapiens
VLSNLVIQNV

SEQ ID NO: 1079 - protein, 9 aa, Homo sapiens
VLSNLVMKR

SEQ ID NO: 1080 - protein, 9 aa, Homo sapiens
VLVKVLLFY

SEQ ID NO: 1081 - protein, 10 aa, Homo sapiens
VMISRGIQNV

SEQ ID NO: 1082 - protein, 10 aa, Homo sapiens
VMISRIIQNV

SEQ ID NO: 1083 - protein, 9 aa, Homo sapiens
VMKRIIQNV

SEQ ID NO: 1084 - protein, 10 aa, Homo sapiens
VPSLACELTL

SEQ ID NO: 1085 - protein, 10 aa, Homo sapiens
VPVKTQIKEA

SEQ ID NO: 1086 - protein, 9 aa, Homo sapiens
VPVNGLLEL

SEQ ID NO: 1087 - protein, 11 aa, Homo sapiens
VPVNGLLELTL

SEQ ID NO: 1088 - protein, 8 aa, Homo sapiens
VPWITLIA

SEQ ID NO: 1089 - protein, 9 aa, Homo sapiens
VPWITLIAA

SEQ ID NO: 1090 - protein, 9 aa, Homo sapiens
VPWITLNEV

SEQ ID NO: 1091 - protein, 8 aa, Homo sapiens
VQHSLPTL

SEQ ID NO: 1092 - protein, 10 aa, Homo sapiens
VQIPVNLKNV

SEQ ID NO: 1093 - protein, 9 aa, Homo sapiens
VRKISQLIM

SEQ ID NO: 1094 - protein, 10 aa, Homo sapiens
VSKEALGFVY

SEQ ID NO: 1095 - protein, 9 aa, Homo sapiens
VSRNQAHLI

SEQ ID NO: 1096 - protein, 10 aa, Homo sapiens
VTDSENTHGF

SEQ ID NO: 1097 - protein, 9 aa, Homo sapiens
VTDSENTIK

SEQ ID NO: 1098 - protein, 9 aa, Homo sapiens
VTDSENTNH

SEQ ID NO: 1099 - protein, 11 aa, Homo sapiens
VTDSENTNHGF

SEQ ID NO: 1100 - protein, 9 aa, Homo sapiens
VTDSENTNK

SEQ ID NO: 1101 - protein, 9 aa, Homo sapiens
VTEAASHGF

SEQ ID NO: 1102 - protein, 10 aa, Homo sapiens
VTIMNIPMEK

SEQ ID NO: 1103 - protein, 10 aa, Homo sapiens
VTIMNLMNNY

SEQ ID NO: 1104 - protein, 9 aa, Homo sapiens
VTKSPRKLY

SEQ ID NO: 1105 - protein, 10 aa, Homo sapiens
VTKSPRKLYR

SEQ ID NO: 1106 - protein, 9 aa, Homo sapiens
VTLHPAKIK

SEQ ID NO: 1107 - protein, 9 aa, Homo sapiens
VTVKHTNQR

SEQ ID NO: 1108 - protein, 9 aa, Homo sapiens
VTVKTQNQR

SEQ ID NO: 1109 - protein, 9 aa, Homo sapiens
VVPSLACEL

SEQ ID NO: 1110 - protein, 10 aa, Homo sapiens
VVPVNGLLEL

SEQ ID NO: 1111 - protein, 9 aa, Homo sapiens
VVSKEALGF

SEQ ID NO: 1112 - protein, 10 aa, Homo sapiens
WAQMEKIPLL

SEQ ID NO: 1113 - protein, 9 aa, Homo sapiens
WIDHIGKSM

SEQ ID NO: 1114 - protein, 9 aa, Homo sapiens
WITLIAAFR

SEQ ID NO: 1115 - protein, 10 aa, Homo sapiens
WITLNEVDEY

SEQ ID NO: 1116 - protein, 9 aa, Homo sapiens
WIWKNIREF

SEQ ID NO: 1117 - protein, 9 aa, Homo sapiens
WIWKNIREV

SEQ ID NO: 1118 - protein, 9 aa, Homo sapiens
WIWKNIREY

SEQ ID NO: 1119 - protein, 9 aa, Homo sapiens
WIWKNISKV

SEQ ID NO: 1120 - protein, 9 aa, Homo sapiens
WKALEDFTL

SEQ ID NO: 1121 - protein, 9 aa, Homo sapiens
WLVRKISQL

SEQ ID NO: 1122 - protein, 10 aa, Homo sapiens
WPNLPDSQQV

SEQ ID NO: 1123 - protein, 9 aa, Homo sapiens
WPVNGLLEL

SEQ ID NO: 1124 - protein, 11 aa, Homo sapiens
WPVNGLLELTL

SEQ ID NO: 1125 - protein, 9 aa, Homo sapiens
WSPDGKIPL

SEQ ID NO: 1126 - protein, 10 aa, Homo sapiens
WSPDGKIPLL

SEQ ID NO: 1127 - protein, 10 aa, Homo sapiens
WSPMEKIPLL

SEQ ID NO: 1128 - protein, 10 aa, Homo sapiens
WSQMEKIPLL

SEQ ID NO: 1129 - protein, 10 aa, Homo sapiens
YEETDITESV

SEQ ID NO: 1130 - protein, 8 aa, Homo sapiens
YENNSNYY

SEQ ID NO: 1131 - protein, 9 aa, Homo sapiens
YENNSNYYS

SEQ ID NO: 1132 - protein, 9 aa, Homo sapiens
YFETEAVAF

SEQ ID NO: 1133 - protein, 10 aa, Homo sapiens
YFETEAVAFM

SEQ ID NO: 1134 - protein, 9 aa, Homo sapiens
YHHFFPSSH

SEQ ID NO: 1135 - protein, 10 aa, Homo sapiens
YHHFFPSSHL

SEQ ID NO: 1136 - protein, 11 aa, Homo sapiens
YHHFFPSSHLL

SEQ ID NO: 1137 - protein, 10 aa, Homo sapiens
YHLSSEATKL

SEQ ID NO: 1138 - protein, 9 aa, Homo sapiens
YHLTSALKV

SEQ ID NO: 1139 - protein, 9 aa, Homo sapiens
YIFRNERKK

SEQ ID NO: 1140 - protein, 9 aa, Homo sapiens
YKHPGSFLI

SEQ ID NO: 1141 - protein, 8 aa, Homo sapiens
YKMSKTSL

SEQ ID NO: 1142 - protein, 8 aa, Homo sapiens
YLRGAKKL

SEQ ID NO: 1143 - protein, 9 aa, Homo sapiens
YLSKVFSKV

SEQ ID NO: 1144 - protein, 9 aa, Homo sapiens
YLYENNSNY

SEQ ID NO: 1145 - protein, 10 aa, Homo sapiens
YLYENNSNYY

SEQ ID NO: 1146 - protein, 8 aa, Homo sapiens
YLYETTIA

SEQ ID NO: 1147 - protein, 9 aa, Homo sapiens
YLYETTIAE

SEQ ID NO: 1148 - protein, 10 aa, Homo sapiens
YRKEWNKQKL

SEQ ID NO: 1149 - protein, 9 aa, Homo sapiens
YRLWPVNGL

SEQ ID NO: 1150 - protein, 10 aa, Homo sapiens
YRLWPVNGLL

SEQ ID NO: 1151 - protein, 9 aa, Homo sapiens
YRQLISLQA

SEQ ID NO: 1152 - protein, 10 aa, Homo sapiens
YRQLISLQAF

SEQ ID NO: 1153 - protein, 9 aa, Homo sapiens
YSAHGTEAL

SEQ ID NO: 1154 - protein, 9 aa, Homo sapiens
YSKDSENAK

SEQ ID NO: 1155 - protein, 9 aa, Homo sapiens
YSVVNSSAF

SEQ ID NO: 1156 - protein, 10 aa, Homo sapiens
YTNQSPYSVV

SEQ ID NO: 1157 - protein, 12 aa, Homo sapiens
SHLSGVDQGTCL

SEQ ID NO: 1158 - protein, 9 aa, Homo sapiens
NAMQKILVS

SEQ ID NO: 1159 - protein, 11 aa, Homo sapiens
NAMQKILVSHL

SEQ ID NO: 1160 - protein, 9 aa, Homo sapiens
AMQKILVSH

SEQ ID NO: 1161 - protein, 10 aa, Homo sapiens
AMQKILVSHL

SEQ ID NO: 1162 - protein, 8 aa, Homo sapiens
MQKILVSH

SEQ ID NO: 1163 - protein, 11 aa, Homo sapiens
LSGVDQGTCLH

SEQ ID NO: 1164 - protein, 10 aa, Homo sapiens
SGVDQGTCLH

SEQ ID NO: 1165 - protein, 9 aa, Homo sapiens
GVDQGTCLH

SEQ ID NO: 1166 - protein, 10 aa, Homo sapiens
ARESPGQKDL

SEQ ID NO: 1167 - protein, 9 aa, Homo sapiens
GPKRARESP

SEQ ID NO: 1168 - protein, 11 aa, Homo sapiens
RARESPGQKDL

SEQ ID NO: 1169 - protein, 11 aa, Homo sapiens
GQKDLQGARNL

SEQ ID NO: 1170 - protein, 12 aa, Homo sapiens
KDLQGARNLLLW

SEQ ID NO: 1171 - protein, 9 aa, Homo sapiens
DLQGARNLL

SEQ ID NO: 1172 - protein, 10 aa, Homo sapiens
LQGARNLLLW

SEQ ID NO: 1173 - protein, 11 aa, Homo sapiens
QGARNLLLWAL

SEQ ID NO: 1174 - protein, 10 aa, Homo sapiens
ARNLLLWALH

SEQ ID NO: 1175 - protein, 12 aa, Homo sapiens
ARNLLLWALHQH

SEQ ID NO: 1176 - protein, 9 aa, Homo sapiens
LLWALHQHA

SEQ ID NO: 1177 - protein, 10 aa, Homo sapiens
HQHAHRSTGM

SEQ ID NO: 1178 - protein, 12 aa, Homo sapiens
HRSTGMDGTAVW

SEQ ID NO: 1179 - protein, 12 aa, Homo sapiens
RSTGMDGTAVWC

SEQ ID NO: 1180 - protein, 11 aa, Homo sapiens
STGMDGTAVWC

SEQ ID NO: 1181 - protein, 8 aa, Homo sapiens
GAFIIHPW

SEQ ID NO: 1182 - protein, 12 aa, Homo sapiens
HRCPPNCGCAAR

SEQ ID NO: 1183 - protein, 11 aa, Homo sapiens
CAARCLDRGQW

SEQ ID NO: 1184 - protein, 9 aa, Homo sapiens
ARCLDRGQW

SEQ ID NO: 1185 - protein, 8 aa, Homo sapiens
RCLDRGQW

SEQ ID NO: 1186 - protein, 9 aa, Homo sapiens
RGQWLPCNW

SEQ ID NO: 1187 - protein, 11 aa, Homo sapiens
KSMILKSEEMW

SEQ ID NO: 1188 - protein, 9 aa, Homo sapiens
SMILKSEEM

SEQ ID NO: 1189 - protein, 10 aa, Homo sapiens
SMILKSEEMW

SEQ ID NO: 1190 - protein, 9 aa, Homo sapiens
MILKSEEMW

SEQ ID NO: 1191 - protein, 9 aa, Homo sapiens
QENPQDRKI

SEQ ID NO: 1192 - protein, 8 aa, Homo sapiens
MILKSEEM

SEQ ID NO: 1193 - protein, 8 aa, Homo sapiens
ILKSEEMW

SEQ ID NO: 1194 - protein, 10 aa, Homo sapiens
ILKSEEMWSM

SEQ ID NO: 1195 - protein, 9 aa, Homo sapiens
LKSEEMWSM

SEQ ID NO: 1196 - protein, 11 aa, Homo sapiens
EMWSMEETTKV

SEQ ID NO: 1197 - protein, 9 aa, Homo sapiens
WSMEETTKV

SEQ ID NO: 1198 - protein, 11 aa, Homo sapiens
SEQENPQDRKI

SEQ ID NO: 1199 - protein, 12 aa, Homo sapiens
SEQENPQDRKIF

SEQ ID NO: 1200 - protein, 10 aa, Homo sapiens
QENPQDRKIF

SEQ ID NO: 1201 - protein, 8 aa, Homo sapiens
NPQDRKIF

SEQ ID NO: 1202 - protein, 11 aa, Homo sapiens
NPQDRKIFRGL

SEQ ID NO: 1203 - protein, 11 aa, Homo sapiens
FSTARENLSRY

SEQ ID NO: 1204 - protein, 10 aa, Homo sapiens
LHYKTQDKCF

SEQ ID NO: 1205 - protein, 11 aa, Homo sapiens
LHYKTQDKCFL

SEQ ID NO: 1206 - protein, 11 aa, Homo sapiens
STARENLSRYQ

SEQ ID NO: 1207 - protein, 11 aa, Homo sapiens
ARENLSRYQML

SEQ ID NO: 1208 - protein, 8 aa, Homo sapiens
ENLSRYQM

SEQ ID NO: 1209 - protein, 11 aa, Homo sapiens
NLSRYQMLHYK

SEQ ID NO: 1210 - protein, 10 aa, Homo sapiens
LSRYQMLHYK

SEQ ID NO: 1211 - protein, 10 aa, Homo sapiens
SRYQMLHYKT

SEQ ID NO: 1212 - protein, 11 aa, Homo sapiens
SRYQMLHYKTQ

SEQ ID NO: 1213 - protein, 10 aa, Homo sapiens
QMLHYKTQDK

SEQ ID NO: 1214 - protein, 9 aa, Homo sapiens
MLHYKTQDK

SEQ ID NO: 1215 - protein, 9 aa, Homo sapiens
LHYKTQDKC

SEQ ID NO: 1216 - protein, 9 aa, Homo sapiens
HYKTQDKCF

SEQ ID NO: 1217 - protein, 9 aa, Homo sapiens
YKTQDKCFL

SEQ ID NO: 1218 - protein, 9 aa, Homo sapiens
HSKGKSVQV

SEQ ID NO: 1219 - DNA, 15 bp, Homo sapiens
  source 1..15: mol_type = genomic DNA
tagagtgtcccatct

SEQ ID NO: 1220 - DNA, 11 bp, Homo sapiens
  source 1..11: mol_type = genomic DNA
ttttctgaaat

SEQ ID NO: 1221 - DNA, 16 bp, Homo sapiens
  source 1..16: mol_type = genomic DNA
gcaagtggaaaatctg

SEQ ID NO: 1222 - DNA, 14 bp, Homo sapiens
  source 1..14: mol_type = genomic DNA
caagtgttttctga

SEQ ID NO: 1223 - protein, 26 aa, Homo sapiens
NVINAMQKILVSHLSGVDQGTCLHKV

SEQ ID NO: 1224 - protein, 36 aa, Homo sapiens
KSMILKSEEMWSMEETTKVQSEQENPQDRKIFRGLE

SEQ ID NO: 1225 - protein, 83 aa, Homo sapiens
HQGPKRARESPGQKDLQGARNLLLWALHQHAHRSTGMDGTAVWCFCGEGAFIIHPWHRCPPNCGCAARCLDRGQWLPCNWADV

SEQ ID NO: 1226 - protein, 13 aa, Homo sapiens
HSKGKSVQVSDAS

SEQ ID NO: 1227 - protein, 31 aa, Homo sapiens
ANTCGIFSTARENLSRYQMLHYKTQDKCFLK

SEQ ID NO: 1228 - DNA, 45 bp, Homo sapiens
  source 1..45: mol_type = genomic DNA
cctgcagaagaatctgaacataaaaacaacaattacgaaccaaac

SEQ ID NO: 1229 - DNA, 45 bp, Homo sapiens
  source 1..45: mol_type = genomic DNA
cctgcagaataatctgaacataaaaacaacaattacgaaccaaac

SEQ ID NO: 1230 - DNA, 21 bp, Homo sapiens
  source 1..21: mol_type = genomic DNA
cctgcagaatacgaaccaaac

SEQ ID NO: 1231 - protein, 55 aa, Homo sapiens
FSTASGKSVQVSDASLQNARQVFSEIEDSTKQVFSKVLFKSNEHSDQLTREENTA

SEQ ID NO: 1232 - protein, 26 aa, Homo sapiens
  VARIANT 26: note = X can be any naturally occurring amino acid
FSTARENLSRYQMLHYKTQDKCFLKX

SEQ ID NO: 1233 - protein, 52 aa, Homo sapiens
FSTARENLSRYQMLHYKTQDKCKEDSTKQVFSKVLFKSNEHSDQLTREENTA

SEQ ID NO: 1234 - protein, 54 aa, Homo sapiens
FSTARENLSRYQMLHYKTQDISSEIEDSTKQVFSKVLFKSNEHSDQLTREENTA

SEQ ID NO: 1235 - protein, 50 aa, Homo sapiens
FSTARENLSRYQMLHYKTQEIEDSTKQVFSKVLFKSNEHSDQLTREENTA

SEQ ID NO: 1236 - protein, 43 aa, Homo sapiens
FSTARENLSRFSEIEDSTKQVFSKVLFKSNEHSDQLTREENTA

SEQ ID NO: 1237 - protein, 52 aa, Homo sapiens
FSTARENLSDASLQNARQVFSEIEDSTKQVFSKVLFKSNEHSDQLTREENTA

SEQ ID NO: 1238 - protein, 9 aa, Homo sapiens
FSTARENTA

SEQ ID NO: 1239 - protein, 52 aa, Homo sapiens
FSTAREKVSDASLQNARQVFSEIEDSTKQVFSKVLFKSNEHSDQLTREENTA

SEQ ID NO: 1240 - protein, 56 aa, Homo sapiens
FSTAREKKSVQVSDASLQNARQVFSEIEDSTKQVFSKVLFKSNEHSDQLTREENTA

SEQ ID NO: 1241 - protein, 40 aa, Homo sapiens
FSTARQVFSEIEDSTKQVFSKVLFKSNEHSDQLTREENTA

SEQ ID NO: 1242 - protein, 52 aa, Homo sapiens
FSTASVQVSDASLQNARQVFSEIEDSTKQVFSKVLFKSNEHSDQLTREENTA

SEQ ID NO: 1243 - protein, 4 aa, Homo sapiens
RENL

SEQ ID NO: 1244 - protein, 5 aa, Homo sapiens
RENLS

SEQ ID NO: 1245 - protein, 6 aa, Homo sapiens
RENLSR

SEQ ID NO: 1246 - protein, 7 aa, Homo sapiens
RENLSRY

SEQ ID NO: 1247 - protein, 15 aa, Homo sapiens
RENLSRYQMLHYKTQ

(19) United States
(12) Patent Application Publication
(10) Pub. No.: US 2026/0286395 A1
(43) Pub. Date: Sep. 24, 2026

(54) ANTI-CANCER VACCINES AND RELATED THERAPY

(71) Applicant: The Institute of Cancer Research: Royal Cancer Hospital, London (GB)

(72) Inventors: Stephen Pettitt, London (GB); Christopher Lord, London (GB); Marco Punta, London (GB); Alan Melcher, London (GB)

(73) Assignee: The Institute of Cancer Research: Royal Cancer Hospital, London (GB)

(21) Appl. No.: 19/702,550

(22) Filed: Jun. 9, 2026

Related U.S. Application Data
(62) Division of application No. 17/921,837, filed on Apr. 28, 2021, now Pat. No. 12,674,179.

(30) Foreign Application Priority Data
Apr. 28, 2020 (GB) 2006254.3

(51) Int. Cl. C12N 15/86; A61K 35/17; A61K 40/11; A61K 40/31; A61K 40/42; A61P 35/00; C12N 15/90

(52) U.S. Cl. CPC C12N 15/86; A61K 35/17; A61K 40/11; A61K 40/31; A61K 40/4201; A61P 35/00; C12N 15/902

(57) ABSTRACT

The present invention provides an anti-cancer vaccine comprising: (i) at least one peptide comprising the amino acid sequence of a neoantigen encoded by a mutant homologous recombination (HR) DNA repair gene selected from the group: BRCA1, BRCA2, PALB2, CDK12, RAD51B, RAD51C and RAD51D, wherein the mutant gene comprises a reversion mutation; and/or (ii) at least one polynucleotide encoding the at least one peptide of (i). Also provided are engineered T cells that recognise said neoantigen. Related methods and medical uses of the vaccine and/or engineered T cell are provided, including for the treatment of cancers, such as homologous recombination (HR) deficient cancers that acquire PARP inhibitor resistance or platinum resistance by development of reversion mutations in an HR DNA repair gene selected from the group: BRCA1, BRCA2, PALB2, CDK12, RAD51B, RAD51C and RAD51D.

### CROSS REFERENCE TO RELATED APPLICATIONS

This application is a divisional of U.S. patent application Ser. No. 17/921,837, filed Oct. 27, 2022, which is the § 371 U.S. National Stage of International Application No. PCT/EP2021/061184, filed Apr. 28, 2021, which was published in English under PCT Article 21 (2), which in turn which claims priority from GB2006254.3 filed 28 Apr. 2020, the contents and elements of which are herein incorporated by reference for all purposes.

### SEQUENCE LISTING INCORPORATION

This application contains a sequence listing as part of the description. The sequences set forth in the sequence listing form part of the present description just as if each of the amino acid sequences and nucleotide sequences of the sequence listing had been individually set forth in the main body of the description. The Sequence Listing is submitted as an XML file in the form of the file named 6947-109208-02_ST26.xml, which was created on Jun. 8, 2026, and is 1,073, 737 bytes, which is incorporated by reference herein.

### FIELD OF THE INVENTION

The present invention relates to products and methods for the treatment of certain cancers. In particular, vaccine-based therapy for the prevention or treatment of drug resistant cancers is disclosed.

### BACKGROUND TO THE INVENTION

Defects in genes that control homologous recombination (HR) DNA repair, such as BRCA1, BRCA2, RAD51C, RAD51D and PALB2, are common in cancer and are enriched in high grade serous ovarian cancers (HGSOC (Cancer Genome Atlas Research, 2011)), triple-negative breast cancer (TNBC (Cancer Genome Atlas, 2012; Staaf et al., 2019)) castrate resistant metastatic prostate cancer (Grasso et al., 2012) and pancreatic cancer (Bailey et al., 2016; Holter et al., 2015; Waddell et al., 2015). Following the pre-clinical identification of synthetic lethality between BRCA1/2-mutation and poly ADP ribose polymerase (PARP) PARP inhibitors (PARPi) (Lord and Ashworth, 2016, 2017), a number of clinical trials demonstrated that PARPi, as well as platinum, are effective in patients with either germ-line or somatic HR gene mutations, leading to the approval of four different PARPi for the treatment of HR-defective breast or ovarian cancers, and the increased use of platinum in a similar clinical context (Alsop et al., 2012; Lord and Ashworth, 2017; Tutt, 2018; Tutt et al., 2018).

Platinum salts and PARPi are now widely used to treat cancers with mutations in HR genes, including BRCA1 and BRCA2 (Alsop et al. 2012; Lord and Ashworth 2017; Tutt et al. 2018). Resistance to these agents frequently emerges, especially in the advanced disease setting and, in cases where the original pathogenic BRCA1/2 mutation causes a frameshift, is often via secondary, or reversion, mutations that restore the native reading frame of the mutated gene (Edwards et al. 2008; Sakai et al. 2008; Lin et al. 2019).

There remains an unmet need for therapies that prevent or treat drug resistant cancers, including the growing population of PARPi or platinum salt resistant cancers caused by reversion mutations. The present invention addresses these and other needs, and provides related advantages as described herein.

### BRIEF DESCRIPTION OF THE INVENTION

Broadly, the present invention relates to cancer treatment. The present inventors have surprisingly found that homologous recombination (HR) deficient cancers that acquire PARP inhibitor resistance or platinum resistance by development of reversion mutations in genes that encode (HR) DNA repair proteins, such as BRCA1, BRCA2, PALB2, CDK12, RAD51B, RAD51C and RAD51D, frequently encode neoantigen sequence by virtue of regions of out-of-frame amino acid sequence. MHC presentation predictor tools indicate that the neoantigen sequence is capable of eliciting an immune response that opens the door to novel and effective immunotherapy for the cancer. In particular, anti-cancer vaccines based on the neoantigen encoded by the reversion mutation (or encoded by the primary mutation and stabilised by the reversion mutation) and/or engineered T cells that recognise the neoantigen may be used to treat the cancer, including by preventing or reversing acquisition of PARP inhibitor resistance or platinum resistance.

Accordingly, in a first aspect of the invention, there is provided an anti-cancer vaccine comprising: (i) at least one peptide comprising the amino acid sequence of a neoantigen encoded by a mutant HR DNA repair gene selected from the group: BRCA1, BRCA2, PALB2, CDK12, RAD51B, RAD51C and RAD51D, wherein said mutant gene comprises a reversion mutation; and/or (ii) at least one polynucleotide encoding the at least one peptide of (i).

In some embodiments the neoantigen is encoded by a portion of the mutant gene comprising the reversion mutation.

In some embodiments the neoantigen is encoded by a portion of the mutant gene comprising a primary mutation (e.g. a truncation mutation). Expression of the neoantigen sequence may be stabilised by the presence of a reversion mutation in the gene. Neoantigen sequence encoded by primary mutations that are stabilised by a reversion mutation are considered particularly attractive targets for vaccine use because such neoantigen sequences are more likely to reoccur between patients with the same or similar primary mutations, thereby offering potentially greater therapeutic utility across a range of patients (e.g. patients having the same primary mutation, but different reversion mutations).

In some embodiments the neoantigen may be associated with a common founder mutation and comprise a peptide of 8-12 amino acids derived from the following sequences, including at least some of the underlined sequence (the out-of-frame sequence) and potentially including some additional flanking sequence:

BRCA1:c.185delAG [aka 68_69delAG]—these names may be used interchangeably. Downstream: NVINAMQKILVSHLSGVDQGTCLHKV (SEQ ID NO: 1223). Note that there is no upstream sequence for this mutation due to the stop codon position.

BRCA1:c.5382insC [aka 5266insC or dupC]. Upstream: KSMILKSEEMWSMEETTKVQSEQENPQDRKIFRGLE (SEQ ID NO: 1224). Downstream: HQGPKRARESPGQKDLQGARNLLLWALHQHAHRSTGMDGTAVWCFCGEGAFIIHPWHRCPPNCGC AARCLDRGQWLPCNWADV (SEQ ID NO: 1225).

BRCA2:c.6174delT [aka 5946delT]. Upstream: HSKGKSVQVSDAS (SEQ ID NO: 1226). Downstream: ANTCGIFSTARENLSRYQMLHYKTQDKCELK (SEQ ID NO: 1227).

In some embodiments the neoantigen comprises or consists of an amino acid sequence set forth in any one of SEQ ID Nos: 1-1218, 1223-1227, and/or 1231-1247.

In some embodiments the neoantigen comprises or consists of an amino acid sequence set forth in any one of SEQ ID Nos: 1-89.

In some embodiments the neoantigen comprises or consists of an amino acid sequence selected from the group consisting of: the peptide sequences set forth in Table 5 and Table 6.

In some embodiments the neoantigen comprises or consists of an amino acid sequence selected from the group consisting of the peptide sequences set forth in Table 5. These neoantigen sequence, being encoded by primary mutations (and stabilised by reversion mutations) are considered particularly attractive targets for vaccine use because such neoantigen sequences are more likely to reoccur between patients with the same or similar primary mutations, thereby offering potentially greater therapeutic utility across a range of patients (e.g. patients having the same primary mutation, but different reversion mutations). The primary mutations may be common founder mutations. However, it is also contemplated that the primary mutation may be a less common primary mutation.

Using the NetMHCpan 4.0 algorithm (Jurtz et al., 2017), the neoantigen sequences (see, e.g., those set forth in Tables 5 and 6) were predicted to be presented by the MHC in a significant proportion of individuals (in some cases at least 75% of individuals taking into account the population frequencies of different HLA types) making them particularly preferred for vaccine therapy. However, in some embodiments the vaccine may form part of a personalized medicine strategy, wherein the neoantigen sequence is determined by sequencing a mutant HR DNA repair gene selected from the group: BRCA1, BRCA2, PALB2, CDK12, RAD51B, RAD51C and RAD51D from a tumour sample, or from circulating tumour DNA, from a subject and then tailoring the vaccine to that same subject by employing a neoantigen sequence found to be present in the tumour of the subject. In such cases, it is the predicted or actual degree of MHC presentation by the HLA allotype of the subject concerned that is relevant rather than a population-level degree of MHC presentation.

In some embodiments the neoantigen comprises the amino acid sequence: RENLSRYQMLHYKTQ (SEQ ID NO: 1247). Peptides derived from this neoantigen sequence (potentially also including some of the 5′ flanking wild type BRCA2 sequence), which is encoded by patients with observed reversion mutations, were found to have a high immunogenicity as measured by best predicted rank among possible neoantigens. In particular embodiments, the neoantigen peptide may comprise, for example, 1, 2, 3, 4 or more amino acids encoded by 5′ flanking wild-type BRCA2 sequence (see FIGS. 4B and 4C) (e.g. A, TA, STA, etc. may form additional sequence N-terminal of the amino acid sequence of SEQ ID NO: 1247. Peptides derived from the neoantigen of SEQ ID NO: 1247 have strong binding affinity predicted for many HLA alleles, indicating that they are likely to be presented to the immune system. This neoantigen is encoded by out-of-frame protein sequence following the BRCA2:c.5946delT founder mutation and may be of use in treating cancers associated with this mutation.

In some embodiments the vaccine comprises a DNA or RNA sequence encoding said neoantigen. The DNA or RNA may be single-stranded or double-stranded. The RNA sequence may be mRNA. In some cases the DNA or RNA sequence is provided in the form of a vector, such as a viral vector. In particular, the vaccine may comprise an oncolytic virus. Examples of oncolytic viruses include: viruses based on herpes simplex virus-1 (HSV-1), such as Talimogene laherparepvec; an oncolytic adenovirus; and an oncolytic adeno-associated virus (AAV).

In some embodiments the vaccine is in the form of a plurality of dendritic cells (DCs) that have been pulsed with the at least one peptide comprising the neoantigen. The DCs are capable of presenting the neoantigen to one or more T cells when administered to a subject. The DCs may be cultured ex vivo, optionally expanded and/or matured prior to being contacted with the neoantigen peptide(s). Injection of the DCs may then stimulate T cells in vivo and thereby facilitate the development of an immune response against the tumour.

The neoantigen is typically an MHC class I restricted peptide. Such MHC I binding peptides are typically 8-13 amino acids in length. However, MHC class II restricted peptides (typically longer, such as 15-24 amino acids in length) are also specifically contemplated herein.

The neoantigen may have a sequence that is predicted to be presented by at least 10%, 25%, 50% or at least 75% of the HLA-A, HLA-B and/or HLA-C allotypes of the 1000 Genomes dataset as disclosed by Gourraud et al., 2014, PLOS ONE, Vol. 9, e97282.

The neoantigen may have a sequence that is predicted to be presented by MHC class I by an MHC class I predictor. The skilled person will be aware of a number of algorithms that are widely used to predict whether a peptide sequence will be displayed by MHC class I. These include: NNAlign-2.0, NetMHC, NetMHCpan and NetMHCpan-4.0. Preferably the neoantigen sequence is predicted to be displayed by MHC class I by NetMHCpan-4.0 (Jurtz et al., (2017) J. Immunol., Vol. 199, pp. 3360-3368). NetMHCpan-4.0 is a neural network that was trained on binding affinity and eluted ligand data leveraging the information from both data types. Large-scale benchmarking has shown that NetMHCpan-4.0 demonstrates enhanced predicted performance compared with other methods when it comes to identification of naturally processed ligands, cancer neoantigens and T cell epitopes. In some cases the neoantigen has a sequence that exhibits a best rank (BR) score of 0.5 or less using the NetMHCpan-4.0 neural network predictor.

In some the vaccine comprises 2, 3, 4, 5, 6, 7, 8, 9, 10 or more different neoantigens and/or polynucleotide encoding the different neoantigens (for example the neoantigen sequences set forth in SEQ ID Nos: 1-1218, 1228-1247, Table 5 and Table 6). In some cases the plurality of different neoantigens may be different neoantigens that are encoded by different HR DNA repair gene mutations (e.g. a gene selected from the group: BRCA1, BRCA2, PALB2, CDK12, RAD51B, RAD51C and RAD51D, wherein said gene comprises a reversion mutation). In some cases, the plurality of different neoantigens may combine at least one neoantigen that is encoded by a BRCA1, BRCA2, PALB2, CDK12, RAD51B, RAD51C or RAD51D reversion mutations together with a neoantigen encoded by a different gene. Combining two or more (such as several, e.g. 3, 4, 5, 6, 7, 8, 9, 10 or more) neoantigens in a single vaccine (“multi-epitope vaccination”) may be employed to increase the efficacy of the vaccine, including by combating the problems of epitope loss by malignant cells and/or an immune-suppressive tumour microenvironment.

In some cases the anti-cancer vaccine of the invention further comprises at least one adjuvant. In some cases the adjuvant may be a toll-like receptor (TLR) agonist, such as an agonist of TLR3 (e.g. polyinosinic-polycytidylic acid), TLR4 (monophos-phoryl lipid A), TLR7 (imiquimod), TLR8 (resiquimod) and TLR9 (CpG oligodeoxynucleotide). In some cases the adjuvant may comprise a monoclonal antibody that targets the neoantigen to DCs (e.g. anti-DEC205. In some cases the vaccine may be delivered in the form of or in conjunction with a nanoparticle, such as a nanoparticle that targets the neoantigen to antigen presenting cells.

In a second aspect the present invention provides an engineered T cell that recognises a neoantigen encoded by a mutant HR DNA repair gene selected from the group: BRCA1, BRCA2, PALB2, CDK12, RAD51B, RAD51C and RAD51D. The reversion mutation and/or neoantigen may be as defined in connection with the first aspect of the invention. In particular, the neoantigen may have an amino acid sequence set forth in any one of SEQ ID Nos: 1-1218, such as for example, any one of SEQ ID Nos: 1-89. In particular, the neoantigen may have an amino acid sequence as set forth for a peptide of Table 5 or Table 6. In some cases the engineered T cell is selected from: a chimeric antigen receptor T cell (CAR-T), an engineered T cell receptor (TCR) T cell and a neoantigen-reactive T cell (NAR-T).

In a third aspect the present invention provides the anti-cancer vaccine of the first aspect of the invention or the engineered T cell of the second aspect of the invention for use in medicine.

In a fourth aspect the present invention provides the anti-cancer vaccine of the first aspect of the invention or the engineered T cell of the second aspect of the invention for use in a method of treatment of a proliferative disorder in a mammalian subject.

In some embodiments the proliferative disorder is a cancer that exhibits a homologous recombination (HR) defect. In particular, a mutation in one or more of BRCA1, BRCA2, CDK12, RAD51B, RAD51C, RAD51D and PALB2. In some cases, the mutation may be a truncation mutation.

In some embodiments the proliferative disorder is a cancer, such as a solid tumour. The proliferative disorder may, in some embodiments, be selected from: high grade serous ovarian cancer (HGSOC), triple-negative breast cancer (TNBC), castrate resistant metastatic prostate cancer and pancreatic cancer.

In some embodiments the method of treatment comprises the inhibition or prevention of development of, or reduction or reversal of tumour resistance to PARP inhibitor therapy and/or platinum therapy.

In some embodiments the subject has a mutation in the BRCA1 gene, BRCA2 gene, PALB2 gene, CDK12 gene, RAD51B gene, RAD51C gene and/or RAD51D gene. In particular, the mutation may be a somatic mutation. A subject having a germ line mutation (usually heterozygous) in the BRCA1 gene, BRCA2 gene, PALB2 gene, CDK12 gene, RAD51B gene, RAD51C gene and/or RAD51D gene, may potentially have central immune tolerance to the out-of-frame sequence associated with that mutation. This sequence would theoretically be shared with the primary tumor (e.g. in cases of somatic BRCA mutation) or with heterozygous normal cells in the body in carriers of BRCA germ line mutations, and thus may have previously been exposed to the immune system during development of central tolerance. Additionally, it is possible that stimulation of an immune response against reversions using a vaccine might result in development of auto-immunity in BRCA carriers in cases where the neoantigen sequence is shared between reversion and pathogenic mutations. For these reasons, in some embodiments the subject may have a somatic mutation in the BRCA1 gene, BRCA2 gene, PALB2 gene, CDK12 gene, RAD51B gene, RAD51C gene and/or RAD51D gene without having that mutation in his or her germ line (i.e. a non-carrier). In other words, a non-carrier subject may in some cases be preferred for vaccine therapy of the present invention owing to the non-carrier subject being less likely to have central tolerance for the out-of-frame sequence and/or less likely to develop possible autoimmune complications.

In some embodiments the BRCA1 mutation, BRCA2 mutation, PALB2 mutation, CDK12 mutation, RAD51B mutation, RAD51C mutation and/or RAD51D mutation is a reversion mutation, optionally wherein the reversion mutation is as set forth in Table 5 or Table 6.

In some embodiments the method of treatment comprises a step of determining whether said BRCA1 mutation, said BRCA2 mutation, said PALB2 mutation, said CDK12 mutation, said RAD51B mutation, RAD51C mutation and/or said RAD51D mutation is present in a tumour of the subject. This may involve analysis (e.g. sequencing) of a DNA or RNA containing sample (e.g. a ctDNA sample) obtained from the subject or if a tumour sample (e.g. biopsy sample) obtained from the subject.

In some embodiments the tumour is determined have a BRCA1 mutation, BRCA2 mutation, PALB2 mutation, CDK12 mutation, RAD51B mutation, RAD51C mutation and/or RAD51D mutation comprising a reversion mutation that encodes and/or causes the mutant gene to express a gene product comprising a neoantigen amino acid sequence. In particular, the neoantigen amino acid sequence may be as defined in accordance with the first aspect of the invention (e.g. a neoantigen peptide as set forth in Table 5 or Table 6).

In some embodiments the subject is undergoing or is a candidate to undergo therapy with a PARP inhibitor and/or a platinum-based chemotherapeutic.

In some embodiments the method of treatment is a combination therapy that further comprises treatment with an immune checkpoint inhibitor.

In some embodiments the method of treatment is a combination therapy that further comprises treatment with radiotherapy and/or chemotherapy.

In some embodiments the method of treatment is a combination therapy that comprises treatment with both said anti-cancer vaccine and said engineered T cell.

In some embodiments the method of treatment further comprises a step of HLA typing the subject and matching the neoantigen to the HLA allotype of the subject.

In some cases matching the neoantigen to the HLA allotype of the subject comprises a step of predicting MHC class I presentation of the neoantigen sequence by the subject. This may involve use of a computational tool, such as NetMHCpan-4 described in further detail herein.

In a fifth aspect the present invention provides a method for treatment of a proliferative disorder in a mammalian subject in need thereof, comprising administering a therapeutically effective amount of an anti-cancer vaccine of the first aspect of the invention or an engineered T cell of the second aspect of the invention to the subject.

In a sixth aspect, the present invention provide use of an anti-cancer vaccine of the first aspect of the invention or an engineered T cell of the second aspect of the invention in the preparation of a medicament for use in a method of the fifth aspect of the invention.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIGS. 1A-1F—Collation, annotation and standardisation of HR gene reversion mutations. 1A. Common architectures of HR gene reversion mutations associated with platinum or PARPi resistance. 1B. Workflow schematic illustrating the collation, annotation and standardisation of HR gene reversion mutations. 1C. Bar chart illustrating the primary tumour site in 81 patients with HR gene reversions described in the dataset. Patients are stratified by HR gene and by primary tumour site (see colour key). 1D. Bar chart illustrating 231 reversion mutations in the dataset, stratified by HR gene and by primary tumour site. 1E. Bar chart illustrating that the majority of reversion mutations in the dataset arise from patients with different pathogenic mutations. Most patients (80%) had unique pathogenic mutations (annotated as “single-patient” mutations). Reversion cases from multiple patients with common Ashkenazi founder mutations, such as BRCA2:c.6174delT (c. 5946delT in standardised nomenclature) and BRCA1:c.185delAG (c.68_69delAG), were also identified. 1F. Example of unique reversion events observed for multiple patients with a common founder mutation, BRCA2:c.6174delT (c.5496delT), represented on the BRCA2 coding sequence (CDS). Two true reversions to wild-type DNA sequence were observed in two different patients. Second site reversion mutations in other patients are also shown, colored by patient.

FIGS. 2A-2F—Directionality, hot and cold spots for reversion mutations. 2A. Scatter plots showing orientation (5′/upstream or 3′/downstream) of all reversions relative to original pathogenic mutation in BRCA1 (left) or BRCA2 (right). The start and end positions of each reversion mutation (i.e. the start and end of deleted regions) are joined by lines; insertions are not shown. All positions are shown in CDS coordinates. In a few cases deletions extend beyond the plot boundaries, denoted by lines without a terminating point. For the majority of pathogenic mutations, reversion mutations do not have a directional bias and are seen both upstream and downstream of the pathogenic mutation. However, for some pathogenic mutations, e.g. BRCA2 c.5946delT and BRCA2:c7355delA, second site reversions are biased to the DNA sequence downstream of the pathogenic mutation. There is some evidence of a hotspot for reversion mutations at BRCA2 position c. 750-775 (highlighted in grey) and for a desert at the BRCA2 C-terminus (highlighted in blue). Colours of points and lines denote different studies. 2B. Conservation of amino acid sequence in BRCA1 (left) and BRCA2 (right) mapped onto CDS position for BRCA1 and BRCA2, defined by conservation scores (see materials and methods) determined by the alignment of 11 mammalian species. Notable peaks of conservation in BRCA2 are seen in the BRC region and the C-terminal OB and TR2 domains. 2C. Histogram illustrating the frequency of pathogenic mutations in the reversion dataset annotated by CDS position in BRCA1 or BRCA2. Pathogenic mutations are shown in 40-bp bins. Two regions of BRCA2 are highlighted; the candidate reversion hotspot at c. 750-775 (grey) and C-terminal region (blue). 2D. Histogram illustrating the frequency of pathogenic mutations in BRCA1 or BRCA2 in TCGA studies covering breast, ovarian, pancreatic and prostate cancer, plotted as in (C). The distribution of reverting mutations in BRCA1 (shown in (C)) was not significantly different from the distribution of BRCA1 mutations in the TCGA dataset (p=0.21, two-sided Kolmogorov-Smirnov test). The frequency of reversions 3′ to CDS position 7500 of BRCA2 was significantly lower than expected frequency based on TCGA mutation data (p=0.003, permutation test). 2E. Domain structure of BRCA1 and BRCA2 proteins annotated by CDS position. 2F. Bar chart illustrating the frequency of different pathogenic mutation types in the reversion (lower) and compared to frequency in TCGA data (upper).

FIGS. 3A-3F—Microhomology usage in reversion mutations. 3A. Example of a reversion mutation in BRCA2 associated with microhomology (patient 201 from Cruz et al.). The pathogenic G>T substitution mutation (BRCA2 c.145G>T) introduces a premature stop codon (TAA) as shown. The reversion mutation (c.145_168del24) is an in-frame deletion removing the mutated codon (shown in two different alignments). The existence of microhomology at this deletion is illustrated by the ambiguous alignment of the two nucleotides (TA) flanking it—these could be aligned equally well at either end as illustrated. 3B. Bar chart of reversion events classified by type. Reversions occurring via deletion are more frequent in BRCA2 than in BRCA1. 3C. Within deletion mutations, the use of microhomology occurs at a similar frequency in BRCA1 and BRCA2. Reversion mutations are plotted as in (B) for deletions only. 3D. Breakdown of microhomology use at deletions by primary tumour site and gene. 3E. Deletion sizes are generally larger in BRCA2 reversions (p=0.0036, Wilcoxon rank sum test) with evidence of microhomology use. Total length of deleted sequence is shown for each reversion event, broken down by gene and presence of microhomology. The y-axis is truncated; seven mutations with deletions >140 bp are not shown. 3F. BRCA2 reversions use longer lengths of microhomology compared to BRCA1. Frequency distribution of length of microhomology used in BRCA1 (red, left—mode 1 bp) compared with BRCA2 (blue, right—mode 2 bp) plotted for all secondary deletions.

FIGS. 4A-4D—Prediction of HLA-mediated antigen presentation of reversion peptides. 4A. Percentage of individuals predicted to present at least one neopeptide from out-of-frame sequence associated with the listed pathogenic deletion mutations. This sequence will be shared with reversion mutations to some extent depending on the position of the reversion relative to the pathogenic mutation. Common founder mutations are highlighted. 4B. Predicted amino acid sequences from BRCA2:c.5946delT [c.6174delT] reversion events showing retention of out-of-frame sequence in many reversion alleles. The predicted protein sequence for each reversion observed for BRCA2:c.5946delT is shown compared to the wild-type (top) and predicted truncated c.5946delT protein sequence (second row). Sequences deriving from translation of out-of-frame coding sequence are shown in the yellow box. Amino acids are shaded based on their alignment to the wild type sequence. 4C. Computational prediction of HLA (HLA-A, HLA-B, HLA-C) presentation of out-of-frame protein sequences from BRCA2 c.5946delT downstream reversions. Presentation likelihood calculated using NetMHCpan 4.0. The table shows the proportion of individuals in a set of 1,261 from the 1000 genomes project that have an HLA type predicted to present (% rank <0.5) at least one neopeptide (length 8 to 11) associated with the indicated out-of-frame sequence (note that such neopeptides can include one or more WT amino acids upstream of the out-of-frame sequence). 4D. Percentage of individuals predicted to present at least one neopeptide for reverted protein sequences from all published cases of reversion mutations that encode neopeptides.

FIG. 5—Most reversion mutations are unique. Bar graph describing the number of reversion mutations associated with each pathogenic HR-gene mutation described in the dataset. Most pathogenic mutations in the dataset are observed in a single patient (left panel). In general, reversion mutations were unique for a given pathogenic mutation.

FIGS. 6A-6B—6A. Number of reversion mutations for pathogenic mutations represented by multiple patients, plotted as in FIG. 5. B. Example of unique reversion events observed for a common founder mutation BRCA1:c.185delAG (c.68_68delAG), represented on the BRCA1 CDS. Three true reversions to wild-type sequence were observed in two different patients. Second site reversion mutations are also shown, colored by patient.

FIGS. 7A-7B—Reversion mutations often occur at a distance from the original mutation, leading to out-of-frame protein sequence. 7A. Schematic illustrating reversion distance being defined as the minimum distance between the pathogenic and reversion mutation. If the reversion mutation encompasses the pathogenic mutation, the reversion distance will be zero. 7B. Cumulative frequency distribution of reversion distance (in CDS coordinates) using data from all 231 reversion events.

FIG. 8—HLA presentation profile for the BRCA2 RENLSRYQMLHYKTQ (SEQ ID NO: 1247) neo-peptides (most likely peptides to be immunogenic). BR—Best predicted rank among possible neopeptides. WB—weak binding threshold, SB—strong binding threshold. Peptides derived from the sequence shown have a strong binding affinity predicted for many HLA alleles, indicating that they are likely to be presented to the immune system.

FIGS. 9A-9C—Research Plan to assess the therapeutic potential of vaccinations against revertant neoantigens. 9A. Schematic of the T-cell priming assay to assess whether predicted neopeptides are presented as antigens. 9B. Experimental scheme for vaccination experiment to determine whether the immune system can be primed against reverted tumours. 9C. Use of vaccines and immune checkpoint inhibitors (CPI-anti-Pd1 or anti-Ctla4) to determine whether an immune-targeted therapeutic strategy would be feasible.

FIGS. 10A-10B—(10A) Individual level, HLA-matched, predictions demonstrate that reversion neopeptides are likely to bind the HLA type of patients they arise in. For each patient, potential neopeptides were identified in the detected reversion mutations and MHC binding predicted using netMHCpan-4.0 and the HLA type of patient. HLA typing used seq2HLA, Polysolver or PCR—SSO from tumour or blood depending on the data/material available. The number of strong and weak binding predictions (HLA-peptide pairs; Strong: % Rank <0.5, Weak <2.0) is plotted for each patient. 12/14 patients are predicted to present at least one neoantigen; the two without predicted presentation have reversions mediated by single nucleotide variants, all others are compensating frameshifts/deletions. (10B) Pathogenic mutations in patients modelled in FIG. 10A. These results demonstrate that reversions are likely to be presented in the patients in which they arise.

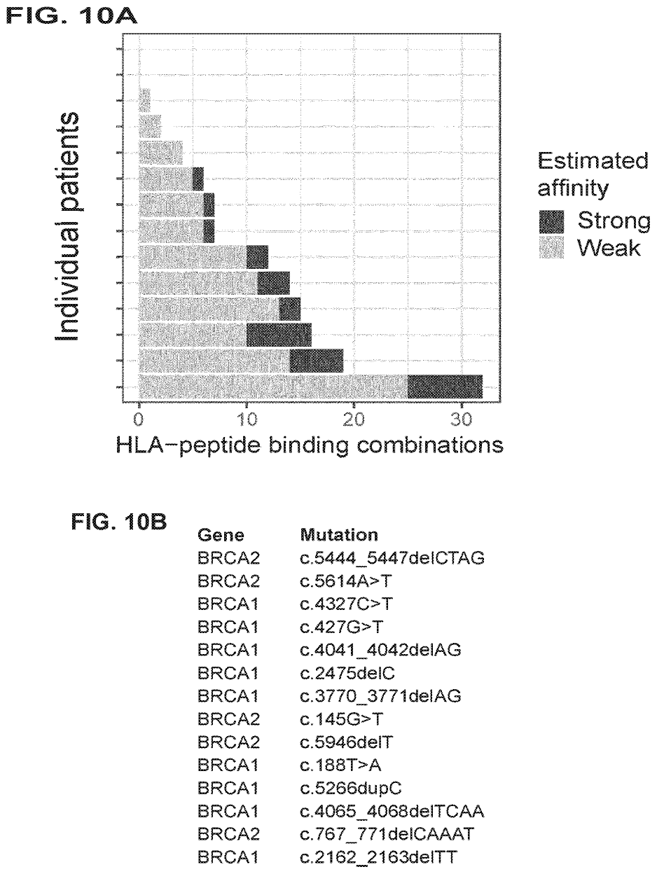

FIGS. 11A-11B—Presentation of predicted reversion neopeptides by HLA-A2:01. (11A) Reversion neopeptides with predicted binding for HLA-A2:01. (11B) T2 cells, which express HLA-A2:01 but are defective in processing and presentation of endogenous peptide antigens, were pulsed with the indicated preprocessed synthetic peptides. Binding of these peptides stabilizes the MHC at the cell surface. Several peptides (GIA . . . , SQM . . . , KIM . . . , SLL . . . ) result in stabilization of cell surface MHC. These results provide direct “wet” experimental evidence that the predicted binding translates into actual binding to the relevant MHC.

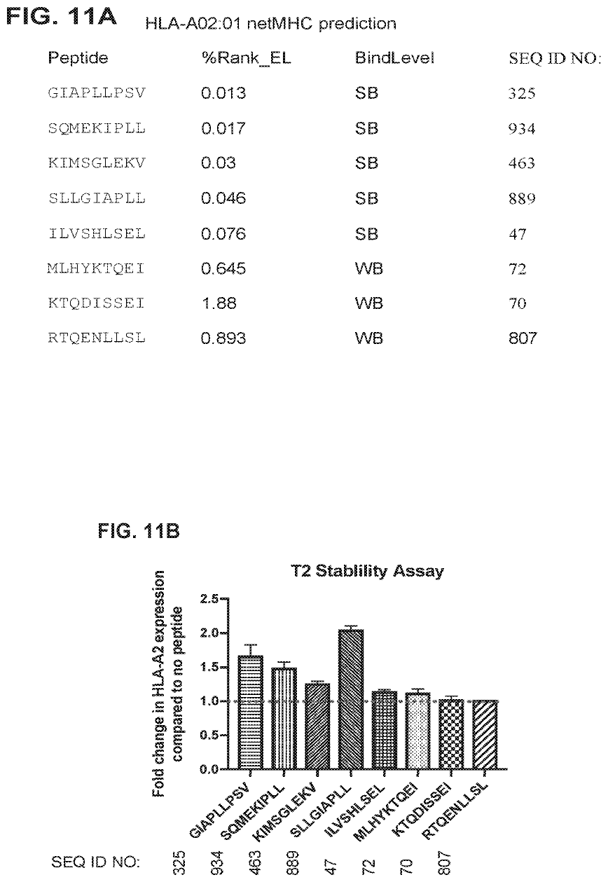

Table 1—Studies describing HR-gene reversion mutations in patients collated for this analysis. Some studies not listed in the table reported mutations in cell lines and PDX (Dréan et al., 2017; Ikeda et al., 2003; Sakai et al., 2009; Ter Brugge et al., 2016; Wang et al., 2016). These are included in the database but not the analysis described in this paper.

Table 2—List of cases in collected studies for which reversions were assessed but not identified.

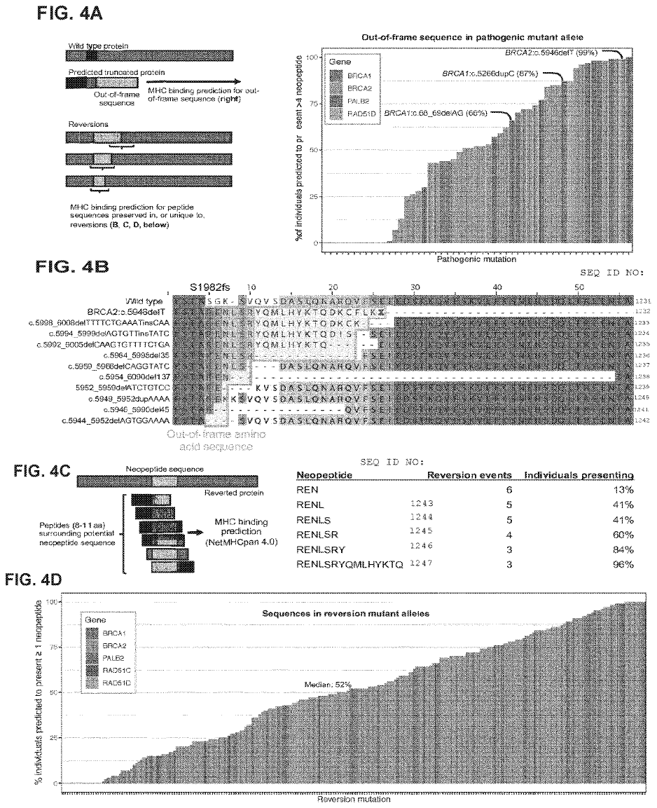

Table 3—NetMHCpan predictions for neopeptides unique to revertant sequences (FIG. 4D).

Table 4—Studies from cBioPortal used for analysis of pathogenic mutations.

Table 5—Selected primary mutations encoding neopeptides. The peptide sequence of each neoantigen is shown together with the number of individuals presenting the peptide sequence, the primary mutations encoding the peptide sequence and the number of primary mutations that encode the peptide sequence.

Table 6—Selected reversion mutations encoding neopeptides. The peptide sequence of each neoantigen is shown together with the number of individuals presenting the peptide sequence, the reversion mutations encoding the peptide sequence and the number of reversion mutations that encode the peptide sequence.

Table 7—Presentation scores of specified reversion mutations. “HLA presentation likelihood” shows the percentage of 1000 genomes individuals.

Table 8—Predicted binding capacity of the predicted mutations.

### DETAILED DESCRIPTION OF THE INVENTION

In describing the present invention, the following terms will be employed, and are intended to be defined as indicated below.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### BRCA1

BRCA1 DNA repair associated or BRCA1 gene encodes a nuclear phosphoprotein that plays a role in maintaining genomic stability, and it also acts as a tumour suppressor. The human BRCA1 gene has the NCBI Gene ID: 672. The genomic sequence of BRCA1 is disclosed at NCBI Reference sequence NC_000017.11>NC_000017.11:c43125364-43044295 Homo sapiens chromosome 17, GRCh38.p13 Primary Assembly. LOCUS NC_000017 81070 bp DNA linear CON 2 Mar. 2020 DEFINITION Homo sapiens chromosome 17, GRCh38.p13 Primary Assembly. ACCESSION NC_000017 REGION: complement (43044295 . . . 43125364) VERSION NC_000017.11. The entire contents of which are incorporated herein by reference. The transcript for BRCA1 is disclosed at NM_007294.3.;

### BRCA2

BRCA2 DNA repair associated or BRCA2 gene encodes a DNA repair protein. The BRCA2 gene has the NCBI Gene ID: 675. The genomic sequence of BRCA2 is disclosed at NCBI Reference sequence >NC_000013.11:32315508-32400268 Homo sapiens chromosome 13, GRCh38.p13 Primary Assembly. LOCUS NC_000013 84761 bp DNA linear CON 2 Mar. 2020 DEFINITION Homo sapiens chromosome 13, GRCh38.p13 Primary Assembly. ACCESSION NC_000013 REGION: 32315508.32400268. VERSION NC_000013.11. The entire contents of which are incorporated herein by reference. The transcript for BRCA2 is disclosed at NM_000059.3

### PALB2

Partner and localizer of BRCA2 (PALB2) is protein which in humans is encoded by the PALB2 gene. The PALB2 gene has the NCBI Gene ID: 79728. The genomic sequence of PALB2 is disclosed at NCBI reference sequence NG_007406 38196 bp DNA linear PRI 3 Jan. 2020 DEFINITION Homo sapiens partner and localizer of BRCA2 (PALB2), RefSeqGene (LRG_308) on chromosome 16 ACCESSION NG_007406 REGION: 5001 . . . 43196 VERSION NG 007406.1. The entire contents of which are incorporated herein by reference. The transcript for PALB2 is disclosed at NM_024675.4.

### CDK12

CDK12 cyclin-dependent kinase 12 (CDK12) is a protein which in humans is encoded by the CDK12 gene. The CDK12 gene has the NCBI Gene ID: 51755. Reversion mutations in the CDK12 gene have been described in Fu et al AACR Meeting 2021, Proceedings of the 112th Annual Meeting of the American Association for Cancer Research; 2021 Apr. 10-15. Philadelphia (PA): AACR; 2021. Abstract 25 in session MS. CL01.01—Biomarkers (incorporated herein by reference).

### RAD51B

RAD51 paralog B is encoded by the RAD51B gene having the NCBI Gene ID: 5890. Reversion mutations in the RAD51B gene have been described in L'heureux et al. Clin Cancer Res 2020 DOI: 10.1158/1078-0432.CCR-19-4121 (incorporated herein by reference).

### RAD51C

RAD51 paralog C (RAD51C) is protein which in humans is encoded by the RAD51C gene. The RAD51C gene has the NCBI Gene ID: 5889. The genomic sequence of RAD51C is disclosed at NCBI reference sequence NG 023199 41770 bp DNA linear PRI 5 Aug. 2019 DEFINITION Homo sapiens RAD51 paralog C (RAD51C), RefSeqGene (LRG_314) on chromosome 17. ACCESSION NG_023199 REGION: 4972 . . . 46741 VERSION NG 023199.1. The entire contents of which are incorporated herein by reference. The transcript for RAD51C is disclosed at NM_002876.3.

### RAD51D

RAD51 paralog D (RAD51D) is protein which in humans is encoded by the RAD51D gene. The RAD51D gene has the NCBI Gene ID: 5892. The genomic sequence of RAD51D is disclosed at NCBI reference sequence NG_031858 20078 bp DNA linear PRI 4 Aug. 2019 DEFINITION Homo sapiens RAD51 paralog D (RAD51D), RefSeqGene (LRG_516) on chromosome 17. ACCESSION NG 031858 REGION: 5001 . . . 25078 VERSION NG 031858.1. The entire contents of which are incorporated herein by reference. The transcript for RAD51D is disclosed at NM_001142571.2

### Sample and Mutation Detection

A sample or “test sample” as used herein may be a cell or tissue sample (e.g. a biopsy), a biological fluid, an extract (e.g. a protein or DNA extract obtained from the subject). In particular, the sample may be a tumour sample. The sample may be one which has been freshly obtained from the subject or may be one which has been processed and/or stored prior to making a determination (e.g. frozen, fixed or subjected to one or more purification, enrichment or extractions steps). When the mutation (e.g. a BRCA1, BRCA2, PALB2, CDK12, RAD51B, RAD51C or RAD51D mutation, whether heterozygous or homozygous) is a germline mutation it may be convenient to use a non-tumour sample (e.g. a cheek swab, blood sample, hair sample or similar DNA-containing sample) to determine the presence or absence of a mutation. When the mutation (e.g. a BRCA1, BRCA2, PALB2, CDK12, RAD51B, RAD51C or RAD51D mutation) is a somatic mutation, for example a mutation that has triggered and/or developed with the cancer, the sample will generally be obtained directly from the tumour, obtained from circulating cancer cells and/or circulating tumour DNA (ctDNA). Techniques for enriching a blood or plasma sample for circulating tumour DNA (e.g. based on fragment size) have been described. Moreover, sequencing techniques for identifying cancer-associated mutations in ctDNA have been described (e.g. based on digital PCR, targeted deep sequencing, nested real-time PCR, and the like). Mutation detection may, for example, comprise sequence alignment between the BRCA1, BRCA2, PALB2, CDK12, RAD51B, RAD51C or RAD51D gene sequence determined for the tumour and the corresponding reference gene sequence, followed by a step of variant calling in which sequence differences (including substitutions, insertions or deletions) are identified. Optionally the corresponding amino acid sequence of the polypeptide encoded by the mutant BRCA1, BRCA2, PALB2, CDK12, RAD51B, RAD51C or RAD51D gene may be derived and used to determine the presence of and/or identity of a neoantigen sequence in the tumour of the subject.

Thus, tumour sequencing, including ctDNA sequencing, may be employed for vaccine selection and/or cell therapy selection, including personalised vaccine and/or personalised cell therapy. Mutation detection based on ctDNA has the advantage of being non-invasive (“liquid biopsy”). In some cases a blood sample may be a source of tumour DNA in the form of plasma-derived ctDNA and a source of germ line DNA, e.g., in the form of buffy coat (comprising leukocytes and platelets). The germ line DNA sequence may be compared with the ctDNA sequence to identify somatic mutations in HR DNA repair genes, such as BRCA1, BRCA2, PALB2, CDK12, RAD51B, RAD51C and RAD51D. Additionally or alternatively, ctDNA-derived sequence reads may be aligned to a wild-type reference sequence to identify mutations in one or more of said HR DNA repair genes. In some cases, a subject may be a carrier of a mutation in in one or more of said HR DNA repair genes, e.g. having a germ line mutation in one or more of the BRCA1, BRCA2, PALB2, CDK12, RAD51B, RAD51C and RAD51D genes. The tumour cells of such a carrier subject may comprise the same germ line mutation(s) and/or different or additional mutations. In some cases, the subject may be a non-carrier of a mutation in in one or more of said HR DNA repair genes, i.e. having a somatic mutation, but not having germ line mutation in one or more of the BRCA1, BRCA2, PALB2, CDK12, RAD51B, RAD51C and RAD51D genes.

### Chimeric Antigen Receptors

Chimeric Antigen Receptors (CARs) are recombinant receptor molecules which provide both antigen-binding and T cell activating functions. CAR structure and engineering is reviewed, for example, in Dotti et al., Immunol Rev (2014) 257 (1), which is hereby incorporated by reference in its entirety.

CARs comprise an antigen-binding domain linked to a transmembrane domain and a signalling domain. An optional hinge domain may provide separation between the antigen-binding domain and transmembrane domain, and may act as a flexible linker.

The antigen-binding domain of a CAR may be based on the antigen-binding region of an antibody which is specific for the antigen to which the CAR is targeted. For example, the antigen-binding domain of a CAR may comprise amino acid sequences for the complementarity-determining regions (CDRs) of an antibody which binds specifically to the target protein. The antigen-binding domain of a CAR may comprise or consist of the light chain and heavy chain variable region amino acid sequences of an antibody which binds specifically to the target protein. The antigen-binding domain may be provided as a single chain variable fragment (scFv) comprising the sequences of the light chain and heavy chain variable region amino acid sequences of an antibody. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64(24): 9160-9166).

The transmembrane domain is provided between the antigen-binding domain and the signalling domain of the CAR. The transmembrane domain provides for anchoring the CAR to the cell membrane of a cell expressing a CAR, with the antigen-binding domain in the extracellular space, and signalling domain inside the cell. Transmembrane domains of CARS may be derived from transmembrane region sequences for CD3-ζ, CD4, CD8 or CD28.

The signalling domain allows for activation of the T cell. The CAR signalling domains may comprise the amino acid sequence of the intracellular domain of CD3-ζ, which provides immunoreceptor tyrosine-based activation motifs (ITAMs) for phosphorylation and activation of the CAR-expressing T cell. Signalling domains comprising sequences of other ITAM-containing proteins have also been employed in CARS, such as domains comprising the ITAM containing region of FcγRI (Haynes et al., 2001 J Immunol 166 (1): 182-187). CARs comprising a signalling domain derived from the intracellular domain of CD3-ζ are often referred to as first generation CARs.

Signalling domains of CARs may also comprise co-stimulatory sequences derived from the signalling domains of co-stimulatory molecules, to facilitate activation of CAR-expressing T cells upon binding to the target protein. Suitable co-stimulatory molecules include CD28, OX40, 4-1BB, ICOS and CD27. CARs having a signalling domain including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1BB-mediated signalling is through TNF receptor associated factor (TRAF) adaptor proteins. Signalling domains of CARs therefore sometimes contain co-stimulatory sequences derived from signalling domains of more than one co-stimulatory molecule. CARs comprising a signalling domain with multiple co-stimulatory sequences are often referred to as third generation CARS.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH₂CH₃ region of immunoglobulin.

### Neoantigen Reactive T Cells (NAR-T)

A neoantigen is a newly formed antigen that has not been previously presented to the immune system. The neoantigen is tumour-specific, which arises as a consequence of a mutation within a cancer cell and is therefore not expressed by healthy (i.e. non-tumour) cells.

The neoantigen may be caused by any non-silent mutation which alters a protein expressed by a cancer cell compared to the non-mutated protein expressed by a wild-type, healthy cell. For example, the mutated protein may be a translocation or fusion.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual. For example, the mutation may be a single nucleotide variant (SNV), multiple nucleotide variants, a deletion mutation, an insertion mutation, a translocation, a missense mutation or a splice site mutation resulting in a change in the amino acid sequence (coding mutation).

The human leukocyte antigen (HLA) system is a gene complex encoding the major histocompatibility complex (MHC) proteins in humans. A neoantigen may be processed to generate distinct peptides which can be recognised by T cells when presented in the context of MHC molecules. A neoantigen presented as such may represent a target for therapeutic or prophylactic intervention in the treatment or prevention of cancer in a subject.

An intervention may comprise an active immunotherapy approach, such as administering an immunogenic composition or vaccine comprising a neoantigen to a subject. Alternatively, a passive immunotherapy approach may be taken, for example adoptive T cell transfer or B cell transfer, wherein a T and/or B cells which recognise a neoantigen are isolated from tumours, or other bodily tissues (including but not limited to lymph node, blood or ascites), expanded ex vivo or in vitro and readministered to a subject.

T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may be co-cultured with antigen-presenting cells (APCs), which may have been irradiated. The APCs may be dendritic cells or B cells. The dendritic cells may have been pulsed with peptides containing the identified neoantigen as single stimulants or as pools of stimulating neoantigen peptides. Expansion of T cells may be performed using methods which are known in the art, including for example the use of artificial antigen presenting cells (aAPCs), which provide additional co-stimulatory signals, and autologous PBMCs which present appropriate peptides. Autologous PBMCs may be pulsed with peptides containing neoantigens as single stimulants, or alternatively as pools of stimulating neoantigens.

### Engineered T Cell

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject.

The cell may be a CD4⁺ T cell or a CD8⁺ T cell. In some embodiments, the cell is a target protein-reactive CAR-T cell. In embodiments herein, a “target protein-reactive” CAR-T cell is a cell which displays certain functional properties of a T cell in response to the target protein for which the antigen-binding domain of the CAR is specific, e.g. expressed at the surface of a cell. In some embodiments, the properties are functional properties associated with effector T cells, e.g. cytotoxic T cells.

In some embodiments, the engineered T cell may display one or more of the following properties: cytotoxicity to a cell comprising or expressing the target protein; proliferation, increased IFNγ expression, increased CD107a expression, increased IL-2 expression, increased TNFα expression, increased perforin expression, increased granzyme B expression, increased granulysin expression, and/or increased FAS ligand (FASL) expression in response to the target protein, or a cell comprising or expressing the target protein. In some embodiments, the engineered T cell expresses an engineered T cell receptor. For example, the engineered T cell may express a cancer-specific T cell receptor, such as the NY-ESO-1 T cell receptor. In embodiments, the engineered T cell does not express an endogenous T cell receptor. In embodiments, the engineered T cell does not express the immune checkpoint molecule programmed cell death protein 1 (PD-1). In embodiments, the engineered T cell has been engineered to remove the endogenous T cell receptor and/or the immune checkpoint molecule programmed cell death protein 1 (PD-1).

The present invention also provides a method for producing an engineered T cell according to the present invention. In some embodiments, the methods are performed in vitro.

In some embodiments, the engineered T cell further comprises an introduced T cell receptor (e.g. a chimeric antigen receptor) that specifically recognises the neoantigen that is encoded by the BRCA1, BRCA2, PALB2, CDK12, RAD51B, RAD51C or RAD51D reversion mutation and which is expressed on or in proximity to a tumour (e.g. tumour stroma). The present invention also provides methods of introducing an isolated nucleic acid or vector encoding the T cell receptor into the T cell. In some embodiments the isolated nucleic acid or vector is comprised in a viral vector, or the vector is a viral vector. In some embodiments, the method comprises introducing a nucleic acid or vector according to the invention by electroporation.

### Compositions

The present invention also provides compositions comprising a vaccine or cell according to the invention.

Vaccines and engineered T cells according to the present invention may be formulated as pharmaceutical compositions for clinical use and may comprise a pharmaceutically acceptable carrier, diluent, excipient or adjuvant.

In accordance with the present invention methods are also provided for the production of pharmaceutically useful compositions, such methods of production may comprise one or more steps selected from: isolating a vaccine or engineered T cell as described herein; and/or mixing with a pharmaceutically acceptable carrier, adjuvant, excipient or diluent.

### Uses of and Methods of Using the Vaccines, Cells and Compositions

The vaccines and engineered T cells and pharmaceutical compositions according to the present invention find use in therapeutic and prophylactic methods. In particular, in the manufacture of a medicament for treating or preventing a disease or disorder.

The present invention also provides a method of treating or preventing a disease or disorder, comprising administering to a subject a therapeutically or prophylactically effective amount of a vaccine or an engineered T cell or pharmaceutical composition according to the present invention.

### Administration

Administration of a vaccine or engineered T cell or composition according to the invention is preferably in a “therapeutically effective” or “prophylactically effective” amount, this being sufficient to show benefit to the subject. The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The vaccines and engineered T cells, compositions and other therapeutic agents, medicaments and pharmaceutical compositions according to aspects of the present invention may be formulated for administration by a number of routes, including but not limited to, parenteral, intravenous, intra-arterial, intramuscular, subcutaneous, intradermal, intratumoural and oral. The vaccine peptides, nucleic acids, vectors, cells, composition and other therapeutic agents and therapeutic agents may be formulated in fluid or solid form. Fluid formulations may be formulated for administration by injection to a selected region of the human or animal body, or by infusion to the blood. Administration may be by injection or infusion to the blood, e.g. intravenous or intra-arterial administration.

Administration may be alone or in combination with other treatments, either simultaneously or sequentially dependent upon the condition to be treated.

In some embodiments, treatment with a vaccine or engineered T cell or composition of the present invention may be accompanied by other therapeutic or prophylactic intervention, e.g. chemotherapy, immunotherapy (including immune checkpoint inhibitor therapy), radiotherapy, surgery, and/or hormone therapy.

In some embodiments the other therapeutic or prophylactic intervention may comprise a PARP inhibitor (e.g. one or more of Olaparib, Rucaparib, Niraparib, Talazoparib, Veliparib, BGB-290 (Pamiparib), CEP 9722 and E7016), an ATR (ataxia-telangiectasia and Rad3 related) inhibitor (e.g. one or more of NU6027, NVP-BEZ235, VE-821, VE-822, AZ20, and AZD6738—see Weber and Ryan, Pharmacology & Therapeutics, 2015, Vol. 149, pp. 124-138, incorporated herein by reference), an ATM (ataxia-telangiectasia mutated) inhibitor (e.g. one or more of CP-466722, KU-55933, KU-60019 and KU-559403—see Weber and Ryan, Pharmacology & Therapeutics, 2015, Vol. 149, pp. 124-138, incorporated herein by reference), a DNA-dependent protein kinase (DNA-PK) inhibitor (e.g. one or more of LY294002, NU7441, KU-0060648—see Mohiuddin and Kang, Front. Oncol., 2019, Vol. 9, 635, doi: 10.3389/fonc.2019.00635) and/or a DNA polymerase theta (DNA POLQ) inhibitor (e.g. a heterocyclic substituted urea as disclosed in WO2020/030925, incorporated herein by reference and/or a thiazoleurea as disclosed in WO2020/030924, incorporated herein by reference).

Simultaneous administration refers to administration of the vaccine, engineered T cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays).

The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoural.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumour antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu (erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inihibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: a PARP inhibitor (e.g. one or more of Olaparib, Rucaparib, Niraparib, Talazoparib, Veliparib, BGB-290 (Pamiparib), CEP 9722 and E7016), an ATR (ataxia-telangiectasia and Rad3 related) inhibitor (e.g. one or more of NU6027, NVP-BEZ235, VE-821, VE-822, AZ20, and AZD6738—see Weber and Ryan, Pharmacology & Therapeutics, 2015, Vol. 149, pp. 124-138, incorporated herein by reference), an ATM (ataxia-telangiectasia mutated) inhibitor (e.g. one or more of CP-466722, KU-55933, KU-60019 and KU-559403—see Weber and Ryan, Pharmacology & Therapeutics, 2015, Vol. 149, pp. 124-138, incorporated herein by reference), a DNA-dependent protein kinase (DNA-PK) inhibitor (e.g. one or more of LY294002, NU7441, KU-0060648—see Mohiuddin and Kang, Front. Oncol., 2019, Vol. 9, 635, doi: 10.3389/fonc.2019.00635) and/or a DNA polymerase theta (DNA POLQ) inhibitor (e.g. a heterocyclic substituted urea as disclosed in WO2020/030925, incorporated herein by reference and/or a thiazoleurea as disclosed in WO2020/030924, incorporated herein by reference).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-UR, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXomeR, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte-Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron AR (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-AR (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

### Immune Checkpoint Inhibitor

Immune checkpoint inhibitors include inhibitors of PD-1 (e.g. Nivolumab, Pembrolizumab and BGB-A317), inhibitors of PD-L1 (e.g. atezolizumab, avelumab and durvalumab) and inhibitors of CTLA-4 (e.g. ipilimumab). As described herein, treatment with immune checkpoint inhibitor therapy is expected to be particularly beneficial for cancers that have one or more mutant HR DNA repair genes (e.g. BRCA1, BRCA2, PALB2, CDK12, RAD51B, RAD51C or RAD51D) comprising reversion mutations that result in neoantigen sequence capable of eliciting an immune response. In particular, combination therapy with a vaccine or engineered T cell of the present invention with immune checkpoint inhibitor therapy is expected to combat immune escape by the tumour and render the anti-cancer therapy more effective.

### Cancer

In some embodiments, the disease or disorder to be treated or prevented in accordance with the present invention is a cancer.

The cancer may be any unwanted cell proliferation (or any disease manifesting itself by unwanted cell proliferation), neoplasm or tumour or increased risk of or predisposition to the unwanted cell proliferation, neoplasm or tumour. The cancer may be benign or malignant and may be primary or secondary (metastatic). A neoplasm or tumour may be any abnormal growth or proliferation of cells and may be located in any tissue. Examples of tissues include the adrenal gland, adrenal medulla, anus, appendix, bladder, blood, bone, bone marrow, bowel, brain, breast, cecum, central nervous system (including or excluding the brain) cerebellum, cervix, colon, duodenum, endometrium, epithelial cells (e.g. renal epithelia), eye, germ cells, gallbladder, oesophagus, glial cells, head and neck, heart, ileum, jejunum, kidney, lacrimal glad, larynx, liver, lung, lymph, lymph node, lymphoblast, maxilla, mediastinum, mesentery, myometrium, mouth, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Without wishing to be bound by theory, it is believed that immune dysfunction may enable the progression of any type of cancer since most cancers exist in the context of the host's immune system. Indeed, most cancers are at least initially recognised and attacked by the immune system, and eventually able to progress through tumour-mediated immunosuppression and tumour evasion mechanisms. Examples of cancer to treat may be selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (gliomas, astrocytomas, glioblastomas), melanoma, lymphoma, small bowel cancers (duodenal and jejunal), leukemia, pancreatic cancer, hepatobiliary tumours, germ cell cancers, prostate cancer, head and neck cancers, thyroid cancer and sarcomas. The present invention is likely to be particularly useful in the context of treatment of cancers including high grade serous ovarian cancer (HGSOC), triple-negative breast cancer (TNBC), castrate resistant metastatic prostate cancer and pancreatic cancer.

Further, the present invention is likely to be particularly useful in the context of treatment of cancers that have a high neoantigen load. A cancer may be predicted to have high neoantigen load if it has high tumour mutational burden, which can be quantified by measuring the somatic mutation prevalence (number of somatic mutations per megabase of tumour genome) for a sample or plurality of samples. Somatic mutation prevalence for various cancer types have been quantified in Alexandrov et al. (Nature volume 500, pages 415-421 (2013)). Cancer types that have high tumour mutational burden may include those with a median numbers of somatic mutations per megabase of at least 1, at least 5, or at least 10. For example, melanomas and squamous lung cancers are typically considered to have high mutational burden.

The present invention is likely to be particularly useful for the treatment of a tumour that has acquired or is predicted to be likely to acquire or show resistance to PARP inhibitor therapy or platinum therapy. Examples of PARP inhibitor therapy include: Olaparib, Rucaparib, Niraparib, Talazoparib, Veliparib, BGB-290 (Pamiparib), CEP 9722 and E7016. Platinum-based chemotherapeutic agents include: cisplatin, carboplatin, oxaliplatin, nedaplatin, triplatin tetranitrate, phenanthriplatin, picoplatin, and satraplatin.

Tumours to be treated may be nervous or non-nervous system tumours. Nervous system tumours may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumours may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer (e.g. small cell), colon cancer, ovarian cancer, pancreatic cancer, thymic carcinoma, NSCLC, haematologic cancer and sarcoma.

### Adoptive Transfer

In embodiments of the present invention, a method of treatment or prophylaxis may comprise adoptive transfer of immune cells, particularly T cells. Adoptive T cell transfer generally refers to a process by which T cells are obtained from a subject, typically by drawing a blood sample from which T cells are isolated. The T cells are then typically treated or altered in some way, optionally expanded, and then administered either to the same subject or to a different subject. The treatment is typically aimed at providing a T cell population with certain desired characteristics to a subject, or increasing the frequency of T cells with such characteristics in that subject. Adoptive transfer of CAR-T cells is described, for example, in Kalos and June 2013, Immunity 39(1): 49-60, which is hereby incorporated by reference in its entirety.

In the present invention, adoptive transfer is performed with the aim of introducing, or increasing the frequency of, target protein-reactive T cells in a subject, in particular target protein-reactive CD8⁺ T cells.

In some embodiments, the subject from which the T cell is isolated is the subject administered with the modified T cell (i.e., adoptive transfer is of autologous T cells). In some embodiments, the subject from which the T cell is isolated is a different subject to the subject to which the modified T cell is administered (i.e., adoptive transfer is of allogenic T cells).

The at least one T cell modified according to the present invention can be modified according to methods well known to the skilled person. The modification may comprise nucleic acid transfer for permanent or transient expression of the transferred nucleic acid.

In some embodiments the method may comprise one or more of the following steps: taking a blood sample from a subject; isolating and/or expanding at least one T cell from the blood sample; culturing the at least one T cell in in vitro or ex vivo cell culture; engineering the at least one T cell to insert a modified T cell receptor or CAR, or a nucleic acid, or vector encoding the modified T cell receptor or CAR; expanding the at least one engineered T cell, collecting the at least one engineered T cell; mixing the engineered T cell with an adjuvant, diluent, or carrier; administering the engineered T cell to a subject.

In embodiments according to the present invention the subject is preferably a human subject. In some embodiments, the subject to be treated according to a therapeutic or prophylactic method of the invention herein is a subject having, or at risk of developing, a cancer, e.g. a cancer having a BRCA1, BRCA2, PALB2, CDK12, RAD51B, RAD51C or RAD51D reversion mutation giving rise to expression of the neoantigen.

In some embodiments, the method additionally comprise therapeutic or prophylactic intervention for the treatment or prevention of a disease or disorder, e.g. chemotherapy, immunotherapy, radiotherapy, surgery, vaccination and/or hormone therapy. In some embodiments, the method additionally comprises therapeutic or prophylactic intervention, for the treatment or prevention of a cancer.

### T Cell Therapy

T cell therapy can include adoptive T cell therapy, tumour-infiltrating lymphocyte (TIL) immunotherapy, autologous cell therapy, engineered autologous cell therapy (eACT), and allogeneic T cell transplantation.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumours. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in US2013/0287748, which is herein incorporated by references in its entirety.

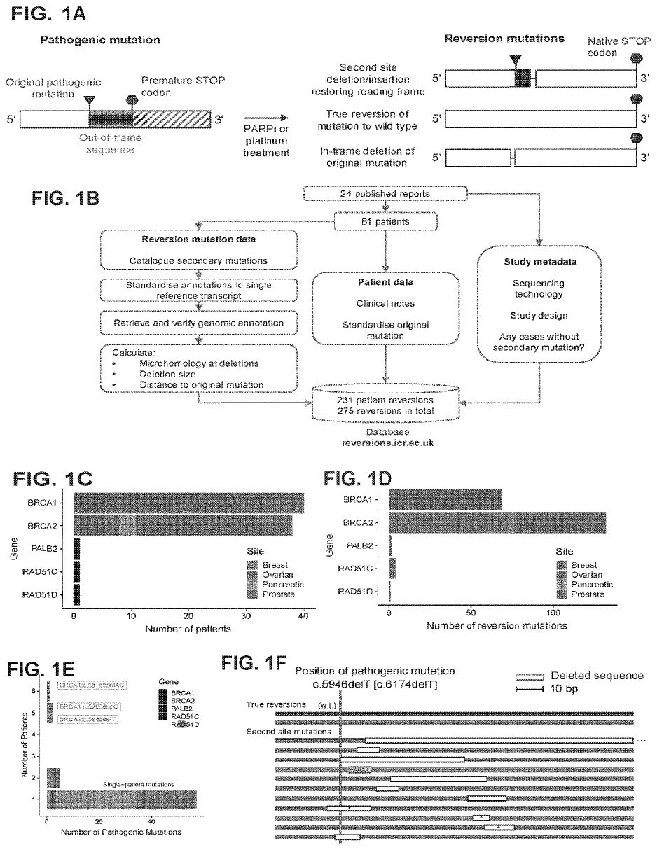

The term “engineered Autologous Cell Therapy,” which can be abbreviated as “eACT™,” also known as adoptive cell transfer, is a process by which a patient's own T cells are collected and subsequently genetically altered to recognize and target one or more antigens expressed on the cell surface of one or more specific tumour cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumour antigen linked to an intracellular signalling part comprising a costimulatory domain and an activating domain. The costimulatory domain can be derived from, e.g., CD28, and the activating domain can be derived from, e.g., CD3-zeta (FIG. 1). In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in US2013/0287748, US2014/0227237, US2014/0099309, and US2014/0050708, and these references are incorporated by reference in their entirety.

### Subjects

The subject to be treated according to the invention may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, may be suspected of having such a disease or condition, or may be at risk from developing such a disease or condition. In particular, the subject may have an HR deficient cancer. The subject may be undergoing or be a candidate for PARP inhibitor therapy or platinum-based therapy. The subject may have wild-type germ-line BRCA1, BRCA2, PALB2, CDK12, RAD51B, RAD51C or RAD51D gene sequence. Alternatively, the subject may be homozygous or heterozygous for a mutation in his or her germ-line gene sequence of BRCA1, BRCA2, PALB2, CDK12, RAD51B, RAD51C or RAD51D (i.e. be a BRCA1, BRCA2, PALB2, CDK12, RAD51B, RAD51C and/or RAD51D mutation carrier). The subject may have a somatic mutation in BRCA1, BRCA2, PALB2, CDK12, RAD51B, RAD51C and/or RAD51D identified in the tumour sample, or in circulating tumour DNA (ctDNA).

The following is presented by way of example and is not to be construed as a limitation to the scope of the claims.

### EXAMPLES

### Materials and Methods

### Collation, Annotation and Standardisation of Reversion Mutations

Studies for this analysis were collated by searching the PubMed database for BRCA1, BRCA2, RAD51C, RAD51D or PALB2 and “Secondary Mutation” or “Reversion”. These studies, or others referenced in these papers, describing mutations in cell lines, patients or PDX models were included. In addition, some cases of reversions discovered as part of a phase I clinical trial that included patients that had progressed on PARP inhibitor or platinum treatment (Yap et al. Cancer Discov. 2020 October; 10 (10): 1528-1543. doi: 10.1158/2159-8290.CD-20-0163) were included.

To aid with the overall analysis, a single transcript was used to annotate all the mutations for a gene. Where sequence information was available in the original publication this was used to annotate the mutation, otherwise the reported annotations were checked for correspondence with the reference transcript chosen for each gene. The original annotation in the publication is provided for cross-referencing purposes, along with patient or case identifiers where used in the published paper. If no case/patient identifiers were used in the original publication, these were constructed for the purposes of this analysis based on the study and sequentially-numbered reversion events. In the database both forms of annotation for the original mutation, the secondary mutations and the chromosomal location (where available) were listed. Where a chromosomal location was not annotated in the original report, this was back-calculated from the CDS annotation using the Ensembl Variant Effect Predictor (VEP, (McLaren et al., 2016)).

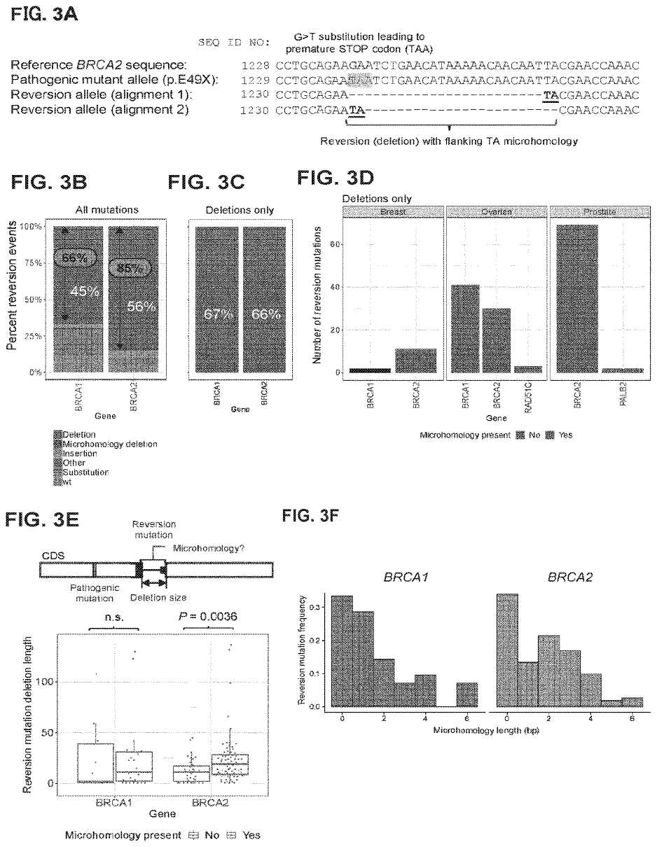

Once the original and secondary mutations are mapped for each case, the distance between the mutations was calculated, noting evidence of microhomology use. The distance between the original mutation and the secondary reversion was measured as the shortest distance, specifically the bases between the last base of one mutation and the first base of the other. Where the secondary reversions are deletions that span the original mutation, the distance was recorded as zero. Mutations with evidence of microhomology use have also been annotated (FIG. 3A), requiring at least one base pair homology. Microhomology is not reported for complex mutations such as insertion-deletions.

The transcripts used for codified annotations were: BRCA1, NM_007294.3; BRCA2, NM_000059.3; RAD51C, NM_058216.2; RAD51D, NM_002878.3 and PALB2, NM_024675_3. Genomic coordinates (hg38) were retrieved using the HGVS CDS annotation on the transcripts above via the Ensembl VEP (Yates et al., 2016). In annotations of the original pathogenic mutation deletions in repetitive regions were aligned to the 3′ end of the deletion, and small insertion were aligned as duplications where appropriate, in order to ensure compatibility with annotations in the BRCA exchange database. Reversion mutation alleles were annotated relative to the reference sequence, including the original pathogenic mutation where this was retained. Secondary deletions that encompassed or were immediately adjacent to the pathogenic mutation (or an alternative valid annotation of the pathogenic mutation) were annotated as a single deletion relative to the reference sequence.

The database recorded reversion mutations on a “per-event” basis, an event being a single observation of a reversion mutation in a patient with a pathogenic mutation in an HR gene. Where individual patients possessed multiple, distinct, reversions (as seen in 29 (36%) of patients described in the database), each reversion was recorded as a different event. In addition, clinical information was recorded, including, where available, information pertaining to cancer type, stage and treatment history (FIG. 1B).

Mutation Data from Tumour Sequencing Studies

BRCA1 and BRCA2 mutation data were retrieved from cBioPortal from the studies listed in Table 4. These studies were chosen to approximately reflect the composition of histologies in the revertant dataset. Mutations were filtered to remove benign variants, but variants of unknown significance were retained.

### Conservation Analysis

Multiple sequence alignments of BRCA1 and BRCA2 orthologues across 11 mammalian species were downloaded from EGGNOG (Powell et al., 2014) and visualised using JalView. Sequences with large gaps relative to the human protein were removed and a consensus score generated (Livingstone and Barton, 1993).

### HLA-Presentation Score Predictions

Given a gene and a mutational event (primary or reversion), an in-house python script was used to generate all peptides associated with the mutation(s). For primary events, the set A of all non-WT peptides associated with the primary mutation was generated (FIG. 4C); for reversions the set B of all non-WT peptides associated to the reversion that are not in A was generated (FIG. 4D. The Best Rank (BR) HLA-presentation score of the mutation was then calculated with respect to each HLA allotype in a list of 195 HLA-A/-B allotypes total found among 1,261 individuals in 1000 Genomes (Gourraud et al., 2014). The BR was defined by predicting the eluted ligand likelihood percentile rank for each peptide associated to the mutation using the program NetMHCpan-4.0 (Jurtz et al., 2017) and taking the minimum elution rank among all peptides (Marty et al., 2017), excluding those with a wild-type NetMHC predicted Icore (Punta et al., 2019). An individual's best rank (IBR) for a mutation m was defined as the minimum BR of the mutation across all HLA allotypes of the individual. The percentage of individuals in 1000 Genomes for which IBR<0.5 were also calculated.

### Data Availability

All data used in this study, along with updated analysis including any cases reported in future, are available to download from reversions.icr.ac.uk.

### Example 1—Collation, Review and Codification of Cases of HR-Gene Reversion Mutation

In order to collate all of the available data on HR-gene reversions associated with PARPi or platinum resistance (FIG. 1A), the literature was searched (see Materials and Methods) up until 1st November 2019, identifying 24 publications which, when combined with some unpublished observations (Yap, T A et al.), described 231 reversion mutation events from a total of 81 patients (Table 1). The majority of patient-derived reversion mutations were in BRCA1 (n=91, 39%) or BRCA2 (n=133, 58%). Relevant studies identifying reversion mutations in tumour cell lines and patient-derived xenografts (PDX) were also included. The number of cases of PARPi or platinum resistance that are not explained by reversion mutations was difficult to determine, as there will be many unreported cases where a reversion is not detected, not investigated or cannot be ruled out. Across all the studies collated, a total of 111 patients with recurrent or platinum/PARPi resistant cancer where the presence of reversion mutations was assessed but not detected were identified (Table 2).

Differences in nomenclature and annotation exist between publications. This often arises from the use of historical mutation nomenclature for BRCA1/2, and/or the varied use of either transcript-based or coding sequence (CDS)-based numbering across different studies. In addition, the nucleotide-based annotation of microhomologies at reversion deletions lacks a standard definition. Given this, all published reversion mutations were reannotated and codified, both in terms of nucleotide change and microhomology use (see Materials and Methods and FIG. 1B). In addition, the clinical information provided for all reported cases was reviewed. All of this information was collated as a singular, freely accessible, database (http://reversions.icr.ac.uk).

In terms of disease subtype, the largest number of revertant cases were from patients with ovarian cancer (131 reversion events from 58 patients; FIG. 1C, D). Rather than reflecting a greater propensity for ovarian cancers to exhibit reversion mutations, the number of ovarian cancers in the collated dataset might reflect the longer period over which PARPi and platinum treatments have been in routine use in this disease. The 81 patients collected in this study possessed 65 different pathogenic HR-gene mutations, the vast majority being in BRCA1 (41 patients with 28 mutations), or BRCA2 (39 patients with 34 mutations) with one case each for PALB2, RAD51C and RAD51D (FIG. 1C). For the majority (78%) of patients, the pathogenic HR gene mutation was a confirmed germline mutation. Two patients (Lin 2018 SubjectID_63 and Carneiro 2018 Patient 1 in the database) had two different pathogenic alleles with reversions in each.

### Example 2—Reversion Mutations are Frequently Unique Events

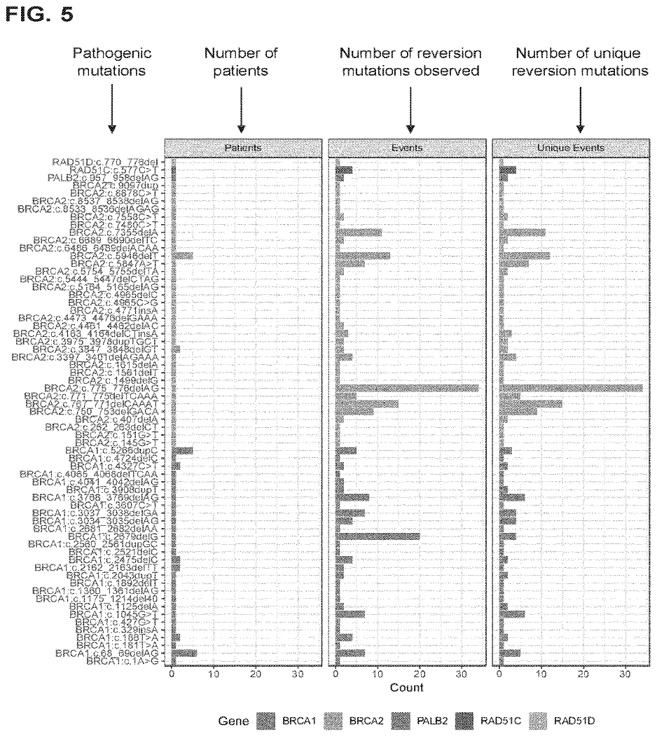

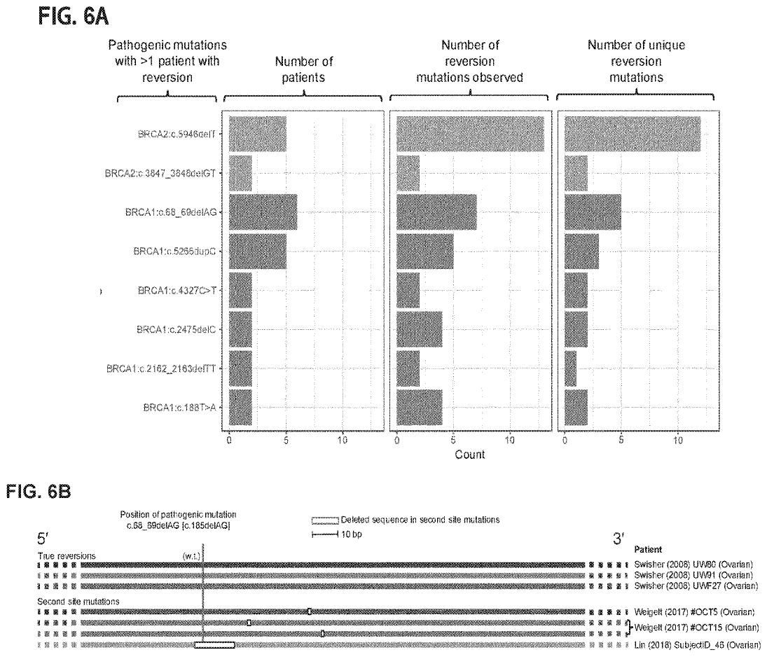

Amongst the 81 patients data was collated from, most (65/81, 80%) had unique pathogenic mutations (FIG. 1E, annotated as “single-patient mutations” and FIG. 5). There were eight pathogenic mutations represented by multiple patients in the dataset, including common founder mutations such as BRCA2:c. 6174delT (c.5946delT in the codified annotation, five patients in the dataset) and BRCA1:c.185delAG (c.68_69delAG, six patients in the dataset; FIG. 1E, FIG. 6A). Even where patients had the same founder pathogenic mutation, the DNA sequences of the reversion mutations that emerged in these patients were all unique, with the exception of true reversions to wild-type, suggesting that there is not a strong propensity for any particular reversion mutation to arise from a particular pathogenic mutation (FIG. 1E, FIG. 5). True wild-type reversions were observed for the BRCA1:c.68_69delAG (n=3) and BRCA2:c.5946delT (n=2) pathogenic mutations (FIG. 1F, FIG. 6B). This could be an intrinsic characteristic of these mutations or a consequence of how these true reversions were identified (Sanger sequencing and haplotype phasing (Norquist et al., 2011; Swisher et al., 2008)). Detection of these true reversions requires relatively long DNA reads to enable these to be phased accurately with a nearby variant.

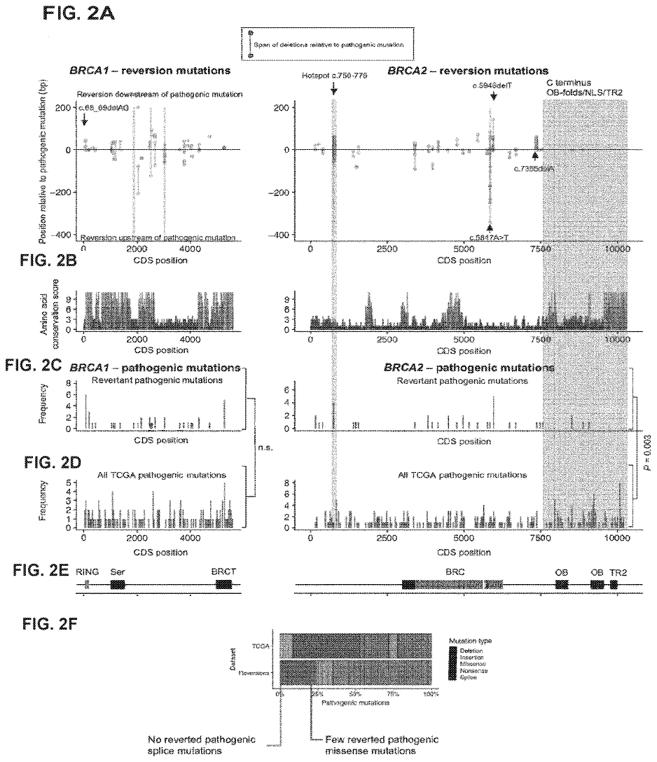

For each of these common founder mutations, the inventors noted that the reversions that emerged in these patients were generally localised to the 3′ flanking sequence of the original pathogenic mutation (transcriptionally downstream, FIG. 1F, FIG. 6B). Several other sites in both BRCA1 and BRCA2 exhibited a predominant directionality in the deletion reversions that were associated with them (e.g. BRCA2:c.7355delA, FIG. 2A, B). However, other pathogenic mutations in BRCA1 or BRCA2 had reversion deletions that occurred on either side of the pathogenic mutation, suggesting that this was not a universal property, but specific to certain pathogenic mutations.

Without being bound by theory, one possible explanation for the directionality of some reversion mutations is that there is critical amino acid sequence encoded by the DNA upstream of the pathogenic mutation that cannot be disrupted if a productive reversion allele is to be formed. However, the inventors did not find any evidence for particular evolutionary conservation of the amino acid residues immediately upstream of the pathogenic mutation, as assessed by Conservation Score (see Materials and Methods, FIG. 2B). Another possible consideration is the position of out-of-frame stop codons relative to the pathogenic mutation, which constrains where productive reversions can occur.

### Example 3—Reversion Mutations in BRCA2 Exhibit Position Dependence

Although the reversion events that emerged in patients with the same founder pathogenic mutations tended to be unique, it was assessed whether the propensity of a pathogenic mutant allele to acquire reversion mutations might depend on its position in either BRCA1 or BRCA2. To do this, the CDS positions and distribution of pathogenic BRCA-gene mutations known to revert (i.e. those in the reversion dataset) were compared to the CDS positions and distribution of likely pathogenic BRCA-gene mutations in TCGA cancer resequencing studies covering ovarian, prostate and breast cancers—the predominant tumour types in our reversion dataset. In the case of BRCA1 mutations, the pathogenic mutations in the reversion dataset were distributed fairly evenly throughout the BRCA1 coding sequence, suggesting that reversion mutation is a possible resistance mechanism for pathogenic mutations at most positions (FIG. 2C) and their distribution was not significantly different from the distribution of BRCA1 mutations in the TCGA dataset (FIG. 2D, p=0.21, two-sided Kolmogorov-Smirnov test).

In contrast to BRCA1, the distribution of BRCA2 reversion mutation positions was less evenly distributed (FIG. 2A). Despite pathogenic truncating mutations in the C-terminal region of BRCA2 being relatively common in large-scale tumour sequencing studies (31% of the pathogenic mutations in the TGCA dataset occurring 3′ to CDS position 7500, FIG. 2D), reversions of pathogenic mutations in this region were rare (FIG. 2C; p=0.003, permutation test). All but one of the reversions in this “desert” region were true reversions to wild-type, or missense mutations rather than deletions. This region of BRCA2 encodes the oligonucleotide/oligosaccharide binding (OB) folds, the nuclear localisation signal (NLS) and TR2 domains known to be required for HR activity (Esashi et al., 2007). Without wishing to be bound by theory, the inventors believe that this distortion in the reversion distribution might suggest that pathogenic mutations in the C-terminal coding sequence of BRCA2 are less able to be productively reverted by second site mutations, particularly deletions, possibly because the surrounding sequence is critical for HR function. This theory was consistent with the known importance of the C terminus for HR function (Esashi et al., 2007) and the high degree of amino acid sequence conservation in this region (FIG. 2B).

In contrast to the reversion “desert” at the C-terminus of BRCA2, the inventors noted a large number of reversion mutations in the N-terminal c.750-775 region (61 reversions in total from four patients in four separate studies, FIG. 2A). These reversions were identified by ctDNA sequencing, which might be more effective in identifying more reversion events per patient than, for example, the bulk sequencing of tumour cells from a solid tumour biopsy (Quigley et al., 2017). However, these mutations originated from four different patients, and this region of BRCA2 did not show a high frequency of pathogenic mutations in the TCGA dataset (FIG. 2D). Without being bound by theory, the inventors theorised that BRCA2 mutations in this region might show a greater propensity to acquire reversions and/or better tolerate the local disruption of the coding sequence in the reverted BRCA2 allele. Consistent with this theory, compared to the C-terminus of BRCA2, the c. 750-775 region is not a highly-conserved region of the protein (FIG. 2B).

### Example 4—Reversion of Pathogenic Missense Mutations is Rare

Multiple types of known pathogenic BRCA1 and BRCA2 mutation exist, including frameshift or nonsense mutations, as well as well-characterised missense and splice site mutations (Cline et al., 2018; Futreal et al., 1994; Lancaster et al., 1996; Landrum et al., 2017). The inventors therefore investigated whether the propensity of a BRCA-gene mutation to acquire reversion mutations might depend on the nature of the pathogenic mutation. Of the 65 pathogenic mutations in the reversion dataset, 40 were present in the BRCA Exchange database of reported mutations (Cline et al., 2018). All of these 40 mutations were classified as pathogenic by the ENIGMA (Spurdle et al., 2011) or ClinVar (Landrum et al., 2017) criteria. All remaining mutations (n=25) without an entry in the BRCA Exchange database were frameshift or nonsense mutations and therefore would be predicted as pathogenic.

Interestingly, very few missense pathogenic mutations in the set of reported reversions were noted. For example, in the TCGA tumour resequencing datasets used previously, 8.6% (8/93) of the known or likely pathogenic BRCA1/2 mutations were missense variants; conversely in the reversion dataset, only a single patient with a missense mutation (BRCA1:p.C61S missense mutation, known to be pathogenic) was present (FIG. 2F). A revertant patient with a BRCA1 p.M1I pathogenic mutation, which would, in its non-reverted state, result in loss of the translation start site was also noted. In each of these cases, the reversion seen was a true reversion to wild-type. Moreover, there were no splice-site pathogenic mutations among the reversion cases. A similar observation had been previously made in an analysis of the ARIEL2 clinical trial assessing the efficacy of the PARPi, rucaparib, in relapsed, platinum-sensitive high-grade ovarian carcinomas; out of a cohort of 112 patients, four had BRCA-gene missense mutations and ten possessed splice-site mutations. No reversions were found in any of these 14 patients, five of which were platinum resistant or refractory at the start of the study (Lin et al., 2019). One explanation for this relative paucity of reversions from tumours with pathogenic missense BRCA-gene mutations could be that missense variants affect individual amino acid residues that are critical for BRCA1/2 function; such mutations may thus be less likely to revert productively by deletion, since this would also render the “reverted” protein non-functional.

### Example 5—Microhomology Use in Reversions is Frequent but not Universal

When BRCA2 reversion mutations were originally identified in cultured tumour cell lines, each of the deletion-mediated second site reversion events was characterised by the presence of DNA sequence microhomology at the ends of deleted regions (Edwards et al., 2008; Sakai et al., 2009; Sakai et al., 2008). Without being bound by theory, the inventors theorised that DNA repair processes that exploit regions of microhomology to repair DSBs could be responsible for the reversion events. From a mechanistic perspective, the loss of homologous recombination is known to cause increased use of MMEJ (Yun and Hiom, 2009), suggesting that the microhomology-characterised reversions could even be a downstream effect of the loss of HR (Edwards et al., 2008). In subsequent reports of HR-gene reversion in patients, microhomology was also a frequent feature of reversions mediated by deletion, an observation that extended beyond BRCA1 or BRCA2 reversion, to reversion events in PALB2, RAD51C and RAD51D (Barber et al., 2013; Edwards et al., 2008; Goodall et al., 2017; Kondrashova et al., 2017; Norquist et al., 2011; Patch et al., 2015; Quigley et al., 2017; Sakai et al., 2008; Swisher et al., 2008). Therefore, to better understand the aetiology of reversion mutations, the use of microhomology was assessed for the reversion events in the dataset. Such events can be recognised via their ambiguous alignments to the reference sequence, as the bases immediately adjacent to the deletion can be aligned equally well at either side of the deletion (FIG. 3A, alignment 1 and 2). Surprisingly, when all of the reported reversion events were systematically assessed, the use of microhomology mediated deletions was clearly not universal. Only 51% (106 of 205 with sequence information) of the reversion cases across the whole dataset were deletions that had evidence of microhomology. In cases of BRCA1 reversion, only 45% showed evidence of microhomology use; for BRCA2 reversions, only 56% showed microhomology use (FIG. 3B).

Overall, 66% of the BRCA1 reversions were mediated by deletions compared to 85% for BRCA2 (categories “deletion” and “microhomology deletion” in FIG. 3B). Therefore, the inventors theorised that BRCA1 mutant cells may use a wider range of pathways of DNA repair that lead to substitution or true wild-type reversions compared to BRCA2, where most events are deletion-mediated (FIG. 3B). When considering only reversions mediated by deletion, the fraction for which microhomology was present was similar between BRCA1 (67%) and BRCA2 (66%), but still approximately one third of deletions in each case did not exhibit microhomology (FIG. 3C). This suggested that DNA repair or mutagenic processes that do not utilise regions of DNA microhomology could also play a major role in the formation of reversion deletion mutations in patients.

There may also be primary tumour site differences in the use of microhomology. Microhomology use was rarely observed in breast cancer reversion cases in the dataset compared with reversions in ovarian or prostate cancers (FIG. 3D); however, the numbers in these subgroups are small and based on limited numbers of studies.

### Example 6—Characteristics of Reversion Mutations Indicate Strong Selective Pressure for Close to Full-Length Proteins

BRCA2 reversion mutations identified in cell line models were often large intragenic deletions (>50 kb in some cases) that removed large segments of the coding sequence despite restoring the open reading frame of the gene and leading to expression of the C-terminal NLS and OB/TR2 domains (Edwards et al., 2008). This might suggest that much of the BRCA2 coding sequence, with the exception of the C-terminus, is dispensable for tolerance of PARPi or platinum, at least in cultured cells. For BRCA1, cell line-based studies suggest that much of the protein coded for by exon 11 (1142 amino acids, 60% of the coding sequence) is dispensable for therapy resistance (Wang et al., 2016). However, and in contrast to the observations in pre-clinical models (Edwards et al., 2008), the intragenic deletions seen in clinical reversion cases ranged from 1 to 1168 base pairs (in cDNA coordinates), with most deletions being <50 bp (FIG. 3E) and contained within a single exon. Therefore, while cells in culture appeared able to tolerate, for example, the loss of thousands of bases and multiple exons of BRCA2 coding sequence, this does not appear to be recapitulated clinically. This may reflect a greater requirement or fitness advantage for tumour cells with near-full length BRCA1 or BRCA2 proteins. It should be noted here that some NGS technologies or variant calling pipelines may not be optimised to detect large intragenic deletions or fusion events.

Interestingly, deletion size was generally larger in reversion mutations that displayed evidence of microhomology use, an observation that appeared to be limited to reversion mutations occurring in BRCA2-mutant tumours (BRCA1, p=0.60; BRCA2, p=0.0036; Wilcoxon rank sum test, FIG. 3E) perhaps reflecting a greater extent of end resection and microhomology search in BRCA2 mutant tumours than in BRCA1 mutant tumours. One reason for the increased deletion size in BRCA2 reversion mutations with microhomology could be that longer regions of microhomology are required for DNA end joining in this context. Longer regions of microhomology would be expected to occur less frequently, resulting in increased DNA resection length during microhomology searching. Consistent with this hypothesis, BRCA2 reversion mutations did indeed exhibit longer regions of microhomology on average, peaking at 2-3 nt, when compared with BRCA1 reversion events (which predominantly utilised 1 bp of microhomology on each side of the reversion deletion, FIG. 3F). A general consensus of opinion is that whilst canonical NHEJ exploits either no DNA sequence microhomology or very short regions (1-3 bp) to repair DNA, MMEJ and SSA exploit somewhat longer regions (2-20 bp and >15 bp, respectively (Bhargava et al., 2016; Sinha et al., 2016)). Taken at face value, this might therefore suggest that differences in DNA repair pathway usage could explain the differences in microhomology length associated with BRCA1 VS. BRCA2 reversion deletions. Understanding the common mutational outcomes can be used to predict likely reversion mutations for a given pathogenic mutation and thus refine designs of potential vaccines for prophylaxis.

### Example 7—Proximity of Reversion Mutations to Original Truncating Mutation Suggests that Many Revertant Proteins Will Constitute Neoantigens

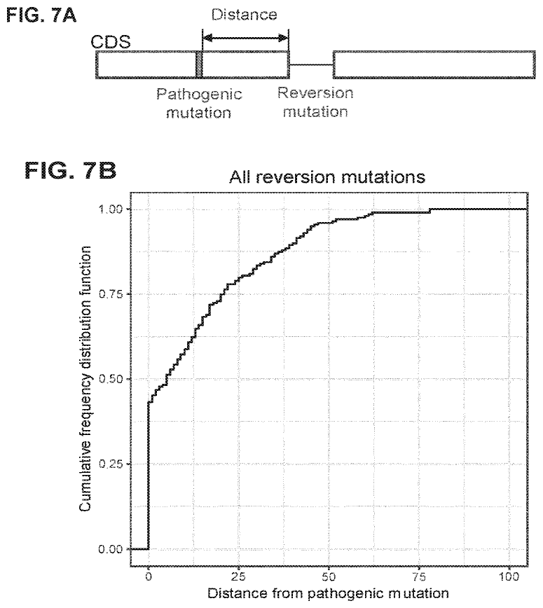

Compensatory frameshift reversions that do not restore the same codon as the original mutation (i.e. second site reversions) will introduce out-of-frame stretches of novel amino acid sequence in the revertant protein that are not encoded by the wild-type allele and may not be stably expressed from the pathogenic allele. Overall, 50% of reversions occurred at a distance of at least 6 bp from the pathogenic mutation, ranging up to 86 bp (FIG. 7). Thus, most revertant proteins will contain some out-of-frame sequence of 2-30 amino acids, or at least a novel breakpoint amino acid junction. These amino acid sequences may not have previously been visible to the host immune system and could constitute neoantigens. The inventors theorised that this could provide an opportunity to therapeutically target tumour cells expressing these candidate neoantigens, using approaches such as CAR-T cell therapies that target tumour cells expressing these neoantigens, immune checkpoint inhibitors or anticancer vaccines.

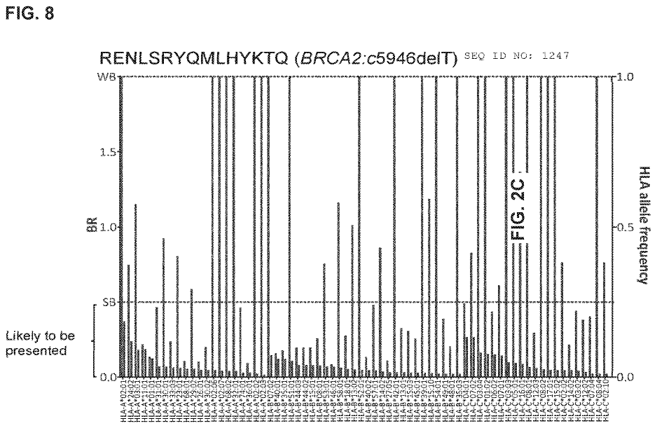

To assess this possibility, the inventors assessed how frequently reverted alleles contained out-of-frame amino acid sequences and whether peptides containing these out of frame sequences were likely to be presented by antigen-presenting HLA complexes. In the case of BRCA2:c.5946delT reversions, these contained out-of-frame peptide sequence ranging from 3-15 amino acids (FIG. 4A). Using the NetMHCpan 4.0 algorithm (Jurtz et al., 2017), the likelihood of antigen presentation of these peptides across a range of HLA allotypes was calculated. Peptides containing seven amino acids or longer (representing 3/10 revertant alleles analysed for this mutation) of the out-of-frame sequence following the c.5946delT mutation were predicted to be presented by the MHC in at least 75% of individuals (taking into account the population frequencies of different HLA types, see Materials and Methods) making them likely tumour antigens (FIG. 4B, FIG. 8). Similar frequencies of predicted neoantigen presentation frequency were calculated for the out-of-frame sequence following other pathogenic deletion mutations in the dataset, including other common founder mutations such as BRCA1:c.68_69delAG (66%, FIG. 4C). This out-of-frame sequence will be shared to some extent between reversions in patients with the same pathogenic mutation. Many of the actual neopeptides retained in the reverted alleles also had high predicted likelihoods of HLA presentation (FIG. 4D, Table 3). Without being bound by theory, the inventors believe that tumours with some revertant alleles may be targetable with immunotherapies that either relieve immune suppression or those that exploit the introduction of specific T cell clones that recognise specific neoepitopes. For some pathogenic mutations it may be possible to vaccinate against the peptides predicted to be expressed in revertant alleles prior to the commencement of PARPi or platinum therapy, as a route to delay or even prevent the emergence of therapy-resistant disease.

TABLE 3

| | | | % | |
|---|---|---|---|---|
| | | | individuals | |
| | | | predicted | |
| | | | to present | |
| | | | at least | |
| | Pathogenic | Reverted | one | |
| Gene | Mutation | allele | neopeptide | Annotation |
| BRCA1 | c.68_69delAG | c.68_69delAG; | 81 | BRCA1:c.68_69delAG; |
| | | 85delG | | 85delG |
| BRCA1 | c.68_69delAG | c.68_69delAG; | 66 | BRCA1:c.68_69delAG; |
| | | 113delA | | 113delA |
| BRCA1 | c.68_69delAG | c.68_69delAG; | 66 | BRCA1:c.68_69delAG; |
| | | 108delC | | 108delC |
| BRCA1 | c.68_69delAG | c.65_79delTAGAGTGTCCCATCT | 7 | BRCA1:c.65_79delTAGAGT |
| | | [SEQ ID NO: 1219] | | GTCCCATCT |
| | | | | [SEQ ID |
| | | | | NO: 1219] |
| BRCA1 | c.5266dupC | c.5272delA | 64 | BRCA1: |
| | | | | c.5266dupC; |
| | | | | 5272delA |
| BRCA1 | c.5266dupC | c.5266dupC; | 54 | BRCA1: |
| | | 5276_5277 | | c.5266dupC; |
| | | dupAG | | 5276_5277 |
| | | | | dupAG |
| BRCA2 | c.5946delT | c.5946delT; | 98 | BRCA2: |
| | | 5994_5999del | | c.5946delT; |
| | | AGTGTTinsTATC | | 5994_5999delAGTGTTinsTATC |
| BRCA2 | c.5946delT | c.5946delT; | 97 | BRCA2: |
| | | 5992_6005del | | c.5946delT; |
| | | CAAGTGTTTTCTGA | | 5992_6005delCAAGTGTT |
| | | [SEQ ID NO: | | TTCTGA |
| | | 1222] | | [SEQ ID |
| | | | | NO: 1222] |
| BRCA2 | c.5946delT | c.5946delT; | 96 | BRCA2: |
| | | 5998_6008del | | c.5946delT; |
| | | TTTTCTGAAAT | | 5998_6008delTTTTCTGA |
| | | [SEQ ID NO: | | AAT |
| | | 1220] insCAA | | [SEQ ID |
| | | | | NO: 1220] |
| | | | | insCAA |
| BRCA2 | c.5946delT | c.5946delT; | 84 | BRCA2: |
| | | 5964_5998 | | c.5946delT; |
| | | del35 | | 5964_5998del35 |
| BRCA2 | c.5946delT | c.5946_5990 | 75 | BRCA2: |
| | | del45 | | c.5946_5990del45 |
| BRCA2 | c.5946delT | c.5946delT; | 69 | BRCA2: |
| | | 5959_5966del | | c.5946delT; |
| | | CAGGTATC | | 5959_5966delCAGGTATC |
| BRCA2 | c.5946delT | c.5946delT; | 69 | BRCA2: |
| | | 5952_5959del | | c.5946delT; |
| | | ATCTGTCC | | 5952_5959delATCTGTCC |
| BRCA2 | c.5946delT | c.5946delT; | 59 | BRCA2: |
| | | 5949_5952 | | c.5946delT; |
| | | dupAAAA | | 5949_5952dupAAAA |
| BRCA2 | c.5946delT | c.5946delT; | 29 | BRCA2: |
| | | 5954_6090de | | c.5946delT; |
| | | ll37 | | 5954_6090del137 |
| BRCA2 | c.5946delT | c.5944_5952 | 25 | BRCA2: |
| | | delAGTGGAAAA | | c.5944_5952delAGTGGAAAA |
| BRCA2 | c.5946delT | c.5941_5956 | 4 | BRCA2: |
| | | delGCAAGTG | | c.5941_5956delGCAAGTGGA |
| | | GAAAATCTG | | AAATCTG |
| | | [SEQ ID NO: | | [SEQ ID |
| | | 1221] insA | | NO: 1221] |
| | | | | insA |

TABLE 4
Studies from cBioPortal used for analysis of pathogenic mutations
Study

Ovarian Serous Cystadenocarcinoma (TCGA, Provisional)
Breast Cancer (METABRIC, Nature 2012 & Nat Commun 2016)
Breast Invasive Carcinoma (TCGA, Cell 2015)
Breast Invasive Carcinoma (Sanger, Nature 2012)
Breast Cancer (MSK, Cancer Cell 2018)
The Metastatic Breast Cancer Project (Provisional,
October 2018)
Breast Cancer (MSKCC, 2019)
Metastatic Prostate Adenocarcinoma (SU2C/PCF Dream Team,
PNAS 2019)
Pancreatic Adenocarcinoma (QCMG, Nature 2016)
Breast Invasive Carcinoma (Broad, Nature 2012)
Breast Invasive Carcinoma (British Columbia, Nature 2012)
Metastatic Breast Cancer (INSERM, PLoS Med 2016)
Prostate Adenocarcinoma (MSKCC/DFCI, Nature Genetics 2018)
Pancreatic Adenocarcinoma (TCGA, Provisional)

TABLE 5
Selected Primary Neopeptides Retained in Observed Reversion Mutations

| SEQ | | | | Number |
|---|---|---|---|---|
| ID | | Individuals | | of |
| NO: | Neopeptide | presenting | Mutations | mutations |
| 1 | ILVSHLSGV | 577 | [‘c.68_69delAG\|c.65_79delTAGAGTGTCCCATCT | 4 |
| | | | [SEQ ID NO: 1219]’, | |
| | | | ‘c.68_69delAG\|c.85delG’, | |
| | | | ‘c.68_69delAG\|c.113delA’, | |
| | | | ‘c.68_69delAG\|c.108delC’] | |
| 2 | GVDQGTCLHK | 230 | [‘c.68_69delAG\|c.65_79delTAGAGTGTCCCATCT | 4 |
| | | | [SEQ ID NO: 1219]’, | |
| | | | ‘c.68_69delAG\|c.85delG’, | |
| | | | ‘c.68_69delAG\|c.113delA’, | |
| | | | ‘c.68_69delAG\|c.108delC’] | |
| 3 | NAMQKILV | 222 | [‘c.68_69delAG\|c.65_79delTAGAGTGTCCCATCT | 4 |
| | | | [SEQ ID NO: 1219]’, | |
| | | | ‘c.68_69delAG\|c.85delG’, | |
| | | | ‘c.68_69delAG\|c.113delA’, | |
| | | | ‘c.68_69delAG\|c.108delC’] | |
| 4 | MQKILVSHL | 28 | [‘.68_69delAG\|c.65_79delTAGAGTGTCCCATCT | 4 |
| | | | [SEQ ID NO: 1219]’, | |
| | | | ‘c.68_69delAG\|c.85delG’, | |
| | | | ‘c.68_69delAG\|c.113delA’, | |
| | | | ‘c.68_69delAG\|c.108delC’] | |
| 5 | KILVSHLSGV | 26 | [‘c.68_69delAG\|c.65_79delTAGAGTGTCCCATCT | 4 |
| | | | [SEQ ID NO: 1219]’, | |
| | | | ‘c.68_69delAG\|c.85delG’, | |
| | | | ‘c.68 69delAG\|c.113delA’, | |
| | | | ‘c.68_69delAG\|c.108delC’] | |
| 6 | ARNLLLWAL | 731 | [‘c.5266dupC\|c.5276 5277dupAG’, | 2 |
| | | | ‘c.5266dupC\|c.5272delA’] | |
| 7 | RESPGQKDL | 411 | [‘c.5266dupC\|c.5276 5277dupAG’, | 2 |
| | | | ‘c.5266dupC\|c.5272delA’] | |
| 8 | STGMDGTAVW | 280 | [‘c.5266dupC\|c.5276 5277dupAG’, | 2 |
| | | | ‘c.5266dupC\|c.5272delA’] | |
| 9 | TGMDGTAVW | 250 | [‘c.5266dupC\|c.5276 5277dupAG’, | 2 |
| | | | ‘c.5266dupC\|c.5272delA’] | |
| 10 | AFIIHPWHR | 217 | [‘c.5266dupC\|c.5276 5277dupAG’, | 2 |
| | | | ‘c.5266dupC\|c.5272delA’] | |
| 11 | GEGAFIIHPW | 195 | [‘c.5266dupC\|c.5276 5277dupAG’, | 2 |
| | | | ‘c.5266dupC\|c.5272delA’] | |
| 12 | RSTGMDGTAVW | 194 | [‘c.5266dupC\|c.5276 5277dupAG’, | 2 |
| | | | ‘c.5266dupC\|c.5272delA’] | |
| 13 | QGARNLLLW | 178 | [‘c.5266dupC\|c.5276 5277dupAG’, | 2 |
| | | | ‘c.5266dupC\|c.5272delA’] | |
| 14 | QHAHRSTGM | 119 | [‘c.5266dupC\|c.5276 5277dupAG’, | 2 |
| | | | ‘c.5266dupC\|c.5272delA’] | |
| 15 | GAFIIHPWHR | 109 | [‘c.5266dupC\|c.5276_5277dupAG’, | 2 |
| | | | ‘c.5266dupC\|c.5272delA’] | |
| 16 | FIIHPWHR | 97 | [‘c.5266dupC\|c.5276_5277dupAG’, | 2 |
| | | | ‘c.5266dupC\|c.5272delA’] | |
| 17 | WALHQHAHR | 97 | [‘c.5266dupC\|c.5276_5277dupAG’, | 2 |
| | | | ‘c.5266dupC\|c.5272delA’] | |
| 18 | HAHRSTGM | 94 | [‘c.5266dupC\|c.5276_5277dupAG’, | 2 |
| | | | ‘c.5266dupC\|c.5272delA’] | |
| 19 | KRARESPGQK | 86 | [‘c.5266dupC\|c.5276_5277dupAG’, | 2 |
| | | | ‘c.5266dupC\|c.5272delA’] | |
| 20 | RARESPGQK | 86 | [‘c.5266dupC\|c.5276_5277dupAG’, | 2 |
| | | | ‘c.5266dupC\|c.5272delA’] | |
| 21 | EGAFIIHPW | 83 | [‘c.5266dupC\|c.5276_5277dupAG’, | 2 |
| | | | ‘c.5266dupC\|c.5272delA’] | |
| 22 | AARCLDRGQW | 56 | [‘c.5266dupC\|c.5276_5277dupAG’, | 2 |
| | | | ‘c.5266dupC\|c.5272delA’] | |
| 23 | GARNLLLW | 56 | [‘c.5266dupC\|c.5276_5277dupAG’, | 2 |
| | | | ‘c.5266dupC\|c.5272delA’] | |
| 24 | ARCLDRGQWL | 43 | [‘c.5266dupC\|c.5276_5277dupAG’, | 2 |
| | | | ‘c.5266dupC\|c.5272delA’] | |
| 25 | GEGAFIIHP | 32 | [‘c.5266dupC\|c.5276_5277dupAG’, | 2 |
| | | | ‘c.5266dupC\|c.5272delA’] | |
| 26 | EGAFIIHPWHR | 18 | [‘c.5266dupC\|c.5276_5277dupAG’, | 2 |
| | | | ‘c.5266dupC\|c.5272delA’] | |
| 27 | KDLQGARNL | 15 | [‘c.5266dupC\|c.5276_5277dupAG’, | 2 |
| | | | ‘c.5266dupC\|c.5272delA’] | |
| 28 | GARNLLLWAL | 1 | [‘c.5266dupC\|c.5276_5277dupAG’, | 2 |
| | | | ‘c.5266dupC\|c.5272delA’] | |
| 29 | LQGARNLLL | 1 | [‘c.5266dupC\|c.5276_5277dupAG’, | 2 |
| | | | ‘c.5266dupC\|c.5272delA’] | |
| 30 | TARENLSRY | 672 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964 5998del35’] | |
| 31 | RENLSRYQM | 630 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 32 | KTQDKCFLK | 604 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 33 | STARENLSR | 507 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 34 | IFSTARENL | 416 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 35 | STARENLSRY | 384 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 36 | SRYQMLHY | 199 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 37 | NTCGIFSTAR | 162 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 38 | ARENLSRYQM | 100 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 39 | LSRYQMLHY | 99 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 40 | ENLSRYQML | 94 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 41 | NLSRYQML | 94 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 42 | FSTARENLSR | 67 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 43 | SRYQMLHYK | 48 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 44 | RENLSRYQML | 14 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |
| 45 | NLSRYQMLHY | 6 | [‘c.5946delT\|c.5998_6008delTTTTCTGAAAT | 11 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5941_5956 | |
| | | | delGCAAGTGGAAAATCTG | |
| | | | [SEQ ID NO: 1221]insA’, | |
| | | | ‘c.5946delT\|c.5944_5952delAGTGGAAAA’, | |
| | | | ‘c.5946delT\|c.5952_5959delATCTGTCC’, | |
| | | | ‘c.5946delT\|c.5992_6005 | |
| | | | delCAAGTGTTTTCTGA[SEQ ID NO: 1222]’, | |
| | | | ‘c.5946delT\|c.5964_5998del35’] | |

TABLE 6
Selected Reversion Mutations Encoding Neopeptides

| SEQ | | | | Number |
|---|---|---|---|---|
| ID | | Individuals | | of |
| NO: | Neopeptide | presenting | Mutations | mutations |
| 4 | MQKILVSHL | 28 | [‘c.68_69delAG\|c.85delG’, | 3 |
| | | | ‘c.68_69delAG\|c.113delA’, | |
| | | | ‘c.68_69delAG\|c.108delC’] | |
| 3 | NAMQKILV | 222 | [‘c.68_69delAG\|c.85delG’, | 3 |
| | | | ‘c.68_69delAG\|c.113delA’, | |
| | | | ‘c.68_69delAG\|c.108delC’] | |
| 1 | ILVSHLSGV | 577 | [‘c.68_69delAG\|c.113delA’, | 2 |
| | | | ‘c.68_69delAG\|c.108delC’] | |
| 5 | KILVSHLSGV | 26 | [‘c.68_69delAG\|c.113delA’, | 2 |
| | | | ‘c.68_69delAG\|c.108delC’] | |
| 2 | GVDQGTCLHK | 230 | [‘c.68_69delAG\|c.113delA’] | 1 |
| 46 | GVDQGTCLTK | 230 | [‘c.68_69delAG\|c.108delC’] | 1 |
| 47 | ILVSHLSEL | 838 | [‘c.68_69delAG\|c.85delG’] | 1 |
| 48 | NAMQKICL | 94 | [‘c.68_69delAG\|c.65_79del | 1 |
| | | | TAGAGTGTCCCATCT[SEQ ID NO: 1219]’ | |
| 49 | SELIKEPVSTK | 215 | [‘c.68_69delAG\|c.85delG’] | 1 |
| 50 | ARESPGQKEI | 103 | [‘c.5266dupC\|c.5276_5277dupAG’] | 1 |
| 51 | ARESPGRKIF | 200 | [‘c.5266dupC\|c.5272delA’] | 1 |
| 52 | ESPGRKIFR | 97 | [‘c.5266dupC\|c.5272delA’] | 1 |
| 53 | GQKEIFRGL | 82 | [‘c.5266dupC\|c.5276_5277dupAG’] | 1 |
| 54 | GRKIFRGLEI | 5 | [‘c.5266dupC\|c.5272delA’] | 1 |
| 55 | KEIFRGLEI | 639 | [‘c.5266dupC\|c.5276_5277dupAG’] | 1 |
| 19 | KRARESPGQK | 86 | [‘c.5266dupC\|c.5276_5277dupAG’] | 1 |
| 20 | RARESPGQK | 86 | [‘c.5266dupC\|c.5276_5277dupAG’] | 1 |
| 56 | RARESPGRK | 86 | [‘c.5266dupC\|c.5272delA’] | 1 |
| 57 | RESPGQKEI | 589 | [‘c.5266dupC\|c.5276_5277dupAG’] | 1 |
| 58 | RESPGQKEIF | 336 | [‘c.5266dupC\|c.5276_5277dupAG’] | 1 |
| 59 | RESPGRKI | 26 | [‘c.5266dupC\|c.5272delA’] | 1 |
| 60 | RESPGRKIF | 558 | ['c.5266dupC\|c.5272delA’] | 1 |
| 61 | SPGRKIFRGL | 217 | [‘c.5266dupC\|c.5272delA’] | 1 |
| 37 | NTCGIFSTAR | 162 | [‘c.5946delT\|c.5998_6008delTTTTCTGAAAT | 9 |
| | | | [SEQ ID NO: 1220]insCAA’, | |
| | | | ‘c.5946delT\|c.5949_5952dupAAAA’, | |
| | | | ‘c.5946delT\|c.5994_5999delAGTGTTinsTATC’, | |
| | | | ‘c.5946delT\|c.5946_5990del45’, | |
| | | | ‘c.5946delT\|c.5954_6090del137’, | |
| | | | ‘c.5946delT\|c.5959_5966delCAGGTATC’, | |
| | | | ‘c.5946delT\|c.5952_5959delATCTGTCC’, | |
| | | | ‘c.5946delT\|c.5992_6005delCAAGTGTTTTCTGA | |
| | | | [SEQ ID NO: 1222]’, | |
| | | | ‘c.5946delT\|c.5964_5998del35’] | |
| 34 | IFSTARENL | 416 | [‘c.5946delT\|c.5998_6008delTTTTCTGAAAT | 5 |
| | | | [SEQ ID NO: 1220]insCAA’, | |
| | | | ‘c.5946delT\|c.5994_5999delAGTGTTinsTATC’, | |
| | | | ‘c.5946delT\|c.5959_5966delCAGGTATC’, | |
| | | | ‘c.5946delT\|c.5992_6005delCAAGTGTTTTCTGA | |
| | | | [SEQ ID NO: 1222]’, | |
| | | | ‘c.5946delT\|c.5964_5998del35’] | |
| 42 | FSTARENLSR | 67 | [‘c.5946delT\|c.5998_6008delTTTTCTGAAAT | 4 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID: 1222]’, | |
| | | | ‘c.5946delT\|c.5964_5998del35’] | |
| 33 | STARENLSR | 507 | [‘c.5946delT\|c.5998_6008delTTTTCTGAAAT | 4 |
| | | | [SEQ ID: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’, | |
| | | | ‘c.5946delT\|c.5964_5998del35’] | |
| 38 | ARENLSRYQM | 100 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 40 | ENLSRYQML | 94 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 39 | LSRYQMLHY | 99 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 41 | NLSRYQML | 94 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 45 | NLSRYQMLHY | 6 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 31 | RENLSRYQM | 630 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 44 | RENLSRYQML | 14 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 36 | SRYQMLHY | 199 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 43 | SRYQMLHYK | 48 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 35 | STARENLSRY | 384 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 30 | TARENLSRY | 672 | [‘c.5946delT\|c.5998_6008delTTTTCTGAAAT | 3 |
| | | | [SEQ ID NO: 1220]insCAA’, | |
| | | | ‘c.5946delT\|c.5994_5999delAGTGTTinsTATC’, | |
| | | | ‘c.5946delT\|c.5992_6005delCAAGTGTTTTCTGA | |
| | | | [SEQ ID NO: 1222]’] | |
| 62 | GIFSTAREK | 550 | [‘c.5946delT\|c.5949_5952dupAAAA’, | 2 |
| | | | ‘c.5946delT\|c.5952_5959delATCTGTCC’] | |
| 63 | AREKVSDASL | 2 | [‘c.5946delT\|c.5952_5959delATCTGTCC’] | 1 |
| 64 | ARENLSDASL | 189 | [‘c.5946delT\|c.5959_5966delCAGGTATC’] | 1 |
| 65 | ENLSRFSEI | 94 | [‘c.5946delT\|c.5964_5998del35’] | 1 |
| 66 | GIFSTAREKK | 216 | [‘c.5946delT\|c.5949_5952dupAAAA’] | 1 |
| 67 | IFSTARQVF | 802 | [‘c.5946delT\|c.5946_5990del45’] | 1 |
| 68 | IFSTASVQV | 312 | [‘c.5946delT\|c.5944_5952delAGTGGAAAA’] | 1 |
| 69 | KEDSTKQVF | 596 | [‘c.5946delT\|c.5998_6008delTTTTCTGAAAT | 1 |
| | | | [SEQ ID NO: 1220]insCAA’] | |
| 70 | KTQDISSEI | 600 | [‘c.5946delT\|c.5994_5999delAGTGTTinsTATC'] | 1 |
| 71 | MLHYKTQDI | 94 | [‘c.5946delT\|c.5994_5999delAGTGTTinsTATC'] | 1 |
| 72 | MLHYKTQEI | 192 | [‘c.5946delT\|c.5992_6005delCAAGTGTTTTCTGA | 1 |
| | | | [SEQ ID NO: 1222]’] | |
| 73 | NTCGIFSTI | 54 | [‘c.5946delT\|c.5941_5956delGCAAGTGGAAAATCTG | 1 |
| | | | [SEQ ID NO: 1221]GinsA’] | |
| 74 | QEIEDSTKQV | 262 | [‘c.5946delT\|c.5992_6005delCAAGTGTTTTCTGA | 1 |
| | | | [SEQ ID NO: 1222]’] | |
| 75 | QEIEDSTKQVF | 255 | [‘c.5946delT\|c.5992_6005delCAAGTGTTTTCTGA | 1 |
| | | | [SEQ ID NO: 1222]’] | |
| 76 | REKKSVQVS | 18 | [‘c.5946delT\|c.5949_5952dupAAAA’] | 1 |
| 77 | REKVSDASL | 318 | [‘c.5946deIT\|c.5952_5959delATCTGTCC’] | 1 |
| 78 | RENLSDASL | 552 | [‘c.5946deIT\|c.5959_5966delCAGGTATC’] | 1 |
| 79 | RENTAIRTP | 76 | [‘c.5946delT\|c.5954_6090del137’] | 1 |
| 80 | SRFSEIEDSTK | 43 | [‘c.5946delT\|c.5964_5998del35’] | 1 |
| 81 | STARENLSRF | 179 | [‘c.5946delT\|c.5964_5998del35’] | 1 |
| 82 | STARENTAI | 52 | [‘c.5946delT\|c.5954_6090del137’] | 1 |
| 83 | STARENTAIR | 77 | [‘c.5946delT\|c.5954_6090del137’] | 1 |
| 84 | TAREKKSV | 94 | [‘c.5946delT\|c.5949_5952dupAAAA’] | 1 |
| 85 | TAREKVSDASL | 94 | [‘c.5946deIT\|c.5952_5959delATCTGTCC’] | 1 |
| 86 | TARENLSRF | 665 | [‘c.5946delT\|c.5964_5998del35’] | 1 |
| 87 | TARENTAI | 94 | [‘c.5946delT\|c.5954_6090del137’] | 1 |
| 88 | TARENTAIR | 170 | [‘c.5946delT\|c.5954_6090del137’] | 1 |
| 89 | TARQVFSEI | 240 | [‘c.5946delT\|c.5946_5990del45’] | 1 |

TABLE 7
Presentation scores

| | | | HLA |
|---|---|---|---|
| | Pathogenic | | presentation |
| Gene | mutation | Reversion mutation | likelihood |
| BRCA1 | c.68_69delAG | c.85delG | 81 |
| BRCA1 | c.68_69delAG | c.108delC | 66 |
| BRCA1 | c.68_69delAG | c.113delA | 66 |
| BRCA1 | c.68_69delAG | c.65_79delTAGAGTGTCCCAT | 7 |
| | | CT [SEQ ID NO: 1219] | |
| BRCA1 | c.5266dupC | c.5272delA | 64 |
| BRCA1 | c.5266dupC | c.5276_5277dupAG | 54 |
| BRCA2 | c.5946delT | c.5994_5999delAGTGTTinsTATC | 98 |
| BRCA2 | c.5946delT | c.5992_6005delCAAGTGTTT | 97 |
| | | TCTGA [SEQ ID NO: 1222] | |
| BRCA2 | c.5946delT | c.5998_6008delTTTTCTGAA | 96 |
| | | AT [SEQ ID NO: 1220] | |
| | | insCAA | |
| BRCA2 | c.5946delT | c.5964_5998del35 | 84 |
| BRCA2 | c.5946delT | c.5946_5990del45 | 75 |
| BRCA2 | c.5946delT | c.5952_5959delATCTGTCC | 69 |
| BRCA2 | c.5946delT | c.5959_5966delCAGGTATC | 69 |
| BRCA2 | c.5946delT | c.5949_5952dupAAAA | 59 |
| BRCA2 | c.5946delT | c.5954_6090del137 | 29 |
| BRCA2 | c.5946delT | c.5944_5952delAGTGGAAAA | 25 |
| BRCA2 | c.5946delT | c.5941_5956delGCAAGTGGA | 4 |
| | | AAATCTG [SEQ ID NO: 1221] | |
| | | insA | |

TABLE 8
Predicted binding level (WB-weak binder, % Rank < 2; SB-strong
binder, % Rank < 0.5) of the specified mutations

| | SEQ ID | | % | |
|---|---|---|---|---|
| MHC | NO: | Identity | Rank_EL | BindLevel |
| HLA-B*0801 | 3 | B1_185delAG_dow | 0.458 | SB |
| HLA-B*1501 | 4 | B1_185delAG_dow | 0.3 | SB |
| HLA-A*0201 | 1 | B1_185delAG_dow | 0.072 | SB |
| HLA-B*3801 | 1157 | B1_185delAG_dow | 0.224 | SB |
| HLA-B*0801 | 1158 | B1_185delAG_dow | 1.342 | WB |
| HLA-B*0801 | 1159 | B1_185delAG_dow | 0.683 | WB |
| HLA-A*0301 | 1160 | B1_185delAG_dow | 0.6 | WB |
| HLA-A*0201 | 1161 | B1_185delAG_dow | 1.687 | WB |
| HLA-B*1501 | 1162 | B1_185delAG_dow | 1.712 | WB |
| HLA-A*0201 | 5 | B1_185delAG_dow | 0.942 | WB |
| HLA-A*0101 | 1163 | B1_185delAG_dow | 1.313 | WB |
| HLA-A*0101 | 1164 | B1_185delAG_dow | 1.478 | WB |
| HLA-A*0101 | 1165 | B1_185delAG_dow | 0.987 | WB |
| HLA-A*0301 | 2 | B1_185delAG_dow | 0.676 | WB |
| HLA-A*0301 | 20 | B1_5382insC_dow | 0.411 | SB |
| HLA-B*4001 | 1166 | B1_5382insC_dow | 0.483 | SB |
| HLA-B*4001 | 7 | B1_5382insC_dow | 0.068 | SB |
| HLA-B*5801 | 13 | B1_5382insC_dow | 0.098 | SB |
| HLA-B*2705 | 6 | B1_5382insC_dow | 0.033 | SB |
| HLA-B*3901 | 14 | B1_5382insC_dow | 0.189 | SB |
| HLA-B*5801 | 12 | B1_5382insC_dow | 0.021 | SB |
| HLA-B*5801 | 8 | B1_5382insC_dow | 0.1 | SB |
| HLA-B*5801 | 9 | B1_5382insC_dow | 0.253 | SB |
| HLA-B*0702 | 1167 | B1_5382insC_dow | 0.941 | WB |
| HLA-B*2705 | 19 | B1_5382insC_dow | 0.764 | WB |
| HLA-B*4001 | 1168 | B1_5382insC_dow | 0.688 | WB |
| HLA-B*1501 | 1169 | B1_5382insC_dow | 1.929 | WB |
| HLA-B*4001 | 27 | B1_5382insC_dow | 1.53 | WB |
| HLA-B*5701 | 1170 | B1_5382insC_dow | 1.566 | WB |
| HLA-B*0801 | 1171 | B1_5382insC_dow | 1.842 | WB |
| HLA-B*5701 | 1172 | B1_5382insC_dow | 0.553 | WB |
| HLA-B*2705 | 1173 | B1_5382insC_dow | 1.321 | WB |
| HLA-B*5701 | 23 | B1_5382insC_dow | 0.612 | WB |
| HLA-B*2705 | 28 | B1_5382insC_dow | 0.621 | WB |
| HLA-B*2705 | 1174 | B1_5382insC_dow | 0.893 | WB |
| HLA-B*2705 | 1175 | B1_5382insC_dow | 1.822 | WB |
| HLA-A*0201 | 1176 | B1_5382insC_dow | 0.72 | WB |
| HLA-B*1501 | 1177 | B1_5382insC_dow | 0.799 | WB |
| HLA-B*0801 | 18 | B1_5382insC_dow | 1.378 | WB |
| HLA-B*5801 | 1178 | B1_5382insC_dow | 0.83 | WB |
| HLA-B*5801 | 1179 | B1_5382insC_dow | 1.352 | WB |
| HLA-B*5801 | 1180 | B1_5382insC_dow | 1.465 | WB |
| HLA-B*5801 | 21 | B1_5382insC_dow | 0.773 | WB |
| HLA-B*5801 | 1181 | B1_5382insC_dow | 0.722 | WB |
| HLA-B*2705 | 1182 | B1_5382insC_dow | 1.764 | WB |
| HLA-B*5701 | 1183 | B1_5382insC_dow | 1.668 | WB |
| HLA-B*5701 | 22 | B1_5382insC_dow | 0.625 | WB |
| HLA-B*2705 | 1184 | B1_5382insC_dow | 1.407 | WB |
| HLA-B*2705 | 24 | B1_5382insC_dow | 0.775 | WB |
| HLA-B*5801 | 1185 | B1_5382insC_dow | 1.756 | WB |
| HLA-B*5801 | 1186 | B1_5382insC_dow | 0.669 | WB |
| HLA-B*5701 | 1187 | B1_5382insC_ups | 0.023 | SB |
| HLA-B*0801 | 1188 | B1_5382insC_ups | 0.13 | SB |
| HLA-B*5801 | 1189 | B1_5382insC_ups | 0.426 | SB |
| HLA-B*5801 | 1190 | B1_5382insC_ups | 0.058 | SB |
| HLA-B*4001 | 1191 | B1_5382insC_ups | 0.397 | SB |
| HLA-B*0801 | 1192 | B1_5382insC_ups | 1.481 | WB |
| HLA-B*5801 | 1193 | B1_5382insC_ups | 1.204 | WB |
| HLA-B*1501 | 1194 | B1_5382insC_ups | 1.767 | WB |
| HLA-B*3901 | 1195 | B1_5382insC_ups | 0.916 | WB |
| HLA-A*0201 | 1196 | B1_5382insC_ups | 1.68 | WB |
| HLA-A*0201 | 1197 | B1_5382insC_ups | 1.791 | WB |
| HLA-B*4001 | 1198 | B1_5382insC_ups | 0.97 | WB |
| HLA-B*4001 | 1199 | B1_5382insC_ups | 1.239 | WB |
| HLA-B*4001 | 1200 | B1_5382insC_ups | 1.289 | WB |
| HLA-B*0801 | 1201 | B1_5382insC_ups | 0.556 | WB |
| HLA-B*0801 | 1202 | B1_5382insC_ups | 0.883 | WB |
| HLA-A*0101 | 1203 | B2_6174delT_dow | 0.46 | SB |
| HLA-A*2601 | 35 | B2_6174delT_dow | 0.075 | SB |
| HLA-A*2601 | 30 | B2_6174delT_dow | 0.125 | SB |
| HLA-B*4001 | 31 | B2_6174delT_dow | 0.134 | SB |
| HLA-B*4001 | 44 | B2_6174delT_dow | 0.367 | SB |
| HLA-B*0801 | 40 | B2_6174delT_dow | 0.226 | SB |
| HLA-B*0801 | 41 | B2_6174delT_dow | 0.423 | SB |
| HLA-B*2705 | 36 | B2_6174delT_dow | 0.466 | SB |
| HLA-B*2705 | 43 | B2_6174delT_dow | 0.056 | SB |
| HLA-B*3801 | 1204 | B2_6174delT_dow | 0.24 | SB |
| HLA-B*3801 | 1205 | B2_6174delT_dow | 0.481 | SB |
| HLA-A*0301 | 32 | B2_6174delT_dow | 0.155 | SB |
| HLA-A*2402 | 34 | B2_6174delT_dow | 0.703 | WB |
| HLA-A*0301 | 33 | B2_6174delT_dow | 1.35 | WB |
| HLA-A*0101 | 1206 | B2_6174delT_dow | 1.619 | WB |
| HLA-B*2705 | 38 | B2_6174delT_dow | 0.821 | WB |
| HLA-B*2705 | 1207 | B2_6174delT_dow | 1.345 | WB |
| HLA-B*0801 | 1208 | B2_6174delT_dow | 0.936 | WB |
| HLA-A*0101 | 45 | B2_6174delT_dow | 0.905 | WB |
| HLA-B*2705 | 1209 | B2_6174delT_dow | 1.057 | WB |
| HLA-A*0101 | 39 | B2_6174delT_dow | 0.531 | WB |
| HLA-B*2705 | 1210 | B2_6174delT_dow | 0.822 | WB |
| HLA-B*2705 | 1211 | B2_6174delT_dow | 1.14 | WB |
| HLA-B*2705 | 1212 | B2_6174delT_dow | 1.784 | WB |
| HLA-A*0301 | 1213 | B2_6174delT_dow | 1.23 | WB |
| HLA-A*0301 | 1214 | B2_6174delT_dow | 0.67 | WB |
| HLA-B*3901 | 1215 | B2_6174delT_dow | 0.999 | WB |
| HLA-A*2402 | 1216 | B2_6174delT_dow | 0.756 | WB |
| HLA-B*3901 | 1217 | B2_6174delT_dow | 1.299 | WB |
| HLA-B*0801 | 1218 | B2_6174delT_ups | 0.227 | SB |

The amino acid sequence of the neopeptides that result from the BRCA1 and BRCA2 reversion mutations set forth in Table 3 above may be determined from the reversion mutation, sequence of the BRCA1 or BRCA2 gene, the genomic sequences of which are available from the NCBI at the accession numbers disclosed above, respectively. The genetic code can then be used to determine the amino acid sequence of the reading frame following the relevant mutation described above.

For example, the BRCA2 muation c.5998_6008delTTTTCTGAAATinsCAA encodes the out-of-frame amino acid sequence, as shown boxed in FIG. 4B, row three.

### Discussion

The inventors have shown that by collating, codifying and analysing >200 HR-gene reversion mutations, a number of principles can be established. These include the unique nature of most reversions, positional “hotspots” and “deserts” in the N- and C-terminal coding regions of BRCA2, the paucity of missense and splice-site pathogenic mutations leading to reversions, and differences in microhomology use in BRCA1 compared to BRCA2-related reversions. Finally, it was found that many reverted alleles were predicted to encode highly immunogenic neo-peptides, suggesting a route to treatment of reverted disease. The inventors believe that by generating, analysing and expanding the reversion dataset, additional principles that govern how therapy resistance emerges in HR-defective cancers could be established.

The inventors noted that the clinical reversion mutations seem to have a more restricted spectrum (<100 bp deletions, close to the pathogenic mutation (FIG. 2A, FIG. 3E, FIG. 7)) compared to those previously seen in cell line and PDX studies, where large deletions predominate (Edwards et al., 2008; Sakai et al., 2008; Ter Brugge et al., 2016). Although some ascertainment bias in the detection of clinical reversions cannot be eliminated, it seems that the types of reversions seen in patients are more likely to preserve the majority of the coding sequence than those seen in preclinical models. Furthermore, in contrast to the ubiquitous microhomology at deletions in cell line studies, the inventors found that microhomology usage in clinical reversions was not universal (66% of the deletion-mediated reversion mutations exhibiting microhomology). Without being bound by theory, the inventors believe that multiple DNA repair processes might drive reversion, implying that the design of therapeutic interventions that limit reversions might be more complex than originally thought. Tumour sequencing studies have assessed microhomology usage in somatic deletion mutations at a genome-wide level, finding, for example, that ≈40% of deletions (IQR, 30-50) showed microhomology in BRCA1/2 mutant breast cancers, compared to ≈20% in BRCA wild-type (Davies et al., 2017)). Thus, the frequency of microhomology-associated BRCA-gene reversions is at the upper end of what might be expected at the genome-wide level in BRCA-gene mutant cancers, but still lower than that seen for reversions isolated from cell line models. Interestingly very few non-microhomology-mediated reversions in breast cancer cases (FIG. 3D) were observed, but this may be due to the relatively low numbers of patients reported.

The observation of a possible hotspot for secondary mutations around position c.750-775 in BRCA2 has potential implications for patients with these mutations. This may indicate that patients with such mutations would be at higher risk of acquiring resistance via reversions mutations, and should be monitored more closely. Conversely, patients with missense and splice site mutations, or mutations in the BRCA2 C-terminal desert (position c.7500 onwards) may be at lower risk of developing resistance via reversion.

As more is understood about the prevalence and nature of reversion mutations, the question of how to treat cancers that acquire drug resistance via secondary mutation can be addressed. After performing this analysis and without being bound by theory, the inventors suggest several possibilities. First, as described above, inhibiting microhomology-mediated end joining may be a way of preventing the emergence of some reversions, although this might not be a completely effective approach, given the frequency of non-microhomology mediated events observed. Targeting the reverted protein in some way may also be possible where this differs from wild type; for example, the mutant proteins may have an increased dependence on chaperones such as heat shock proteins to fold correctly. Where inserted or out-of-frame amino acid sequences are formed by reversion, these may be immunogenic. The inventors have demonstrated that there is a high probability of presentation by the MHC for many of the revertant sequences, including at common founders such as BRCA2:c.5946delT (FIG. 4). Thus, immunotherapies or cancer vaccines may also be an option for direct targeting of the revertant protein. There are other possible approaches that are not related to the revertant protein per se, such as using WEE1 or ATR inhibitors, that have been empirically shown in pre-clinical models to target BRCA-gene mutant tumour cells even after the acquisition of reversion mutations (Dréan et al., 2017), an effect likely mediated by the general replication stress that is likely to still exist in the tumour, despite reversion.

The analysis of all published clinically-occurring reversion events (reversions.icr.ac.uk) indicates that 66% of BRCA1 and 85% of BRCA2 reversion events are of these latter two classes and are mediated by deletions, which in most cases result in a new protein sequence (neopeptide) being encoded as shown in FIG. 4A. Almost all reversion mutations, with the exception of true reversions to wild type, will encode at least one novel amino acid or junction sequence. Using an in silico prediction of how likely these neopeptides are to be presented as antigens by MHC complexes (Punta et al. 2019; Jurtz et al. 2017), the inventors found that for most deletion-mediated reversion events the resulting neopeptides were highly likely to be presented by the MHC, taking population HLA frequencies into account including at common founder mutations such as BRCA2:c.6174delT (FIG. 4B). Across all reversions seen in clinical cases of PARPi or platinum resistance, the inventors found that the median percentage of individuals predicted to present at least one peptide for a reversion mutation was 35% (FIG. 4D). In most cases of reversions the HLA type of the individual they arose was not published, so a more precise estimate cannot be made. However for some specific, and indeed common, BRCA-gene pathogenic mutations, the likelihood of reversion neoantigens being presented by the MHC was even higher. For example, for reversions derived from individuals with pathogenic BRCA2:c.6174delT mutations, up to 918 of individuals were predicted to present neopeptides from the published cases of reversion mutation (FIG. 4B).

### Vaccination as a Strategy to Prevent or Treat Drug Resistance Caused by BRCA Reversions

Without being bound by theory, the analysis of all published cases of reversion mutations suggested that this novel out of frame sequence often constitutes a potential tumour neoantigen, with a high predicted probability of antigen presentation by the MHC, opening the possibility that PARP inhibitor resistant cancers could be targeted by exploiting the presentation of a BRCA reversion neoantigen. This could be via an anticancer vaccine or immune checkpoint inhibition. The inventors propose to test this theory using: (1) human T-cell priming assays using predicted neoantigens derived from revertant BRCA proteins; (2) the generation of revertant syngeneic mouse tumour models and a matched anticancer vaccine to test whether vaccinated mice reject BRCA-reverted tumours; and (3) treatment of mice bearing established syngeneic reverted tumours with the vaccine and immune checkpoint inhibitors to assess whether this may represent a viable therapeutic strategy to targeting this area of unmet clinical need.

Below, a strategy is described to test the theory that reversion mutations could be targeted by exploiting the formation of BRCA1 or BRCA2 neoepitopes that form when reversion mutations occur. This may provide the pre-clinical rationale for developing novel therapeutics which target cancers in the growing population of people who display PARPi or platinum salt resistant disease caused by reversion mutations.

Without being bound by theory, the inventors believe that if the immune system could be primed to target tumour cells presenting potentially antigenic neopeptides caused by reversion mutations, this may reduce or delay the emergence of PARPi or platinum salt resistance in individuals with pathogenic BRCA-gene mutations. This could be accomplished by using an anticancer vaccine that targets cells presenting antigenic neopeptides, for example, by using peptide, RNA or oncolytic virus-based vaccination. If the reverted BRCA-protein sequences are indeed immunogenic, this may also argue in favour of the use of immune checkpoint inhibitors to further boost the anti-neoantigen T cell response, in patients likely to have antigenic reversions.

These predictions of immunogenicity may be tested experimentally, through validation of the computational predictions described above and the development and use of syngeneic mouse tumour cell line models of drug resistance caused by reversion mutations. If successful, these experiments could form the basis for the clinical development of reversion vaccines. These could take the form of both personalised approaches for rare pathogenic mutations or “off-the-shelf” vaccines that would work in patients with more common founder mutations in either BRCA1 or BRCA2.

### Example 8—T-Cell Priming Assays: Are Predicted Reversion Neopeptides Presented by the MHC?

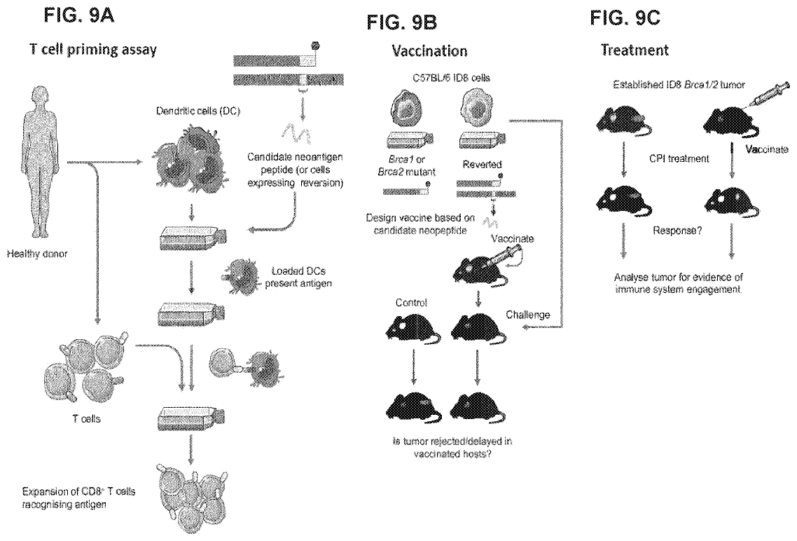

To assess whether the neopeptides with a high predicted likelihood of MHC presentation are able to be recognised by T-cells, T-cell priming assays will be carried out (FIG. 9A). This assay will be carried out using neoantigens from a number of sources. First, 5-10 peptides for the most highly ranked predictions for candidate neoantigens observed in clinical cases of BRCA-gene reversion will be used. Healthy donor-derived dendritic cells (DC)—which are specialised antigen presenting cells—will be pulsed with synthetic peptides corresponding to the sequences to be tested. Several model systems in the laboratory where pairs of cells with reversions have been matched with their parental cells carrying the pathogenic mutation only. Two of these are cell line models with CRISPR-Cas9 engineered reversion mutations (Dréan et al. 2017) and two are PDX tumours with spontaneously arising reversions (Tutt, Lord, Pettitt et al., unpublished). Cells from these models will be used directly to load DCs from donors, including those with matched HLA types.

These loaded DCs will be mixed with matched donor T-cells. If the dendritic cells are able to prime a T-cell response against the neoantigen, T-cells with T-cell receptors that recognise the antigen will proliferate and expand in number. This can be assessed by analysis of expansion of responder CD8+ cells by FACS or for intracellular IFN-g by ELISPOT. These peptide priming assays can be based on long or shorter, HLA-restricted, peptides as previously published (Prestwich et al. 2008; Jennings et al. 2019). As a complementary approach, the MHC-bound peptides presented in these models will also be profiled directly by mass spectrometry.

### Example 9—Anticancer Vaccination: Do Reversion Mutations Result in Tumour Rejection?

Several mouse tumour cell lines from inbred strains exist which can be transplanted back into hosts of the same strain as syngeneic tumour grafts. One of these—ID8, derived from an ovarian tumour—has been modified by CRISPR-Cas9 mutagenesis to make sublines with both Trp53 and Brca1 or Brca2 frameshift mutations, which closely resemble typical human BRCA1 and BRCA2 pathogenic mutations (Walton et al. 2017; Walton et al. 2016). These cells can be transplanted into C57BL/6 host mice and grown as syngeneic tumours, providing a useful model for BRCA1/2 deficient cancer. In theory these mutations should be revertible, providing an opportunity to study reversions in the context of a functional immune system. The neopeptides associated with the engineered frameshift mutations are predicted to bind the C57BL/6 H2b MHC complex.

Reversions in ID8-Brca mutant cells will be generated using an in vitro CRISPR-Cas9 mutagenesis approach that exploits guide RNAs that cause DNA breaks downstream of the pathogenic Brca-gene mutation. The inventors have previously employed this approach to generate reversions in either Capan-1 (BRCA2 mutant) or SUM149 (BRCA1 mutant) human tumour cells (Dréan et al. 2017). After verifying that the reversions retain predicted neoantigens, these tumours will be transplanted into C57BL/6 hosts with or without prior vaccination with a neoantigen epitope (FIG. 9B). If the tumour graft is rejected after vaccination, this would support the idea that vaccination at the outset of treatment could be used as a strategy to prevent establishment of revertant tumour clones. It is also possible that reverted cells will be rejected even without vaccination, as they may be more inherently immunogenic, which would in itself be informative in terms of understanding which reversion events might or might not be tolerated/recognised by the immune system. Vaccines will be mouse dendritic cell (DC) based, generated via loading of purified syngeneic DC with the predicted neopeptide, as this is likely to result in optimal antigen presentation (Hsu et al. 1996). As an alternative strategy, which may be more practical for any future clinical use, the inventors will also consider using the peptide immunised directly along with an adjuvant such as poly(I:C), which has also been a strategy used clinically (Ott et al. 2017). As a back-up strategy, if revertant ID8 cells do not establish as tumour grafts (thus precluding the assessment of vaccines), the inventors will use an alternative strategy, by generating isogenic Brca-gene mutant/revertant 4T1 mouse mammary tumour cells, that can also be used in syngeneic animal experiments.

### Example 10—can Tumour Rejection be Improved by Vaccination or Immune Checkpoint Inhibition?

Finally, the inventors will also test treatment of established syngeneic tumours (with reversion mutations) with immune checkpoint inhibitors (anti-Ctla4 or anti-Pd1), with and without the reversion vaccine to further boost the host T cell response against the reverted tumour (FIG. 9C). If established tumours regress, the combination of vaccine and checkpoint inhibitor may also be considered as an effective treatment strategy for reverted tumours.

An important consideration in a vaccination strategy to prevent reversions is whether BRCA carriers might already have central immune tolerance to the out-of-frame sequence associated with reversions. In many reversion mutations there will be at least some sequence unique to the reversion mutation itself; however, most will also retain some of the out-of-frame sequence introduced by the original pathogenic mutation. This sequence would theoretically be shared with the primary tumour (in cases of somatic BRCA mutation) or with heterozygous normal cells in the body in carriers of BRCA germline mutations, and thus may have previously been exposed to the immune system during development of central tolerance. Whether this happens in practice is unknown. There is some evidence that the mutant BRCA-gene transcripts are degraded by nonsense-mediated decay and do not lead to production of stable protein; however some BRCA mutant cell lines such as Capan1 do express a truncated protein (Edwards et al. 2008). In order to unequivocally address this point, the inventors propose to assess whether healthy adult BRCA carriers have central tolerance to out-of-frame peptide sequence encoded by their germline mutant allele. This could be assessed by using the T-cell priming assay with T-cells from a BRCA carrier and peptides for the out-of-frame sequence. If T-cell clones that recognise the out-of-frame sequence have been negatively selected during development, BRCA carriers will not show a response in this assay whereas wild type donors will.

It is possible that stimulation of an immune response against reversions using a vaccine might result in development of auto-immunity in BRCA carriers in cases where the neoantigen sequence is shared between reversion and pathogenic mutations. To address this concern, the inventors will also use a recently developed mouse model carrying the mouse equivalent of the BRCA2:c.6174delT founder mutation (Brca2:c.5096delT). Vaccination of mice carrying this mutation (prior to development of any tumours) with the out-of-frame sequence from the pathogenic allele will allow observation of whether any ill effects occur.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

Afghahi, A., Timms, K. M., Vinayak, S., Jensen, K. C., Kurian, A. W., Carlson, R. W., Chang, P.-J., Schackmann, E., Hartman, A.-R., Ford, J. M., et al. (2017). Tumour BRCA1 Reversion Mutation Arising during Neoadjuvant Platinum-Based Chemotherapy in Triple-Negative Breast Cancer Is Associated with Therapy Resistance. Clin Cancer Res 23, 3365-3370.
Alsop, K., Fereday, S., Meldrum, C., deFazio, A., Emmanuel, C., George, J., Dobrovic, A., Birrer, M. J., Webb, P. M., Stewart, C., et al. (2012). BRCA mutation frequency and patterns of treatment response in BRCA mutation-positive women with ovarian cancer: a report from the Australian Ovarian Cancer Study Group. J Clin Oncol 30, 2654-2663.
Bailey, P., Chang, D. K., Nones, K., Johns, A. L., Patch, A.-M., Gingras, M.-C., Miller, D. K., Christ, A. N., Bruxner, T. J. C., Quinn, M. C., et al. (2016). Genomic analyses identify molecular subtypes of pancreatic cancer. Nature 531, 47-52.
Banda, K., Swisher, E. M., Wu, D., Pritchard, C. C., and Gadi, V. K. (2018). Somatic Reversion of Germline BRCA2 Mutation Confers Resistance to Poly(ADP-ribose) Polymerase Inhibitor Therapy. JCO Precision Oncology, 1-6.
Barber, L. J., Sandhu, S., Chen, L., Campbell, J., Kozarewa, I., Fenwick, K., Assiotis, I., Rodrigues, D. N., Reis Filho, J. S., Moreno, V., et al. (2013). Secondary mutations in BRCA2 associated with clinical resistance to a PARP inhibitor. J Pathol 229, 422-429.
Bhargava, R., Onyango, D. O., and Stark, J. M. (2016). Regulation of Single-Strand Annealing and its Role in Genome Maintenance. Trends in Genetics 32, 566-575.
Cancer Genome Atlas, N. (2012). Comprehensive molecular portraits of human breast tumours. Nature 490, 61-70.
Cancer Genome Atlas Research, N. (2011). Integrated genomic analyses of ovarian carcinoma. Nature 474, 609-615.
Carneiro, B. A., Collier, K. A., Nagy, R. J., Pamarthy, S., Sagar, V., Fairclough, S., Odegaard, J., Lanman, R. B., Costa, R., Taxter, T., et al. (2018). Acquired Resistance to Poly (ADP-ribose) Polymerase Inhibitor Olaparib in BRCA2-Associated Prostate Cancer Resulting From Biallelic BRCA2 Reversion Mutations Restores Both Germline and Somatic Loss-of-Function Mutations. JCO Precision Oncology, 1-8.
Cheng, H. H., Salipante, S. J., Nelson, P. S., Montgomery, B., and Pritchard, C. C. (2018). Polyclonal BRCA2 Reversion Mutations Detected in Circulating Tumour DNA After Platinum Chemotherapy in a Patient With Metastatic Prostate Cancer. JCO Precision Oncology, 1-5.
Christie, E. L., Fereday, S., Doig, K., Pattnaik, S., Dawson, S.-J., and Bowtell, D. D. L. (2017). Reversion of BRCA1/2 Germline Mutations Detected in Circulating Tumour DNA From Patients With High-Grade Serous Ovarian Cancer. J Clin Oncol 35, 1274-1280.
Cline, M. S., Liao, R. G., Parsons, M. T., Paten, B., Alquaddoomi, F., Antoniou, A., Baxter, S., Brody, L., Cook-Deegan, R., Coffin, A., et al. (2018). BRCA Challenge: BRCA Exchange as a global resource for variants in BRCA1 and BRCA2. PLoS Genet 14, e1007752-1007717.
Cruz, C., Castroviejo-Bermejo, M., Gutiérrez-Enríquez, S., Llop-Guevara, A., Ibrahim, Y. H., Gris-Oliver, A., Bonache, S., Morancho, B., Bruna, A., Rueda, O. M., et al. (2018). RAD51 foci as a functional biomarker of homologous recombination repair and PARP inhibitor resistance in germline BRCA-mutated breast cancer. Ann Oncol 29, 1203-1210.
Davies, H., Glodzik, D., Morganella, S., Yates, L. R., Staaf, J., Zou, X., Ramakrishna, M., Martin, S., Boyault, S., Sieuwerts, A. M., et al. (2017). HRDetect is a predictor of BRCA1 and BRCA2 deficiency based on mutational signatures. Nat Med 23, 517-525.
Dréan, A., Williamson, C. T., Brough, R., Brandsma, I., Menon, M., Konde, A., Garcia-Murillas, I., Pemberton, H. N., Frankum, J., Rafiq, R., et al. (2017). Modeling Therapy Resistance in BRCA1/2-Mutant Cancers. Mol Cancer Ther 16, 2022-2034.
Edwards, S. L., Brough, R., Lord, C. J., Natrajan, R., Vatcheva, R., Levine, D. A., Boyd, J., Reis-Filho, J. S., and Ashworth, A. (2008). Resistance to therapy caused by intragenic deletion in BRCA2. Nature 451, 1111-1115.
Esashi, F., Galkin, V. E., Yu, X., Egelman, E. H., and West, S. C. (2007). Stabilization of RAD51 nucleoprotein filaments by the C-terminal region of BRCA2. Nat Struct Mol Biol 14, 468-474.
Futreal, P. A., Liu, Q., Shattuck-Eidens, D., Cochran, C., Harshman, K., Tavtigian, S., Bennett, L. M., Haugen-Strano, A., Swensen, J., Miki, Y., et al. (1994). BRCA1 mutations in primary breast and ovarian carcinomas. Science 266, 120-122.
Goodall, J., Mateo, J., Yuan, W., Mossop, H., Porta, N., Miranda, S., Perez-Lopez, R., Dolling, D., Robinson, D. R., Sandhu, S., et al. (2017). Circulating Cell-Free DNA to Guide Prostate Cancer Treatment with PARP Inhibition. Cancer Discov 7, 1006-1017.
Gornstein, E. L., Sandefur, S., Chung, J. H., Gay, L. M., Holmes, O., Erlich, R. L., Soman, S., Martin, L. K., Rose, A. V., Stephens, P. J., et al. (2018). BRCA2 Reversion Mutation Associated With Acquired Resistance to Olaparib in Estrogen Receptor-positive Breast Cancer Detected by Genomic Profiling of Tissue and Liquid Biopsy. Clin Breast Cancer 18, 184-188.
Gourraud, P.-A., Khankhanian, P., Cereb, N., Yang, S. Y., Feolo, M., Maiers, M., Rioux, J. D., Hauser, S., and Oksenberg, J. (2014). HLA diversity in the 1000 genomes dataset. PLoS ONE 9, e97282.
Grasso, C. S., Grasso, C. S., Wu, Y.-M., Wu, Y.-M., Robinson, D. R., Robinson, D. R., Cao, X., Cao, X., Dhanasekaran, S. M., Dhanasekaran, S. M., et al. (2012). The mutational landscape of lethal castration-resistant prostate cancer. Nature 487, 239-243.
Holter, S., Borgida, A., Dodd, A., Grant, R., Semotiuk, K., Hedley, D., Dhani, N., Narod, S., Akbari, M., Moore, M., et al. (2015). Germline BRCAMutations in a Large Clinic-Based Cohort of Patients With Pancreatic Adenocarcinoma. Journal of Clinical Oncology 33, 3124-3129.
Ikeda, H., Matsushita, M., Waisfisz, Q., Kinoshita, A., Oostra, A. B., Nieuwint, A. W. M., De Winter, J. P., Hoatlin, M. E., Kawai, Y., Sasaki, M. S., et al. (2003). Genetic reversion in an acute myelogenous leukemia cell line from a Fanconi anemia patient with biallelic mutations in BRCA2. Cancer Res 63, 2688-2694.
Jurtz, V., Paul, S., Andreatta, M., Marcatili, P., Peters, B., and Nielsen, M. (2017). NetMHCpan-4.0: Improved Peptide-MHC Class I Interaction Predictions Integrating Eluted Ligand and Peptide Binding Affinity Data. J Immunol 199, 3360-3368.
Khalique, S., Pettitt, S. J., Kelly, G., Tunariu, N., Natrajan, R., Banerjee, S., and Lord, C. J. (2019). Longitudinal analysis of a secondary BRCA2 mutation using digital droplet PCR. J Pathol Clin Res.
Kondrashova, O., Nguyen, M., Shield-Artin, K., Tinker, A. V., Teng, N. N. H., Harrell, M. I., Kuiper, M. J., Ho, G.-Y., Barker, H., Jasin, M., et al. (2017). Secondary Somatic Mutations Restoring and Associated with Acquired Resistance to the PARP Inhibitor Rucaparib in High-Grade Ovarian Carcinoma. Cancer Discov 7, 984-998.
Lancaster, J. M., Wooster, R., Mangion, J., Phelan, C. M., Cochran, C., Gumbs, C., Seal, S., Barfoot, R., Collins, N., Bignell, G., et al. (1996). BRCA2 mutations in primary breast and ovarian cancers. Nat Genet 13, 238-240.
Landrum, M. J., Lee, J. M., Benson, M., Brown, G. R., Chao, C., Chitipiralla, S., Gu, B., Hart, J., Hoffman, D., Jang, W., et al. (2017). ClinVar: improving access to variant interpretations and supporting evidence. Nucleic acids research 46, D1062-D1067.
Lin, K. K., Harrell, M. I., Oza, A. M., Oaknin, A., Ray-Coquard, I., Tinker, A. V., Helman, E., Radke, M. R., Say, C., Vo, L.-T., et al. (2019). Reversion Mutations in Circulating Tumour DNA Predict Primary and Acquired Resistance to the PARP Inhibitor Rucaparib in High-Grade Ovarian Carcinoma. Cancer Discov 9, 210-219.
Livingstone, C. D., and Barton, G. J. (1993). Protein sequence alignments: a strategy for the hierarchical analysis of residue conservation. Comput Appl Biosci 9, 745-756.
Lord, C. J., and Ashworth, A. (2016). BRCAness revisited. Nat Rev Cancer 16, 110-120.
Lord, C. J., and Ashworth, A. (2017). PARP inhibitors: Synthetic lethality in the clinic. Science 355, 1152-1158.
Marty, R., Kaabinejadian, S., Rossell, D., Slifker, M. J., van de Haar, J., Engin, H. B., de Prisco, N., Ideker, T., Hildebrand, W. H., Font-Burgada, J., et al. (2017). MHC-I Genotype Restricts the Oncogenic Mutational Landscape. Cell 171, 1272-1283.e1215.
Mayor, P., Gay, L. M., Lele, S., and Elvin, J. A. (2017). reversion mutation acquired after treatment identified by liquid biopsy. Gynecol Oncol Rep 21, 57-60.
Meijer, T. G., Verkaik, N. S., van Deurzen, C. H. M., Dubbink, H.-J., den Toom, T. D., Sleddens, H. F. B. M., De Hoop, E. O., Dinjens, W. N. M., Kanaar, R., van Gent, D. C., et al. (2019). Direct Ex Vivo Observation of Homologous Recombination Defect Reversal After DNA-Damaging Chemotherapy in Patients With Metastatic Breast Cancer. JCO Precision Oncology, 1-12.
Norquist, B., Wurz, K. A., Pennil, C. C., Garcia, R., Gross, J., Sakai, W., Karlan, B. Y., Taniguchi, T., and Swisher, E. M. (2011). Secondary somatic mutations restoring BRCA1/2 predict chemotherapy resistance in hereditary ovarian carcinomas. J Clin Oncol 29, 3008-3015.
Patch, A.-M., Christie, E. L., Etemadmoghadam, D., Garsed, D. W., George, J., Fereday, S., Nones, K., Cowin, P., Alsop, K., Bailey, P. J., et al. (2015). Whole-genome characterization of chemoresistant ovarian cancer. Nature 521, 489-494.
Patel, J. N., Braicu, I., Timms, K. M., Solimeno, C., Tshiaba, P., Reid, J., Lanchbury, J. S., Darb-Esfahani, S., Ganapathi, M. K., Sehouli, J., et al. (2018). Characterisation of homologous recombination deficiency in paired primary and recurrent high-grade serous ovarian cancer. Br J Cancer 119, 1060-1066.
Pishvaian, M. J., Biankin, A. V., Bailey, P., Chang, D. K., Laheru, D., Wolfgang, C. L., and Brody, J. R. (2017). BRCA2 secondary mutation-mediated resistance to platinum and PARP inhibitor-based therapy in pancreatic cancer. Br J Cancer 116, 1021-1026.
Powell, S., Forslund, K., Szklarczyk, D., Trachana, K., Roth, A., Huerta-Cepas, J., Gabaldón, T., Rattei, T., Creevey, C., Kuhn, M., et al. (2014). eggNOG v4.0: nested orthology inference across 3686 organisms. Nucleic acids research 42, D231-239.
Punta, M., Jennings, V., Melcher, A., and Lise, S. (2019). The immunogenic potential of recurrent cancer drug resistance mutations: an in silico study. biorxiv, 1-22.
Quigley, D., Alumkal, J. J., Wyatt, A. W., Kothari, V., Foye, A., Lloyd, P., Aggarwal, R., Kim, W., Lu, E., Schwartzman, J., et al. (2017). Analysis of Circulating Cell-Free DNA Identifies Multiclonal Heterogeneity of Reversion Mutations Associated with Resistance to PARP Inhibitors. Cancer Discov 7, 999-1005.
Sakai, W., Swisher, E. M., Jacquemont, C., Chandramohan, K. V., Couch, F. J., Langdon, S. P., Wurz, K., Higgins, J., Villegas, E., and Taniguchi, T. (2009). Functional restoration of BRCA2 protein by secondary BRCA2 mutations in BRCA2-mutated ovarian carcinoma. Cancer Res 69, 6381-6386.
Sakai, W., Swisher, E. M., Karlan, B. Y., Agarwal, M. K., Higgins, J., Friedman, C., Villegas, E., Jacquemont, C., Farrugia, D. J., Couch, F. J., et al. (2008). Secondary mutations as a mechanism of cisplatin resistance in BRCA2-mutated cancers. Nature 451, 1116-1120.
Shroff, R. T., Hendifar, A., McWilliams, R. R., Geva, R., Epelbaum, R., Rolfe, L., Goble, S., Lin, K. K., Biankin, A. V., Giordano, H., et al. (2018). Rucaparib Monotherapy in Patients With Pancreatic Cancer and a Known Deleterious Mutation. JCO Precis Oncol 2018.
Sinha, S., Villarreal, D., Shim, E. Y., and Lee, S. E. (2016). Risky business: Microhomology-mediated end joining. Mutat Res 788, 17-24.
Spurdle, A. B., Healey, S., Devereau, A., Hogervorst, F. B. L., Monteiro, A. N. A., Nathanson, K. L., Radice, P., Stoppa-Lyonnet, D., Tavtigian, S., Wappenschmidt, B., et al. (2011). ENIGMA-Evidence-based network for the interpretation of germline mutant alleles: An international initiative to evaluate risk and clinical significance associated with sequence variation in BRCA1 and BRCA2 genes. Hum Mutat 33, 2-7.
Staaf, J., Glodzik, D., Bosch, A., Vallon-Christersson, J., Reuterswärd, C., Häkkinen, J., Degasperi, A., Amarante, T. D., Saal, L. H., Hegardt, C., et al. (2019). Whole-genome sequencing of triple-negative breast cancers in a population-based clinical study. Nat Med 25, 1526-1533.
Swisher, E. M., Sakai, W., Karlan, B. Y., Wurz, K., Urban, N., and Taniguchi, T. (2008). Secondary BRCA1 mutations in BRCA1-mutated ovarian carcinomas with platinum resistance. Cancer Res 68, 2581-2586.
Ter Brugge, P., Kristel, P., van der Burg, E., Boon, U., de Maaker, M., Lips, E., Mulder, L., de Ruiter, J., Moutinho, C., Gevensleben, H., et al. (2016). Mechanisms of Therapy Resistance in Patient-Derived Xenograft Models of BRCA1-Deficient Breast Cancer. J Natl Cancer Inst 108.
Tutt, A. (2018). Inhibited, trapped or adducted: the optimal selective synthetic lethal mix for BRCAness. Annals of oncology: official journal of the European Society for Medical Oncology/ESMO 29, 18-21.
Tutt, A., Tovey, H., Cheang, M. C. U., Kernaghan, S., Kilburn, L., Gazinska, P., Owen, J., Abraham, J., Barrett, S., Barrett-Lee, P., et al. (2018). Carboplatin in BRCA1/2-mutated and triple-negative breast cancer BRCAness subgroups: the TNT Trial. Nat Med 24, 628-637.
Waddell, N., Pajic, M., Patch, A.-M., Chang, D. K., Kassahn, K. S., Bailey, P., Johns, A. L., Miller, D., Nones, K., Quek, K., et al. (2015). Whole genomes redefine the mutational landscape of pancreatic cancer. Nature 518, 495-501.
Wang, Y., Bernhardy, A. J., Cruz, C., Krais, J. J., Nacson, J., Nicolas, E., Peri, S., van der Gulden, H., van der Heijden, I., O'Brien, S. W., et al. (2016). The BRCA1-Δ11q Alternative Splice Isoform Bypasses Germline Mutations and Promotes Therapeutic Resistance to PARP Inhibition and Cisplatin. Cancer Res 76, 2778-2790.
Weigelt, B., Comino-Méndez, I., de Bruijn, I., Tian, L., Meisel, J. L., García-Murillas, I., Fribbens, C., Cutts, R., Martelotto, L. G., Ng, C. K. Y., et al. (2017). Diverse and Reversion Mutations in Circulating Cell-Free DNA of Therapy-Resistant Breast or Ovarian Cancer. Clin Cancer Res 23, 6708-6720.
Yates, A., Akanni, W., Amode, M. R., Barrell, D., Billis, K., Carvalho-Silva, D., Cummins, C., Clapham, P., Fitzgerald, S., Gil, L., et al. (2016). Ensembl 2016. Nucleic acids research 44, D710-716.
Yun, M. H., and Hiom, K. (2009). CtIP-BRCA1 modulates the choice of DNA double-strand-break repair pathway throughout the cell cycle. Nature 459, 460-463.
Alsop, Kathryn, Sian Fereday, Cliff Meldrum, Anna deFazio, Catherine Emmanuel, Joshy George, Alexander Dobrovic, et al. 2012. “BRCA Mutation Frequency and Patterns of Treatment Response in BRCA Mutation-Positive Women with Ovarian Cancer: A Report from the Australian Ovarian Cancer Study Group.” Journal of Clinical Oncology 30 (21): 2654-63.
Christie, Elizabeth L., Sian Fereday, Ken Doig, Swetansu Pattnaik, Sarah-Jane Dawson, and David D. L. Bowtell. 2017. “Reversion of BRCA1/2 Germline Mutations Detected in Circulating Tumour DNA From Patients With High-Grade Serous Ovarian Cancer.” Journal of Clinical Oncology 35 (12): 1274-80.
Dréan, Amy, Chris T. Williamson, Rachel Brough, Inger Brandsma, Malini Menon, Asha Konde, Isaac Garcia-Murillas, et al. 2017. “Modeling Therapy Resistance in BRCA1/2-Mutant Cancers.” Molecular Cancer Therapeutics 16 (9): 2022-34.
Edwards, Stacey L., Rachel Brough, Christopher J. Lord, Rachael Natrajan, Radost Vatcheva, Douglas A. Levine, Jeff Boyd, Jorge S. Reis-Filho, and Alan Ashworth. 2008. “Resistance to Therapy Caused by Intragenic Deletion in BRCA2.” Nature 451 (7182): 1111-15.
Hsu, Frank J., Claudia Benike, Francesco Fagnoni, Tina Marie Liles, Debra Czerwinski, Behnaz Taidi, Edgar G. Engleman, and Ronald Levy. 1996. “Vaccination of Patients with B-cell Lymphoma Using Autologous Antigen-pulsed Dendritic Cells.” Nature Medicine 2 (1) 52-58.
Jennings, Victoria A., Gina B. Scott, Ailsa M. S. Rose, Karen J. Scott, Gemma Migneco, Brian Keller, Katrina Reilly, et al. 2019. “Potentiating Oncolytic Virus-Induced Immune-Mediated Tumour Cell Killing Using Histone Deacetylase Inhibition.” Molecular Therapy 27 (6): 1139-52.
Jurtz, Vanessa, Sinu Paul, Massimo Andreatta, Paolo Marcatili, Bjoern Peters, and Morten Nielsen. 2017. “NetMHCpan 4.0: Improved Peptide-MHC Class I Interaction Predictions Integrating Eluted Ligand and Peptide Binding Affinity Data.” BioRXiv. https://doi.org/10.1101/149518.
Lin, Kevin K., Maria I. Harrell, Amit M. Oza, Ana Oaknin, Isabelle Ray-Coquard, Anna V. Tinker, Elena Helman, et al. 2019. “Reversion Mutations in Circulating Tumour DNA Predict Primary and Acquired Resistance to the PARP Inhibitor Rucaparib in High-Grade Ovarian Carcinoma.” Cancer Discovery 9 (2): 210-19.
Lord, Christopher J., and Alan Ashworth. 2017. “PARP Inhibitors: Synthetic Lethality in the Clinic.” Science 355 (6330): 1152-58.
Ott, Patrick A., Zhuting Hu, Derin B. Keskin, Sachet A. Shukla, Jing Sun, David J. Bozym, Wandi Zhang, et al. 2017. “An Immunogenic Personal Neoantigen Vaccine for Patients with Melanoma.” Nature 547 (7662): 217-21.
Pettitt, Stephen J., and Christopher J. Lord. 2019. “Dissecting PARP Inhibitor Resistance with Functional Genomics.” Current Opinion in Genetics & Development 54:55-63.
Prestwich, Robin J., Fiona Errington, Elizabeth J. Ilett, Ruth S. M. Morgan, Karen J. Scott, Timothy Kottke, Jill Thompson, et al. 2008. “Tumour Infection by Oncolytic Reovirus Primes Adaptive Antitumour Immunity.” Clinical Cancer Research 14 (22): 7358-66.
Punta, Marco, Victoria Jennings, Alan Melcher, and Stefano Lise. 2019. “The Immunogenic Potential of Recurrent Cancer Drug Resistance Mutations: An in Silico Study.” BioRXiv https://doi.org/10.1101/845784
Sakai, Wataru, Elizabeth M. Swisher, Beth Y. Karlan, Mukesh K. Agarwal, Jake Higgins, Cynthia Friedman, Emily Villegas, et al. 2008. “Secondary Mutations as a Mechanism of Cisplatin Resistance in BRCA2-Mutated Cancers.” Nature 451 (7182): 1116-20.
Tutt, Andrew, Holly Tovey, Maggie Chon U. Cheang, Sarah Kernaghan, Lucy Kilburn, Patrycja Gazinska, Julie Owen, et al. 2018. “Carboplatin in BRCA1/2-Mutated and Triple-Negative Breast Cancer BRCAness Subgroups: The TNT Trial.” Nature Medicine 24 (5): 628-37.
Walton, Josephine B., Malcolm Farquharson, Susan Mason, Jennifer Port, Bjorn Kruspig, Suzanne Dowson, David Stevenson, et al. 2017. “CRISPR/Cas9-Derived Models of Ovarian High Grade Serous Carcinoma Targeting Brca1, Pten and Nfl, and Correlation with Platinum Sensitivity.” Scientific Reports 7 (1): 16827.
Walton, Josephine, Julianna Blagih, Darren Ennis, Elaine Leung, Suzanne Dowson, Malcolm Farquharson, Laura A. Tookman, et al. 2016. “CRISPR/Cas9-Mediated Trp53 and Brca2 Knockout to Generate Improved Murine Models of Ovarian High-Grade Serous Carcinoma.” Cancer Research 76 (20): 6118-29.

1. An anti-cancer vaccine comprising:
(i) at least one peptide comprising the amino acid sequence of a neoantigen encoded by a mutant Homologous Recombination (HR) DNA repair gene selected from the group: BRCA1, BRCA2, PALB2, CDK12, RAD51B, RAD51C and RAD51D, wherein said mutant gene comprises a reversion mutation; and/or
(ii) at least one polynucleotide encoding the at least one peptide of (i).

2. The anti-cancer vaccine of claim 1, wherein said neoantigen comprises an amino acid sequence selected from the sequences set forth in SEQ ID Nos: 1-1218, 1223-1227 and/or 1231-1247.

3. The anti-cancer vaccine of claim 1 or claim 2, wherein said neoantigen comprises an amino acid sequence selected from the neopeptide sequences set forth in Table 5 and Table 6.

4. The anti-cancer vaccine of any one of claims 1-3, wherein said neoantigen comprises an amino acid sequence selected from the sequences set forth as SEQ ID Nos: 1-89.

5. The anti-cancer vaccine of claim 4, wherein said neoantigen comprises the amino acid sequence: SRYQMLHYKTQ (SEQ ID NO: 1212) or RENLSRYQMLHYKTQ (SEQ ID NO: 1247).

6. The anti-cancer vaccine of any one of the preceding claims, wherein the vaccine comprises a DNA or RNA sequence encoding said neoantigen.

7. The anti-cancer vaccine of claim 6, wherein the DNA or RNA sequence is provided in the form of a viral vector.

8. The anti-cancer vaccine of claim 7, wherein the viral vector is an oncolytic virus.

9. The anti-cancer vaccine of any one of the preceding claims, wherein said vaccine is in the form of a plurality of dendritic cells (DCs) that have been pulsed with said at least one peptide comprising the neoantigen and which are capable of presenting said neoantigen to one or more T cells when administered to a subject.

10. The anti-cancer vaccine of any one of the preceding claims, wherein said neoantigen is an MHC class I restricted peptide.

11. The anti-cancer vaccine of claim 10, wherein said neoantigen is predicted to be presented by at least 10%, 25%, 50% or at least 75% of the HLA-A, HLA-B and/or HLA-C allotypes of the 1000 Genomes dataset.

12. The anti-cancer vaccine of any one of the preceding claims, wherein said neoantigen is predicted to be presented by MHC class I with a best rank (BR) score of 0.5 or less using the NetMHCpan-4.0 neural network predictor.

13. The anti-cancer vaccine of any one of the preceding claims, wherein the vaccine comprises 2, 3, 4, 5, 6, 7, 8, 9, 10 or more different neoantigens and/or polynucleotide encoding the different neoantigens.

14. The anti-cancer vaccine of any one of the preceding claims further comprising at least one adjuvant.

15. The anti-cancer vaccine of claim 14, wherein said at least one adjuvant is selected from: a toll-like receptor (TLR) agonist.

16. An engineered T cell that recognises a neoantigen encoded by a mutant HR DNA repair gene selected from the group: BRCA1, BRCA2, PALB2, CDK12, RAD51B, RAD51C and RAD51D, wherein the mutant gene comprises a reversion mutation and wherein the T cell is selected from: a chimeric antigen receptor T cell (CAR-T), an engineered T cell receptor (TCR) T cell or a neoantigen-reactive T cell (NAR-T).

17. The engineered T cell of claim 16, wherein the neoantigen is as defined in any one of claims 1 to 15.

18. The anti-cancer vaccine of any one of claims 1 to 15 or the engineered T cell of claim 16 or claim 17 for use in medicine.

19. The anti-cancer vaccine of any one of claims 1 to 15 or the engineered T cell of claim 16 or claim 17 for use in a method of treatment of a proliferative disorder in a mammalian subject.

20. The anti-cancer vaccine or engineered T cell for use of claim 19, wherein the proliferative disorder is a cancer that exhibits a homologous recombination (HR) defect.

21. The anti-cancer vaccine or engineered T cell for use of claim 19 or claim 20, wherein the proliferative disorder is selected from: high grade serous ovarian cancer (HGSOC), triple-negative breast cancer (TNBC), castrate resistant metastatic prostate cancer and pancreatic cancer.

22. The anti-cancer vaccine or engineered T cell for use of any one of claims 19 to 21, wherein the method of treatment comprises the inhibition or prevention of development of, or reduction or reversal of tumour resistance to PARP inhibitor therapy and/or platinum therapy.

23. The anti-cancer vaccine or engineered T cell for use of any one of claims 19 to 22, wherein the subject has a mutation in the BRCA1 gene, BRCA2 gene, PALB2 gene, CDK12 gene, RAD51B gene, RAD51C gene and/or RAD51D gene.

24. The anti-cancer vaccine or engineered T cell for use of claim 23, wherein the mutation in the BRCA1 gene, BRCA2 gene, PALB2 gene, CDK12 gene, RAD51B gene, RAD51C gene and/or RAD51D gene is a somatic mutation.

25. The anti-cancer vaccine or engineered T cell for use of claim 23 or claim 24, wherein the BRCA1 mutation, the BRCA2 mutation, the PALB2 mutation, the CDK12 mutation, RAD51B mutation, RAD51C mutation and/or the RAD51D mutation is a reversion mutation, optionally wherein the reversion mutation is as set forth in Table 3, Table 5 or Table 6.

26. The anti-cancer vaccine or engineered T cell for use of any one of claims 23 to 25, wherein said method of treatment comprises a step of determining whether said BRCA1 mutation, said BRCA2 mutation, said PALB2 mutation, said CDK12 mutation, RAD51B mutation, RAD51C mutation and/or said RAD51D mutation is present in a tumour of the subject.

27. The anti-cancer vaccine or engineered T cell for use of claim 26, wherein determining whether said mutation is present in a tumour of the subject comprises sequencing ctDNA from a sample obtained from the subject.

28. The anti-cancer vaccine or engineered T cell for use of claim 26 or claim 27, wherein the tumour is determined have a BRCA1 gene, BRCA2 gene, PALB2 gene, CDK12 gene, RAD51B gene, RAD51C gene or RAD51D gene with a reversion mutation in said gene that encodes a gene product comprising a neoantigen amino acid sequence, optionally wherein the neoantigen amino acid sequence is as defined in any one of claims 2 to 5.

29. The anti-cancer vaccine or engineered T cell for use of any one of claims 19 to 26, wherein the subject is undergoing or is a candidate to undergo therapy with a PARP inhibitor and/or a platinum-based chemotherapeutic.

30. The anti-cancer vaccine or engineered T cell for use of any one of claims 19 to 29, wherein the method of treatment is a combination therapy that further comprises treatment with an immune checkpoint inhibitor.

31. The anti-cancer vaccine or engineered T cell for use of any one of claims 19 to 30, wherein the method of treatment is a combination therapy that further comprises treatment with radiotherapy and/or chemotherapy.

32. The anti-cancer vaccine or engineered T cell for use of any one of claims 19 to 31, wherein the method of treatment is a combination therapy that comprises treatment with both said anti-cancer vaccine and said engineered T cell.

33. The anti-cancer vaccine or engineered T cell for use of any one of claims 19 to 31, wherein the method of treatment further comprises a step of HLA typing the subject and matching the neoantigen to the HLA allotype of the subject.

34. The anti-cancer vaccine or engineered T cell for use of claim 33, wherein matching the neoantigen to the HLA allotype of the subject comprises a step of predicting MHC class I presentation of the neoantigen sequence by the subject, wherein said predicting employs a computational tool.

35. The anti-cancer vaccine or engineered T cell for use of claim 34, wherein said computational tool comprises NetMHCpan-4.

36. A method for treatment of a proliferative disorder in a mammalian subject in need thereof, comprising administering a therapeutically effective amount of an anti-cancer vaccine as defined in any one of claims 1 to 15 or an engineered T cell as defined in claim 16 or claim 17 to the subject.
